(19) United States
(12) Patent Application Publication
(10) Pub. No.: US 2024/0409635 A1
(43) Pub. Date: Dec. 12, 2024

(54) MULTISPECIFIC ANTIBODIES AND USES THEREOF

(71) Applicant: Vibrant Pharma Limited, Guangzhou (CN)

(72) Inventors: Changhua Zhou, Guangzhou (CN); Hong Zhang, Guangzhou (CN); Luquan Wang, Guangzhou (CN)

(21) Appl. No.: 18/698,900

(22) PCT Filed: May 15, 2023
(86) PCT No.: PCT/CN2023/094287

§ 371 Date: Apr. 5, 2024

(30) Foreign Application Priority Data
May 13, 2022 (WO) PCT/CN2022/092867

(51) Int. Cl. C07K 16/28; A61K 39/00; A61P 35/00; C07K 16/30; C07K 16/32

(52) U.S. Cl. CPC C07K 16/2809; A61P 35/00; C07K 16/2863; C07K 16/3007; C07K 16/32; A61K 2039/505; C07K 2317/22; C07K 2317/24; C07K 2317/31; C07K 2317/53; C07K 2317/55; C07K 2317/569; C07K 2317/73; C07K 2317/92; C07K 2317/94

(57) ABSTRACT

This disclosure relates to multispecific antibodies (e.g., bispecific antibodies or trispecific antibodies) or antigen-binding fragments thereof. In one aspect, the multispecific antibodies or antigen-binding fragments thereof can bind to a T cell antigen (e.g., CD3) and/or one or two tumor-associated antigens (e.g., CEA-CAM6, EGFR, HER2), or a combination thereof.

### TECHNICAL FIELD

This disclosure relates to multispecific antibodies or antigen-binding fragments thereof.

### BACKGROUND

Naturally occurring antibodies typically only target one antigen. A multispecific antibody can be manufactured in different structural formats, so that they can simultaneously bind to two or more different epitopes. These epitopes can be in the same antigen or in different antigen. This opens up a wide range of applications, including redirecting T cells to tumor cells, blocking two different signaling pathways simultaneously, dual targeting of different disease mediators, and delivering payloads to targeted sites.

Multispecific antibodies have various applications. However, in some cases, a multispecific antibody may not have the desired efficacy and it can be difficult to express and purify. There is a need to continue to develop various therapeutics based on multispecific antibodies.

### SUMMARY

This disclosure relates to multispecific antibodies or antigen-binding fragments thereof, wherein the multispecific antibodies or antigen-binding fragments thereof specifically bind to a T cell antigen (e.g., CD3) and/or one or more tumor-associated antigens, or a combination thereof.

In one aspect, the disclosure is related to an antigen-binding protein, comprising
(a) a Fe;
(b) a Fab fragment (Fab) that specifically binds to a T cell antigen; and
(c) a first single-domain antibody variable domain (VHH) that specifically binds to a first tumor-associated antigen;
(d) a second single-domain antibody variable domain (VHH) that specifically binds to a second tumor-associated antigen.

In some embodiments, the Fab, the first VHH, and the second VHH are linked to the Fc.

In some embodiments, the Fab comprises or consists of a light chain variable domain (VL), a light chain constant domain (CL), a heavy chain variable domain (VH), and a heavy chain first constant domain (CH1).

In some embodiments, the Fab can activate T cells upon binding to the T cell antigen.

In some embodiments, the T cell antigen is cluster of differentiation 3 (CD3).

In some embodiments, the first tumor-associated antigen and the second tumor-associated antigen are independently selected from the group consisting of cluster of differentiate 20 (CD20), carcinoembryonic antigen (CEA), prostate-specific antigen (PSA), prostate stem cell antigen (PSCA), programmed death-ligand 1 (PD-L1), human epidermal growth factor receptor 2 (HER2), human epidermal growth factor receptor 3 (Her3), human epidermal growth factor receptor (Hern), β-Catenin, cluster of differentiate 19 (CD19), epidermal growth factor receptor (EGFR), tyrosine-protein kinase Met (c-Met), epithelial cell adhesion molecule (EPCAM), prostate-specific membrane antigen (PSMA), cluster of differentiate 40 (CD40), Mucin 1, Cell Surface Associated (MUC1), insulin-like growth factor 1 receptor (IGF1R), and carcinoembryonic antigen cell adhesion molecule 6 (CEA-CAM6).

In some embodiments, the Fc is human IgG4 Fe.

In some embodiments, the CH1 domain of the Fab is linked to a CH2 domain in the Fc, optionally via a hinge region.

In some embodiments, the hinge region is a human IgG4 hinge region optionally with S228P mutation according to EU numbering.

In some embodiments, the first VHH is linked to a CH2 domain in the Fc, optionally via a hinge region.

In some embodiments, the first VHH is linked to a CH3 domain in the Fc, optionally via a linker sequence.

In some embodiments, the second VHH is linked to a CH3 domain in the Fc, optionally via a linker sequence.

In some embodiments, the second VHH is linked to the Fab.

In some embodiments, the second VHH is linked to the C-terminus of a CL domain in the Fab.

In some embodiments, the second VHH is linked to the N-terminus of a VH domain in the Fab.

In some embodiments, the second VHH is linked to the N-terminus of a VL domain in the Fab.

In one aspect, the disclosure is related to a protein complex, comprising:
(a) a first polypeptide comprising in the direction of N-terminus to C-terminus: a VH, a CH1 domain, optionally a first hinge region, a first CH2 domain, and a first CH3 domain;
(b) a second polypeptide comprising in the direction of N-terminus to C-terminus: a first VHH, optionally a second hinge region, a second CH2 domain, and a second CH3 domain;
(c) a third polypeptide comprising in the direction of N-terminus to C-terminus: a VL, and a CL domain.

In some embodiments, the VH and the VL associate with each other, forming an antigen binding site of a Fab that specifically binds to a T cell antigen. In some embodiments, the first VHH specifically binds to a first tumor-associated antigen.

In some embodiments, the third polypeptide comprises in the direction of N-terminus to C-terminus: a VL, a CL domain, and a second VHH. In some embodiments, the second VHH specifically binds to a second tumor-associated antigen.

In some embodiments, the third polypeptide comprises in the direction of N-terminus to C-terminus: a second VHH, a VL and a CL domain. In some embodiments, the second VHH specifically binds to a second tumor-associated antigen.

In some embodiments, the first polypeptide comprises in the direction of N-terminus to C-terminus: a second VHH, a VH, a CH1 domain, optionally a first hinge region, a first CH2 domain, and a first CH3 domain. In some embodiments, the second VHH specifically binds to a second tumor-associated antigen.

In some embodiments, the second polypeptide comprises in the direction of N-terminus to C-terminus: a first VHH, a second VHH, optionally a second hinge region, a second CH2 domain, and a second CH3 domain. In some embodiments, the second VHH specifically binds to a second tumor-associated antigen.

In some embodiments, the second polypeptide comprises in the direction of N-terminus to C-terminus: a first VHH, optionally a second hinge region, a second CH2 domain, a second CH3 domain, and a second VHH. In some embodiments, the second VHH specifically binds to a second tumor-associated antigen.

In some embodiments, the first polypeptide comprises in the direction of N-terminus to C-terminus: a first VHH, a second VHH, optionally a second hinge region, a second CH2 domain, a second CH3 domain, and a second VHH. In some embodiments, the second VHH specifically binds to a second tumor-associated antigen.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

In some embodiments, the T cell antigen is CD3, and the first tumor-associated antigen is selected from the group consisting of: CEA, CEA-CAM6, HER2 and EGFR.

In some embodiments, the T cell antigen is CD3, and the second tumor-associated antigen is selected from the group consisting of: CEA, CEA-CAM6, HER2 and EGFR In one aspect, the disclosure is related to a protein complex, comprising:
(a) a first polypeptide comprising in the direction of N-terminus to C-terminus: a first VHH, optionally a first hinge region, a first CH2 domain, and a first CH3 domain;
(b) a second polypeptide comprising in the direction of N-terminus to C-terminus: a VH, a CH1 domain, optionally a second hinge region, a second CH2 domain, and a second CH3 domain;
(c) a third polypeptide comprising in the direction of N-terminus to C-terminus: a VL, and a CL domain.

In some embodiments, the VH and the VL associate with each other, forming an antigen binding site of a Fab that specifically binds to a T cell antigen. In some embodiments, the first VHH specifically binds to a first tumor-associated antigen.

In some embodiments, the third polypeptide comprises in the direction of N-terminus to C-terminus: a VL, a CL domain, and a second VHH. In some embodiments, the second VHH specifically binds to a second tumor-associated antigen.

In some embodiments, the third polypeptide comprises in the direction of N-terminus to C-terminus: a second VHH, a VL and a CL domain. In some embodiments, the second VHH specifically binds to a second tumor-associated antigen.

In some embodiments, the second polypeptide comprises in the direction of N-terminus to C-terminus: a second VHH, a VH, a CH1 domain, optionally a first hinge region, a first CH2 domain, and a first CH3 domain. In some embodiments, the second VHH specifically binds to a second tumor-associated antigen.

In some embodiments, the first polypeptide comprises in the direction of N-terminus to C-terminus: a first VHH, a second VHH, optionally a second hinge region, a second CH2 domain, and a second CH3 domain. In some embodiments, the second VHH specifically binds to a second tumor-associated antigen.

In some embodiments, the second polypeptide comprises in the direction of N-terminus to C-terminus: a VH, a CH1 domain, optionally a first hinge region, a first CH2 domain, a first CH3 domain, and a second VHH. In some embodiments, the second VHH specifically binds to a second tumor-associated antigen.

In some embodiments, the first polypeptide comprises in the direction of N-terminus to C-terminus: a first VHH, optionally a second hinge region, a second CH2 domain, a second CH3 domain, and a second VHH. In some embodiments, the second VHH specifically binds to a second tumor-associated antigen.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

In some embodiments, the T cell antigen is CD3, and the first tumor-associated antigen is selected from the group consisting of: CEA, CEA-CAM6, HER2 and EGFR.

In some embodiments, the T cell antigen is CD3, and the second tumor-associated antigen is selected from the group consisting of: CEA, CEA-CAM6, HER2 and EGFR.

In one aspect, the disclosure is related to a protein complex, comprising:
(a) a first polypeptide comprising in the direction of N-terminus to C-terminus: a VH, a CH1 domain, optionally a first hinge region, a first CH2 domain, a first CH3 domain, and a first VHH;
(b) a second polypeptide comprising in the direction of N-terminus to C-terminus: optionally a second hinge region, a second CH2 domain, a second CH3 domain, and a second VHH; and
(c) a third polypeptide comprising in the direction of N-terminus to C-terminus: a VL, and a CL domain.

In some embodiments, the VH and the VL associate with each other, forming an antigen binding site of a Fab that specifically binds to a T cell antigen. In some embodiments, the first VHH specifically binds to a first tumor-associated antigen and the second VHH specifically binds to a second tumor-associated antigen.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

In some embodiments, the T cell antigen is CD3, and the first tumor-associated antigen is selected from the group consisting of: CEA, CEA-CAM6, HER2 and EGFR.

In some embodiments, the T cell antigen is CD3, and the second tumor-associated antigen is selected from the group consisting of: CEA, CEA-CAM6, HER2 and EGFR.

In one aspect, the disclosure is related to a protein complex, comprising:
(a) a first polypeptide comprising in the direction of N-terminus to C-terminus: optionally a first hinge region, a first CH2 domain, and a first CH3 domain, and a first VHH;
(b) a second polypeptide comprising in the direction of N-terminus to C-terminus: a VH, a CH1 domain, optionally a second hinge region, a second CH2 domain, a second CH3 domain, and a second VHH; and
(c) a third polypeptide comprising in the direction of N-terminus to C-terminus: a VL, and a CL domain.

In some embodiments, the VH and the VL associate with each other, forming an antigen binding site of a Fab that specifically binds to a T cell antigen. In some embodiments, the first VHH specifically binds to a first tumor-associated antigen and the second VHH specifically binds to a second tumor-associated antigen.

In some embodiments, the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

In some embodiments, the T cell antigen is CD3, and the first tumor-associated antigen is selected from the group consisting of: CEA, CEA-CAM6, HER2 and EGFR.

In some embodiments, the T cell antigen is CD3, and the second tumor-associated antigen is selected from the group consisting of: CEA, CEA-CAM6, HER2 and EGFR.

In some embodiments, the first CH3 domain comprises one or more knob mutations, and the second CH3 domain comprises one or more hole mutations.

In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NO: 6.

In some embodiments, the VHH comprises a sequence that is at least 80%, 90%, 95%, or 100% identical to any one of SEQ ID NOs: 7-10.

In some embodiments, the VHH is linked to the CH3 domain via a linker peptide.

In one aspect, the disclosure is related to a nucleic acid comprising a polynucleotide encoding the antigen-binding protein described herein, or the protein complex described herein.

In some embodiments, the nucleic acid is a DNA (e.g., cDNA) or RNA (e.g., mRNA).

In one aspect, the disclosure is related to a vector comprising one or more of the nucleic acids described herein.

In one aspect, the disclosure is related to a cell comprising the vector described herein.

In some embodiments, the cell is a HEK293F cell or CHO cell.

In one aspect, the disclosure is related to a cell comprising one or more of the nucleic acids described herein.

In one aspect, the disclosure is related to a method of producing an antigen-binding protein or protein complex, the method comprising culturing the cell described herein under conditions sufficient for the cell to produce the antigen-binding protein or protein complex; and collecting the antigen-binding protein or protein complex produced by the cell.

In one aspect, the disclosure is related to an antibody-drug conjugate comprising the antigen-binding protein described herein, or the protein complex described herein, covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antigen-binding protein described herein, the protein complex described herein, or the antibody-drug conjugate described herein, to the subject.

In some embodiments, the subject has a cancer expressing CEA-CAM6.

In some embodiments, the cancer is lung cancer, colorectal cancer, head and neck cancer, stomach cancer, pancreatic cancer, urothelial cancer, breast cancer, cervical cancer, or endometrial cancer.

In one aspect, the disclosure is related to a method of decreasing the rate of tumor growth, the method comprising contacting a tumor cell with an effective amount of a composition comprising the antigen-binding protein described herein, the protein complex described herein, or the antibody-drug conjugate described herein.

In one aspect, the disclosure is related to a method of killing a tumor cell, the method comprising contacting a tumor cell with an effective amount of a composition comprising the antigen-binding protein described herein, the protein complex described herein, or the antibody-drug conjugate described herein.

In one aspect, the disclosure is related to a pharmaceutical composition comprising the antigen-binding protein described herein, or the protein complex described herein, and a pharmaceutically acceptable carrier.

In one aspect, the disclosure provides an antigen-binding protein, comprising (a) a Fc; (b) a Fab fragment (Fab) that specifically binds to a T cell antigen; and (c) a single-domain antibody variable domain (VHH) that specifically binds to a tumor-associated antigen.

In some embodiments, the Fab and the VHH are linked to the Fc. In some embodiments, the Fab comprises or consists of a light chain variable domain (VL), a light chain constant domain (CL), a heavy chain variable domain (VH), and a heavy chain first constant domain (CH1). In some embodiments, the Fab can activate T cells upon binding to the T cell antigen. In some embodiments, the T cell antigen is cluster of differentiation 3 (CD3).

In some embodiments, the tumor-associated antigen is cluster of differentiate 20 (CD20), carcinoembryonic antigen (CEA), prostate-specific antigen (PSA), prostate stem cell antigen (PSCA), programmed death-ligand 1 (PD-L1), human epidermal growth factor receptor 2 (HER2), human epidermal growth factor receptor 3 (HER3), human epidermal growth factor receptor 1 (HER1), β-Catenin, cluster of differentiate 19 (CD19), epidermal growth factor receptor (EGFR), tyrosine-protein kinase Met (c-Met), epithelial cell adhesion molecule (EPCAM), prostate-specific membrane antigen (PSMA), cluster of differentiate 40 (CD40), Mucin 1, Cell Surface Associated (MUC1), insulin-like growth factor 1 receptor (IGF1R), or carcinoembryonic antigen cell adhesion molecule 6 (CEA-CAM6).

In some embodiments, the Fe is human IgG4 Fe. In some embodiments, the CH1 domain of the Fab is linked to a CH2 domain in the Fe, optionally via a hinge region. In some embodiments, the hinge region is a human IgG4 hinge region optionally with S228P mutation according to EU numbering. In some embodiments, the Fab is linked to the C-terminus of a CH3 domain in the Fe. In some embodiments, the Fab is linked to the CH3 domain via a linker peptide. In some embodiments, the VHH is linked to a CH2 domain in the Fe, optionally via a hinge region. In some embodiments, the hinge region is a human IgG4 hinge region optionally with S228P mutation according to EU numbering. In some embodiments, the VHH is linked to the C-terminus of a CH3 domain in the Fe. In some embodiments, the VHH is linked to the CH3 domain via a linker peptide. In some embodiments, the Fc comprises a first polypeptide and a second polypeptide. In some embodiments, each polypeptide comprises one or more knobs-into-holes mutations.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multispecific antibodies, bispecific antibodies, trispecific antibodies, single-chain antibodies, diabodies, linear antibodies formed from these antibodies or antibody fragments, and antigen binding protein constructs.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)₂, and Fv fragments, scFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to CEA-CAM6 may be referred to as CEA-CAM6-specific antibody or an anti-CEA-CAM6 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody.

In some embodiments, the multispecific antibody binds to two, three, four, five, or six different epitopes.

As used herein, a “VHH” refers to the variable domain of a heavy chain antibody. In some embodiments, the VHH is a humanized VHH. In some embodiments, the VHH is a single-domain antibody (sdAb).

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

As used herein, an “unilateral” multispecific antibody refers to a multispecific antibody where all antigen-binding domains are located on the same side of the Fc region (either at the N-terminus of the Fc region or at the C-terminus of the Fe region). In some embodiments, the unilateral multispecific antibody is a unilateral bispecific antibody or a unilateral trispecific antibody.

As used herein, a “non-unilateral” multispecific antibody refers to a multispecific antibody where not all antigen-binding domains are located on the same side of the Fe region (either at the N-terminus of the Fc region or at the C-terminus of the Fe region). In some embodiments, the non-unilateral multispecific antibody is a non-unilateral bispecific antibody or a non-unilateral trispecific antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D show schematic structures of several bispecific antibody constructs (CEA-BsAb, EGFR-BsAb, HER2-BsAb and CEACAM6-BsAb) including a Fab (T cell activator) and a tumor-associated antigen-targeting moiety in the form of sdAb.

FIGS. 2A-2L show schematic structures of several non-unilateral trispecific antibody constructs (301-312) including (1) a Fab (T cell activator), (2) a tumor-associated antigen-targeting moiety in the form of sdAb and (3) another tumor-associated antigen-targeting moiety in the form of sdAb.

FIGS. 3A-3B show images of SDS-PAGE (sodium dodecyl sulfate-polyacrylamide gel electrophoresis) results of the trispecific antibodies (301-312). Each trispecific antibody sample was analyzed under either a non-reducing or reducing condition. MK is the molecular weight (MW) marker. Names of the trispecific antibodies are labeled over each gel image. Band sizes are also labeled on the left side of each gel image.

FIGS. 4A-4F show the T cell mediated cell killing effects of target tumor cells induced by the non-unilateral CD3/CEA/EGFR trispecific antibodies. The following tumor target cells were tested: NCI-H157 cells (FIGS. 4A-4B), NCI-H1299 cells (FIGS. 4C-4D), and NCI-H1395 cells (FIGS. 4E-4F).

FIGS. 5A-5N show the cytokine secretion (IL-2, IL-6, IFN-γ and TNF-α) after T cell mediated killing of tumor cells (NCI-H157) induced by CD3/CEA/EGFR trispecific antibodies (301-312).

FIGS. 6A-6N show the cytokine secretion (IL-2, IL-6, IFN-γ and TNF-α) after T cell mediated killing of tumor cells (NCI-H1299) induced by CD3/CEA/EGFR trispecific antibodies (301-312).

FIGS. 7A-7N show the cytokine secretion (IL-2, IL-6, IFN-γ and TNF-α) after T cell mediated killing of tumor cells (NCI-H1395) induced by CD3/CEA/EGFR trispecific antibodies (301-312).

FIGS. 8A-8P show schematic structures of several unilateral trispecific antibody constructs (401-416) including (1) a Fab (T cell activator), (2) a tumor-associated antigen-targeting moiety in the form of sdAb and (3) another tumor-associated antigen-targeting moiety in the form of sdAb.

FIGS. 9A-9B show images of SDS-PAGE results of the trispecific antibodies (401-416). Each trispecific antibody sample was analyzed under either a non-reducing or reducing condition. MK is the molecular weight (MW) marker. Names of the trispecific antibodies are labeled over each gel image. Band sizes are also labeled on the left side of each gel image.

FIGS. 10A-10F show the T cell mediated cell killing effects of target tumor cells induced by the unilateral trispecific antibodies (401-416). The following tumor target cells were tested: NCI-H157 cells (FIGS. 10A-10B), NCI-H1299 cells (FIGS. 10C-10D), NCI-H1395 cells (FIGS. 10E-10F), NCI-H1573 (FIGS. 10G-10H), NCI-H157-21# (FIG. 10I) and SK-BR-3 (FIG. 10J).

FIGS. 11A-11R show schematic structures of several unilateral trispecific antibody constructs (501, 503, 509-512, 601, 603, 609-612, 701, 703, 709-712) including (1) a Fab (T cell activator), (2) a tumor-associated antigen-targeting moiety in the form of sdAb and (3) another tumor-associated antigen-targeting moiety in the form of sdAb.

FIGS. 12A-12B show images of SDS-PAGE results of the trispecific antibodies (501, 503, 509-512, 601, 603, 609-612, 701, 703, 709-712). Each trispecific antibody sample was analyzed under either a non-reducing or reducing condition. MK is the molecular weight (MW) marker. Names of the trispecific antibodies are labeled over each gel image. Band sizes are also labeled on the left side of each gel image.

FIGS. 13A-13F show the T cell mediated cell killing effects of target tumor cells induced by the unilateral trispecific antibodies (501, 503, 509-512, 601, 603, 609-612, 701, 703, 709-712). The following tumor target cells were tested: NCI-H1975 cells (FIG. 13A), MDA-MB-468 cells (FIG. 13B), NCI-H157-21# cells (FIG. 13C), SK-OV-3 cells (FIGS. 13D-13F), NCI-H1573 cells (FIGS. 13G-131), MCF-7 cells (FIGS. 13J-13K), MKN-45 cells (FIGS. 13L-13N), SK-BR-3 cells (FIGS. 130-13Q) and MDA-MB-453 cells (FIGS. 13R-13T).

FIG. 14 shows the schematic structure of a control bispecific antibody.

FIGS. 15A-15L show the schematic structures of several unilateral trispecific antibody constructs (801-809, 813-815) including (1) a Fab (T cell activator), (2) a tumor-associated antigen-targeting moiety in the form of sdAb and (3) another tumor-associated antigen-targeting moiety in the form of sdAb.

FIGS. 16A-16B show images of SDS-PAGE results of the trispecific antibodies (801-809, 813-815). Each trispecific antibody sample was analyzed under either a non-reducing or reducing condition. M is the molecular weight (MW) marker. Names of the trispecific antibodies are labeled over each gel image. Band sizes are also labeled on the left side of each gel image.

FIGS. 17A-17H show the in vivo anti-tumor effects of the unilateral trispecific antibodies (801-809, 813-815) in mice. FIGS. 17A-17D show tumor size data and FIGS. 17E-17H show body weight of the mice.

### DETAILED DESCRIPTION

This disclosure relates to multispecific antibodies (e.g., trispecific antibodies) or antigen-binding proteins. In one aspect, the multispecific antibodies or antigen-binding proteins can bind to a T cell antigen (e.g., CD3) and/or one or more tumor-associated antigens (e.g., CEA, CEA-CAM6, EGFR, HER2), or a combination thereof.

Cluster of differentiation 3 (CD3) is known in the art as a multi-protein complex of six chains (see, Abbas and Lichtman, 2003; Janeway et al., p 172 and 178, 1999). In mammals, the complex comprises a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. It is believed the ITAMs are important for the signaling capacity of a TCR complex.

Carcinoembryonic antigen (CEA) is a substance found on the surface of some cells. It is a type of glycoprotein produced by cells of the gastrointestinal tract during embryonic development. It is produced in very small amounts after birth. The level of CEA in the bloodstream is thus relatively low unless certain diseases including certain forms of cancer are present. In the past, CEA was considered as an oncofetal antigen, expressed during the embryonic development and re-expressed only in cancer patients. Indeed, CEA is also expressed in normal adults but it is produced in the colon and then disappears in feces. This cell surface glycoprotein plays a role in cell adhesion and in intracellular signaling. In colon cancer, the tumor cells have lost their polarity and CEA is distributed around the cell surface and eventually gets into the blood. CEA is a complex, highly glycosylated macromolecule (50% carbohydrates) with a molecular weight of ˜200 kDa, compared to 160 kDa for well-known glucose oxidase. Indeed, CEA in normal colon tissues has a broad band averaging at 200 kDa and a sharp band at 130 kDa. However, in cancer cells, only a single band at 170 kDa or lower has been observed, which is partly due to the modification of the glycosylation pattern of CEA by N-glycanase. As a stable molecule, CEA is one of the most widely used tumor biomarkers and a prognostic indicator in clinical assays. CEA should be minimal in the blood of healthy adults whereas an abnormal level of CEA may be a sign of cancer, especially colon and rectal cancer. Serum from patients with colorectal carcinoma often has higher CEA level than healthy individuals, ˜2.5 μg/L. CEA may also be present in patients with pancreas, liver, breast, ovary, or lung cancer. Therefore, the CEA level measured before and after surgery indicates the surgical success and the prognosis of patient's recovery. CEA levels may also be measured during chemotherapy to evaluate the treatment progress and outcome.

The epidermal growth factor receptor (EGFR) belongs to the ErbB family of receptor tyrosine kinases (RTKs) and exerts critical functions in epithelial cell physiology (Schlessinger, 2014). It is frequently mutated and/or overexpressed in different types of human cancers and is the target of multiple cancer therapies currently adopted in the clinical practice (Yarden and Pines, 2012). The physiological function of the epidermal growth factor receptor (EGFR) is to regulate epithelial tissue development and homeostasis. In pathological settings, mostly in lung and breast cancer and in glioblastoma, the EGFR is a driver of tumorigenesis. Inappropriate activation of the EGFR in cancer mainly results from amplification and point mutations at the genomic locus, but transcriptional upregulation or ligand overproduction due to autocrine/paracrine mechanisms has also been described. Moreover, the EGFR is increasingly recognized as a biomarker of resistance in tumors, as its amplification or secondary mutations have been found to arise under drug pressure. This evidence, in addition to the prominent function that this receptor plays in normal epithelia, has prompted intense investigations into the role of the EGFR both at physiological and at pathological level.

Human epidermal growth factor receptor 2 (HER2) is a transmembrane growth factor receptor found in normal and malignant breast epithelial cells. Phosphorylation of the intracellular tyrosine kinase results in intracellular signaling and activation of genes involved in cell growth. Overexpression of HER2 has independent prognostic significance in early breast cancer and may also predict response to hormonal and cytotoxic therapies, although this latter role is less well studied. Prospective stratification of HER2 status in current clinical trials may more accurately delineate these roles. Anti-HER2 therapy, using a humanized monoclonal antibody, has enhanced survival when given with chemotherapy compared with chemotherapy alone in patients with metastatic HER2-overexpressing breast cancer. A potential limitation to its use in the adjuvant setting is the increased incidence of cardiotoxicity in patients treated either concurrently or previously with anthracyclines; carefully designed prospective adjuvant trials are currently being launched. HER2 is a relatively new prognostic marker and holds promise for predicting response to various therapies and for target-specific therapy.

Carcinoembryonic antigen (CEA)-related cell adhesion molecule 6 (CEACAM6) is a glycophosphoinositol-anchored glycoprotein and belongs to the CEA family. Much evidence has demonstrated that CEACAM6 expression is upregulated in gastrointestinal tumors and promotes gastrointestinal cancer progression through multiple mechanisms. However, the role of CEACAM6 in nongastrointestinal cancer cells is still controversial. For example, CEACAM6 was used as a marker for isolating cancer stem cells in colorectal cancer, while the CEACAM6-positive subpopulation of breast cancer cells showed less sphere formation and less tumorigenesis in nude mice compared to CEACAM6-negative ones. The discrepancies are currently unresolved, but it is suspected that post-translational glycosylation of CEACAM6 may be critical because glycosylation is a cell type-dependent process. The CEACAM6 polypeptide contains 344 amino acids with a predictive molecular weight (MW) of 37 kDa, but multiple glycosylated isoforms have been reported in different kinds of tissues and in different development stages. CEACAM6 is a heavily glycosylated tumor-associated molecule with an MW ranging 75˜100 kDa in pancreatic and colorectal carcinoma. In addition, biochemical studies showed that the N-glycosylation of CEACAM immunoglobulin V (IgV) domains affects their oligomerization or dimerization. CEACAM5 glycans in colorectal cancer cells interacted with antigen-presenting cells by glycan-binding proteins such as DC-SIGN (a human C-type lectin).17 The results suggested that glycosylation could regulate the protein structure and biological functions of CEACAM6.

In general, multispecific antibodies (e.g., trispecific antibodies) include two or more antigen-binding sites targeting different antigens or different epitopes of the same antigen. Thus, multispecific antibodies (e.g., trispecific antibodies) can have more functions than a monospecific antibody. For example, these functions include, but not limited to, stronger binding to an antigen through an avidity effect; co-localization of bound antigens on the cell surface and the effect therefrom; increasing the serum half-life of an antibody fragment by linking it to a second antibody fragment that is bound to a protein with a long serum half-life, e.g., albumin or transferrin; and bringing two cells into proximity by binding to an antigen on each of the cells.

Among the purposes of multispecific antibodies (e.g., trispecific antibodies), one class of molecules, T cell engagers (TCE), has gained more attention. A TCE is a multispecific antibody (e.g., trispecific antibodies) which binds to an antigen on a T cell and an antigen on another cell simultaneously. CD3 is usually selected as the antigen on the T cell. A cancer or tumor cell is usually selected as the other cell type as discussed above. Through binding to CD3 on T cells and a tumor associated antigen (TAA) on cancer cells, the TCE can induce activation of T cells upon binding to cancer cells and cause the killing of the latter.

Nevertheless, there are some hurdles to overcome in order to generate desirable homogeneous multispecific antibodies (e.g., trispecific antibodies). The first hurdle is mismatch of heavy chains that bind to the same target (e.g., antigen or epitope). For example, to generate multispecific antibodies with a desired format, the heavy chains targeting different targets should ideally form a heterodimer. However, the percentage of the desired multispecific antibodies varies greatly in different constructs. Mutations to induce the formation of knobs-into-holes between two heavy chains can be employed to reduce the formation of homodimers of the heavy chains that bind to the same target. Exemplary amino acid sequences of knob-chain and hole-chain Fc that facilitate heterodimer formation are set forth in SEQ ID NO: 1 and SEQ ID NO: 2, respectively.

The second hurdle to overcome is the mismatch between heavy chain variable regions (VHs) and light chain variable regions (VLs). A monoclonal antibody has two identical Fab fragments, each having a paired VH and VL. By contrast, a multispecific antibody usually has several different heavy chain variable regions and several different light chain variable regions. Therefore, there is a possibility that each VH can bind to multiple VLs and each VL can bind to multiple VHs. As a result, only some of the formed multispecific antibodies are functional without addressing this mismatch issue.

Several strategies have been designed to disable this mismatch. One solution is to design an antibody with a common light chain. Specifically, a light chain, or more precisely a VL, is selected which can form a dimer with all VHs. This design abrogated the necessity of matching VH and VL with the same target-binding specificity. The shortcoming of this strategy is that the contribution of VL in target binding is greatly reduced, leading to difficulty finding an optimal VH as the VH will be greatly if not entirely responsible for target binding.

Another solution is the use of CrossMAb technology, in which a VL is fused to heavy chain constant domain 1 (CH1) and becomes part of this heavy chain. Meanwhile, the corresponding VH is fused to the light chain constant region (CL) and becomes a part of this light chain.

The present disclosure provides a different strategy. The reason of employing technologies, e.g., CrossMAb or common light chain, is because conventional antibodies have and need both heavy and light chains to function and/or maintain stability. However, target binding does not necessarily require both heavy and light chains. For example, a Fab fragment (Fab) contains a variable and constant domain of the light chain and a variable domain and the first constant domain (CH1) of the heavy chain, is fully functional for antigen binding. The variable domain of heavy chain antibodies (VHH), e.g., derived from camelids such as llama, camel, or alpaca, is fully functional for antigen binding. By fusing antibody fragments (e.g., Fab or VHH) to human Fc with heterodimer preference, multispecific antibodies can be generated without considering the mismatch between heavy and light chains.

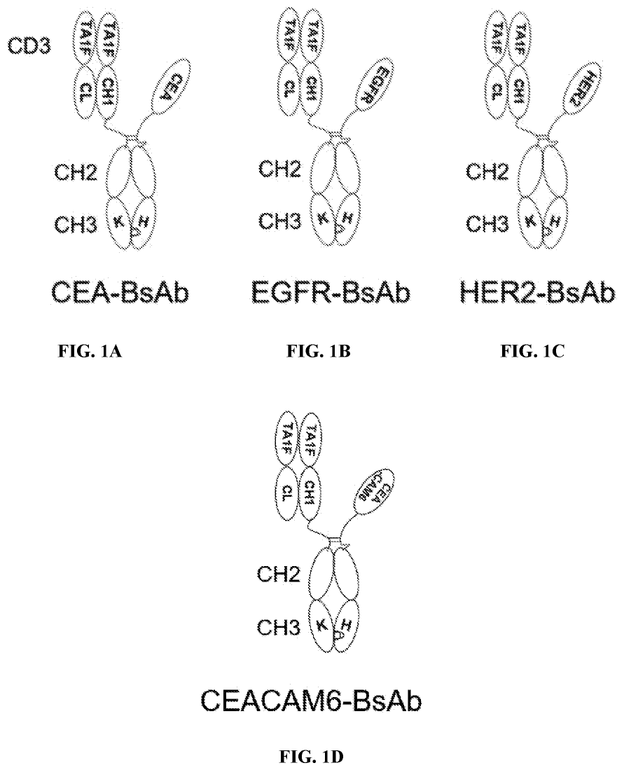

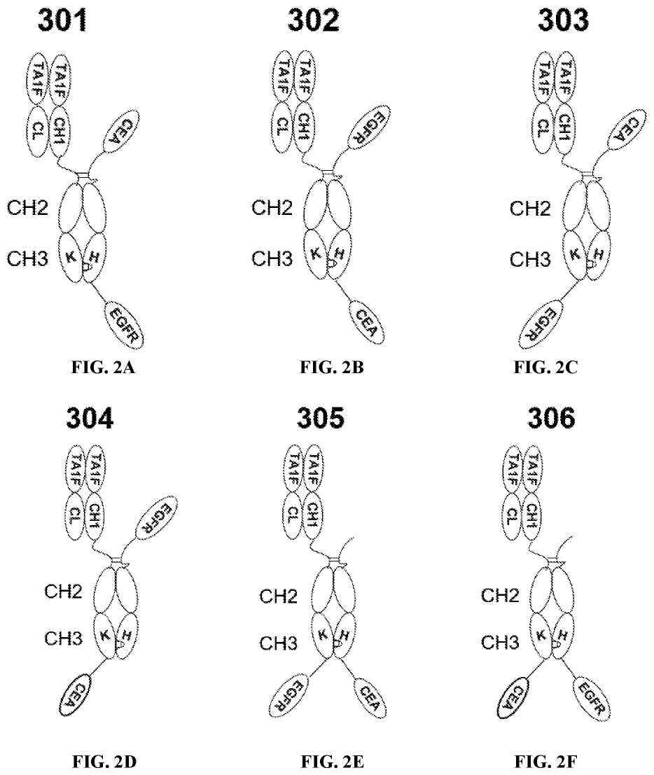

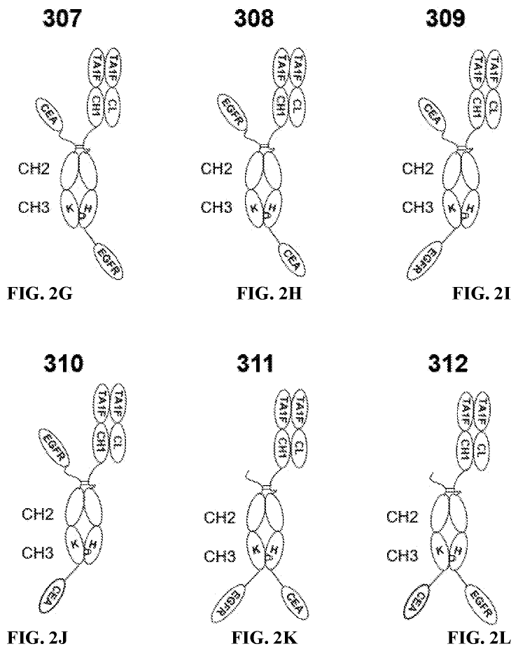

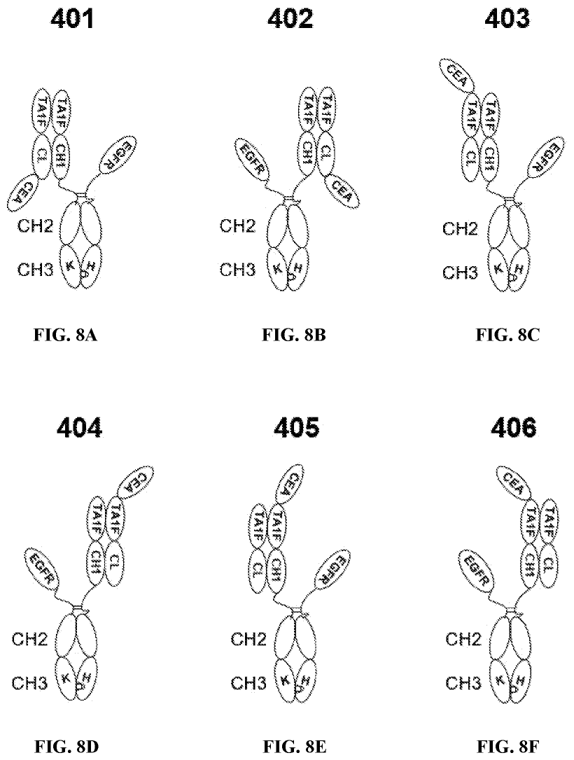

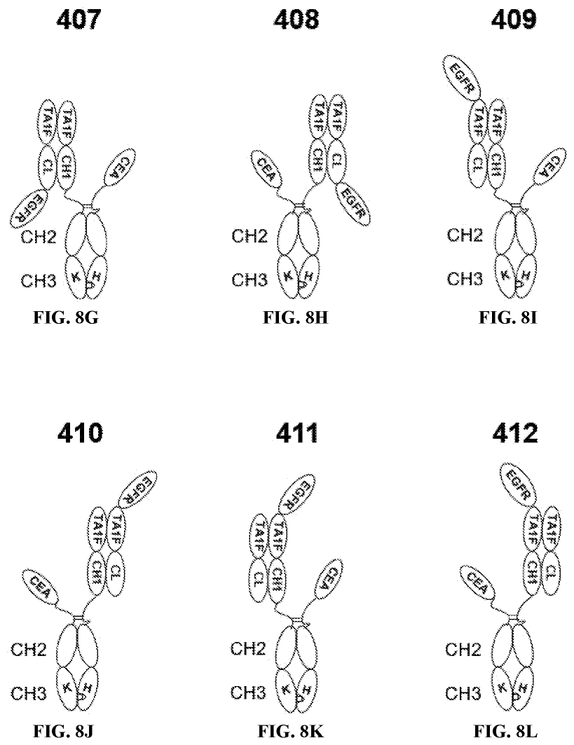

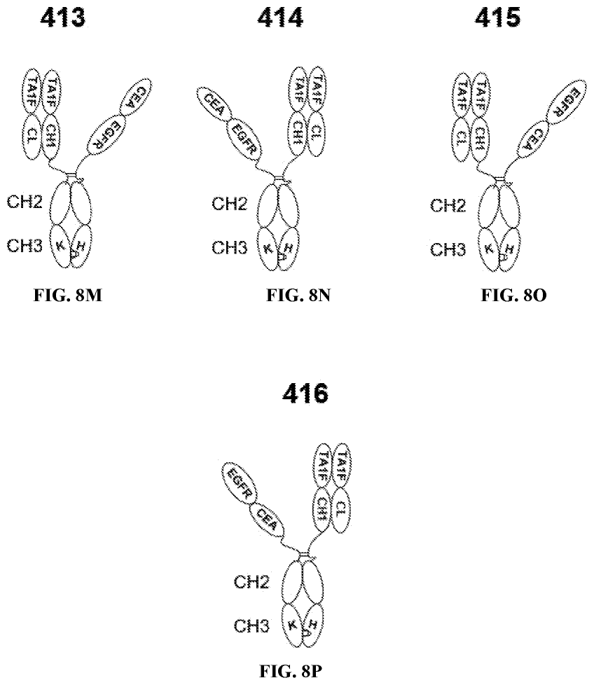

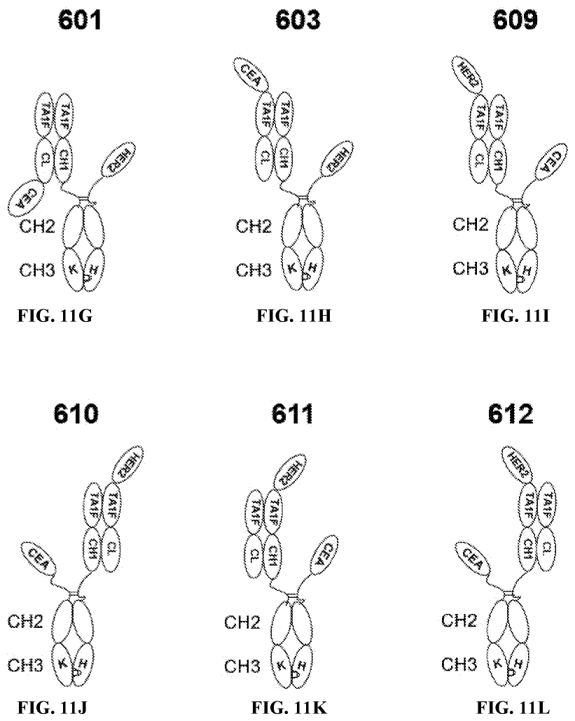

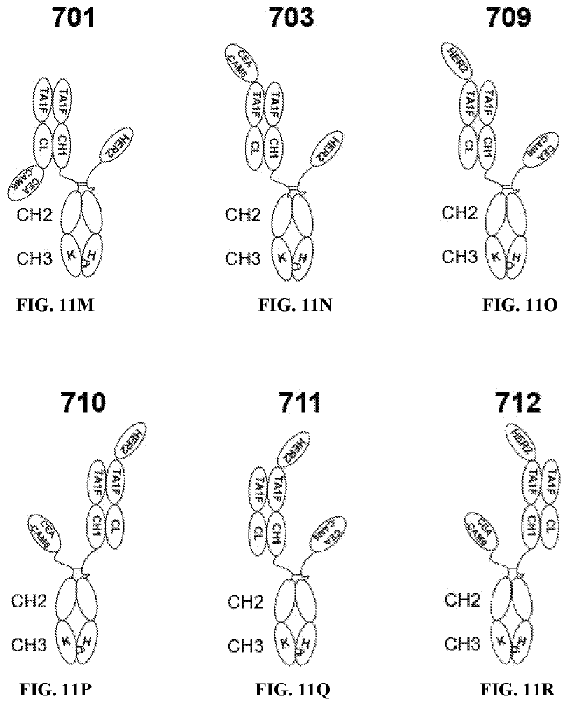

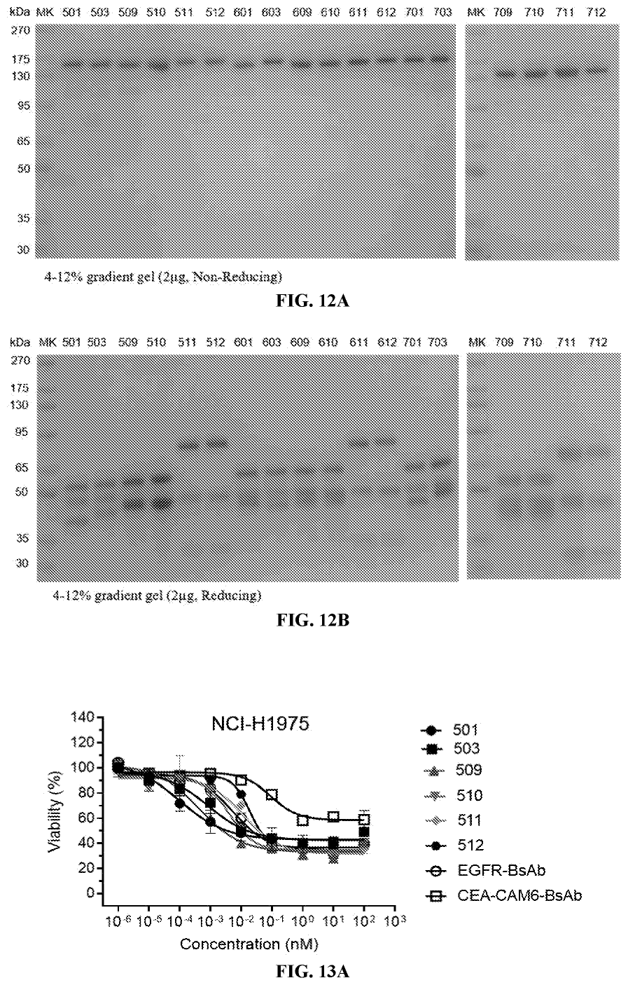

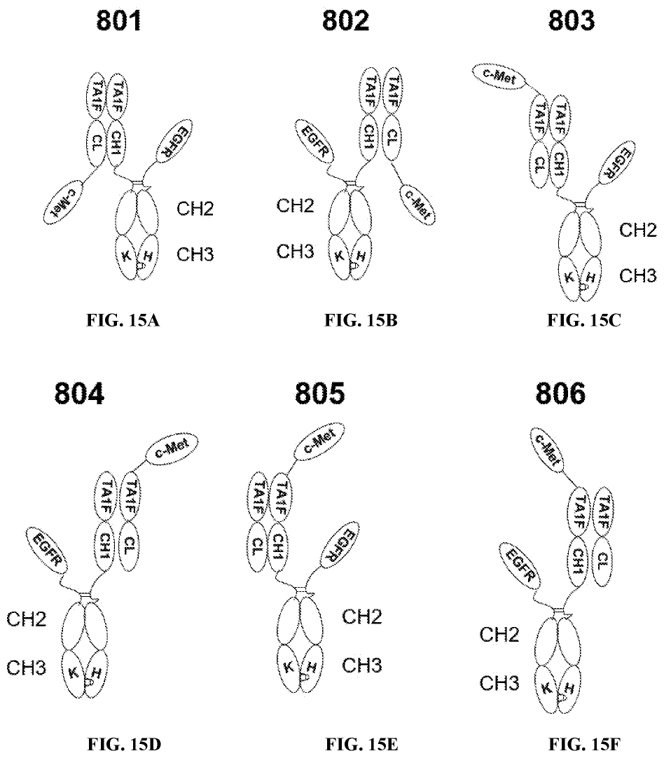

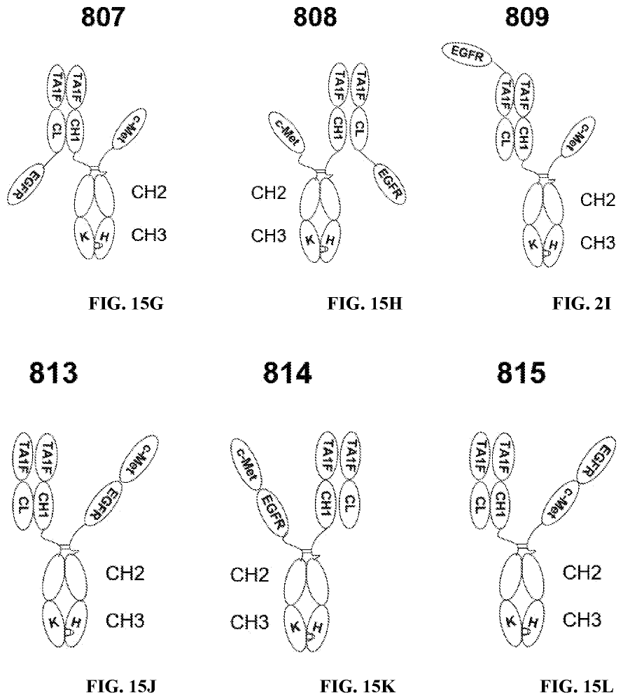

In this disclosure, a Fab that can bind to human CD3 and a VHH that can bind to one or more tumor associated antigens (e.g., HER2, CEA-CAM6, or EGFR) is provided. Either antibody fragment is positioned on each of the four ends of Fe (N- or C-end of knob or hole chain), and the other antibody fragment on the other three available ends of the Fe. Such a design gives rise to 4 different bispecific antibody molecules (See FIGS. 1A-1D), and 12 different non-unilateral trispecific antibodies (See FIGS. 2A-2L, corresponding to 301-312). Further, 16 different unilateral trispecific antibodies were constructed where the CD3 Fab and two VHHs are located on the same side of the Fe region (See FIGS. 8A-8P, corresponding to 401-416). Using different VHHs, additional 18 unilateral trispecific antibodies were constructed (See FIGS. 11A-11R, corresponding to 501, 503, 509-512, 601, 603, 609-612, 701, 703, 709-712). Using different VHHs, additional 12 unilateral trispecific antibodies were constructed (See FIGS. 15A-15L, corresponding to 801-809, 813-815). These molecules are provided, characterized for their ability to form heterodimer, and tested for their capability in inducing T cell mediated tumor cell killing in the presence of human PBMCs.

In some embodiments, the multispecific antibody or antigen-binding protein can include 1, 2, 3, 4 or more than four Fab fragments. In some embodiments, the multispecific antibody or antigen-binding protein can include 1, 2, 3, 4, 5 or more than five VHHs. In some embodiments, the Fab can target CD3 or another tumor associated antigen. In some embodiments, the VHH can target CD3 or another tumor associated antigen. The Fab, the VHH, and the multispecific antibody or the antigen binding proteins with various formats are described in detail below.

### Fab Fragment (Fab)

A Fab fragment (Fab) contains a variable and constant domain of the light chain and a variable domain and the first constant domain (CH1) of the heavy chain.

The disclosure provides e.g., anti-CD3 antibodies, the modified antibodies thereof, the chimeric antibodies thereof, and the humanized antibodies thereof. The disclosure also provides Fab fragments that targets CD3. The Fab can be used in various multispecific antibody constructs as described herein.

In some embodiments, the Fab can have a VH that is at least 80%, 85%, 90%, 91%, 92%, 93%, 94%, 95%, 96%, 97%, 98%, or 99% identical to SEQ ID NO: 5 (TA1). In some embodiments, the Fab can have a VL that is at least 80%, 85%, 90%, 91%, 92%, 93%, 94%, 95%, 96%, 97%, 98%, or 99% identical to SEQ ID NO: 6 (TA1).

In some embodiments, the Fab, the antibody or an antigen-binding fragment described herein can have a heavy chain variable region (VH) comprising VH CDR1, VH CDR2, and VH CDR3 that are identical to VH CDR1, VH CDR2, and VH CDR3 of any VH as described herein (e.g., SEQ ID NO: 5); and a light chain variable region (VL) comprising VL CDR1, VL CDR2, and VL CDR3 that are identical to VL CDR1, VL CDR2, and VL CDR3 of a VL as described herein (e.g., SEQ ID NO: 6).

In some embodiments, the Fab, the antibody or an antigen-binding fragment described herein can contain a VH containing VH CDR1 with zero, one or two amino acid insertions, deletions, or substitutions; VH CDR2 with zero, one or two amino acid insertions, deletions, or substitutions; VH CDR3 with zero, one or two amino acid insertions, deletions, or substitutions, and a VL containing one, two, or three of VL CDR1 with zero, one or two amino acid insertions, deletions, or substitutions; VL CDR2 with zero, one or two amino acid insertions, deletions, or substitutions; VL CDR3 with zero, one or two amino acid insertions, deletions, or substitutions, wherein the VH CDRs are selected from any VH as described herein, and the VL CDRs are selected from any VL as described herein.

### Heavy-Chain Antibody Variable Domain (VHH)

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

The disclosure provides e.g., anti-CEA-CAM6 antibodies, the modified antibodies thereof, the chimeric antibodies thereof, and the humanized antibodies thereof. The disclosure also provides VHH of these antibodies. These VHHs can be used in various multispecific antibody constructs as described herein.

The amino acid sequences for various VHH are also provided. In some embodiments, the VHH domain is at least 80%, 85%, 90%, 91%, 92%, 93%, 94%, 95%, 96%, 97%, 98%, or 99% identical to any one of SEQ ID NOs: 7-10.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs of any one of SEQ ID NOs: 7-10.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished, e.g., using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy-chain antibody variable domain (VHH).

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

### Structures of Multispecific Antibodies

The multispecific antibodies (e.g., bispecific antibodies or trispecific antibodies) can be designed to include one or more antigen-binding sites that target T cell antigens (e.g., CD3, CD4, and CD8), and include one or more antigen-binding sites that target a tumor-associated antigen (e.g., CEA, CEA-CAM6, HER2, EGFR). The antigen-binding site can comprise e.g., a Fab, a scFv, a VHH. In some embodiments, the one or more antigen-binding sites that target a T cell antigen (e.g., CD3) can comprise a Fab. In some embodiments, the one or more antigen-binding sites that target the tumor-associated antigen can comprise a VHH. The tumor-associated antigen refers to an antigen that is specifically expressed on tumor cell surfaces. These antigens can be used to identify tumor cells. Normal cells rarely express these tumor associated antigens. Some exemplary tumor-associated antigens include, e.g., CEA, CD20, PSA, PSCA, PD-L1, HER2, Her3, Her1, β-Catenin, CD19, CEA-CAM6, EGFR, c-Met, EPCAM, PSMA, CD40, MUC1, and IGF1R, etc.

In some embodiments, a multispecific antibody (e.g., bispecific antibody or trispecific antibody) or antigen-binding fragment thereof described herein includes a Fab that specifically binds to a T cell antigen. In some embodiments, the T cell antigen is CD3 (e.g., human CD3). In some embodiments, the T cell antigen is CD28. In some embodiments, the T cell antigen is CD27. In some embodiments, the T cell antigen is CD137. In some embodiments, the T cell antigen is OX40. In some embodiments, the T cell antigen is PD1. In some embodiments, the T cell antigen is CTLA-4. In some embodiments, the T cell antigen is Tim3. In some embodiments, the T cell antigen is LAG-3. In some embodiments, the multispecific antibody or antigen-binding fragment thereof can activate T cells upon binding to the T cell antigen.

In some embodiments, a multispecific antibody (e.g., bispecific antibody or trispecific antibody) or antigen-binding fragment thereof described herein includes a VHH that specifically binds to a tumor-associated antigen. In some embodiments, the tumor-associated antigen is CEA-CAM6 (e.g., human CEA-CAM6). In some embodiments, the tumor-associated antigen is CEA. In some embodiments, the tumor-associated antigen is EGFR or HER2. In some embodiments, the tumor-associated antigen is Claudin18.2. In some embodiments, the tumor-associated antigen is CD166. In some embodiments, the tumor-associated antigen is Glypican-3. These are just examples of tumor-associated antigens. Listing of them does not mean to limit the utility of only these antigens.

The present disclosure provides antigen-binding protein constructs with various formats as described herein. While not intending to be bound by any theory, it is hypothesized that the in the presence of the target cells (e.g., cancer cells) and T cells, the protein constructs can effectively activate T cells.

In some embodiments, the multispecific antibodies (e.g., bispecific antibodies or trispecific antibodies) are designed to include a Fab that targets CD3. In some embodiments, the multispecific antibodies (e.g., bispecific antibodies or trispecific antibodies) are designed to include a VHH that targets CEA-CAM6. The multispecific antibodies are described below.

CD3 (cluster of differentiation 3) is a protein complex and T cell co-receptor that is involved in activating both the cytotoxic T cell (CD8+ naive T cells) and T helper cells (CD4+ naive T cells). It is composed of four distinct chains. In mammals, the complex contains CD3γ chain, CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex. In some embodiments, the multispecific antibodies target CD3ε.

CEA-CAM6 (carcinoembryonic antigen-related cell adhesion molecule 6) is a cell surface glycoprotein that is a member of the carcinoembryonic antigen (CEA) family of proteins. Members of this family play a role in cell adhesion and are widely used as tumor markers in serum immunoassay determinations of carcinoma.

The present disclosure provides multispecific antibodies (e.g., bispecific antibodies or trispecific antibodies) that bind to both a T cell antigen (e.g., CD3) and a tumor associated antigen. The multispecific antibodies can be used to treat tumor associated antigen positive cancers in a subject (e.g., a human patient). In some embodiments, the tumor associated antigen positive cancer is CEA-CAM6-positive (e.g., ovarian, colon, breast or non-small cell lung cancers).

In general, the multispecific antibody (e.g., bispecific antibody or trispecific antibody) described herein can be prepared, which includes (a) a first polypeptide including a first Fc region (e.g., CH2 domain and CH3 domain); and (b) a second polypeptide including a second Fe region (e.g., CH2 domain and CH3 domain). In some embodiments, the first Fe region and/or the second Fe region are derived from human IgG4. In some embodiments, the first Fe region and/or the second Fe region include one or more knobs-into-holes mutations. For example, the first Fc region (e.g., the CH3 domain in the Fc region) can include a tryptophan (Trp) at position 366 according to EU numbering; and the second Fe region (e.g., the CH3 domain in the Fe region) can include one or more of the following a serine (Ser) at position 366, an alanine (Ala) at position 368, and/or a valine (Val) at position 407 according to EU numbering.

In some embodiments, the Fc region is derived from the Fe of any antibody as described herein (e.g., IgG1, IgG2, IgG3, and IgG4). In some embodiments, the Fc region is a human IgG1, IgG2, or IgG4 (e.g., a human IgG4). In some embodiments, the first Fe region and/or the second Fc region include additional mutations relative to the Fc region of a wild-type human IgG (e.g., IgG4). For example, the first Fc region and/or the second Fe region can include a proline (Pro) at position 228 according to EU numbering, to reduce chain exchange of the multispecific antibody. The first Fe region and/or the second Fc region can also include an alanine (Ala) at positions 234 according to EU numbering, to reduce ADCC effect of the multispecific antibody. The first Fc region can include a cysteine (Cys) at position 354 and the second Fc region can further include a cysteine (Cys) at position 349 according to EU numbering, to stabilize the multispecific antibody. The second Fc region can include a lysine (Lys) at position 435 and/or a phenylalanine (Phe) at position 436 according to EU numbering, to reduce binding of the second polypeptide to Protein A. In addition, to improve antibody stability, a glycine (Gly) at position 446 and/or a lysine (Lys) at position 447 of the first Fe region and/or the second Fe region can be deleted. While not intending to be bound by any theory, it is understood by a person skilled in the art that the mutations and deletions described herein can be introduced in either the first Fc region or the second Fc region.

In one aspect, the disclosure is related to an antigen-binding protein, comprising (a) a Fe; (b) a first antigen-binding site comprising a Fab that specifically binds to CD3; and (c) a second antigen-binding site comprising a single-domain antibody variable domain (VHH) that specifically binds to a tumor associated antigen, in some embodiments, the first antigen-binding site and the second antigen-binding site are linked to the Fc.

In some embodiments, the first antigen-binding site comprises a Fab fragment (Fab) that contains a variable and constant domain of the light chain and a variable domain and the first constant domain (CH1) of the heavy chain. In some embodiments, the Fab can activate T cells upon binding to the CD3. In some embodiments, the Fab is human IgG4 Fab.

In some embodiments, the Fab is linked to a CH2 domain in the Fc, optionally via a hinge region. In some embodiments, the hinge region is a human IgG4 hinge region optionally with S228P mutation according to EU numbering.

In some embodiments, the Fab is linked to the C-terminus of a CH3 domain in the Fc. In some embodiments, the Fab is linked to the CH3 domain via a linker peptide.

In some embodiments, the VHH is linked to a CH2 domain in the Fe, optionally via a hinge region. In some embodiments, the hinge region is a human IgG4 hinge region optionally with S228P mutation according to EU numbering.

In some embodiments, the VHH is linked to the C-terminus of a CH3 domain in the Fc. In some embodiments, the VHH is linked to the CH3 domain via a linker peptide.

In some embodiments, the Fc comprises a first polypeptide chain and a second polypeptide chain, in some embodiments, each chain comprises one or more knobs-into-holes mutations.

In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NO: 6.

In some embodiments, the Fc region comprise a sequence that is at least 80%, 90%, 95%, or 100% identical to SEQ ID NO: 1 or 2.

In some embodiments, the constant domain of the light chain comprises a sequence that is at least 80%, 90%, 95%, or 100% identical to SEQ ID NO: 3.

In some embodiments, the CH1 domain comprise a sequence that is at least 80%, 90%, 95%, or 100% identical to SEQ ID NO: 4.

In some embodiments, the linker peptide comprises a sequence that is at least 80%, 90%, 95%, or 100% identical to SEQ ID NO: 11.

The multispecific antibodies with various structures are described below.

### 1. Fab-Knob+VHH-Hole

As shown in FIGS. 1A-ID, a multispecific antibody (e.g., bispecific antibody) can be prepared, which includes (a) a first polypeptide including, preferably from N-terminus to C-terminus: a VH, a CH1 domain, optionally a first hinge region, and a first Fc region (e.g., CH2 domain and CH3 domain); (b) a second polypeptide including, preferably from N-terminus to C-terminus: a single-domain antibody variable domain (VHH), optionally a second hinge region, and a second Fe region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fe region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 2. Fab-Knob+VHH-Hole-VHH (301-302)

As shown in FIGS. 2A-2B (301-302), a multispecific antibody (e.g., trispecific antibody) can be prepared, which includes (a) a first polypeptide including, preferably from N-terminus to C-terminus: a VH, a CH1 domain, optionally a first hinge region, and a first Fc region (e.g., CH2 domain and CH3 domain); (b) a second polypeptide including, preferably from N-terminus to C-terminus: a first single-domain antibody variable domain (VHH), optionally a second hinge region, a second Fe region (e.g., CH2 domain and CH3 domain), optionally a linker peptide and a second VHH; and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fe region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 3. Fab-Knob-VHH+VHH-Hole (303-304)

As shown in FIGS. 2C-2D (303-304), a multispecific antibody (e.g., trispecific antibody) can be prepared, which includes (a) a first polypeptide including, preferably from N-terminus to C-terminus: a VH, a CH1 domain, optionally a first hinge region, a first Fc region (e.g., CH2 domain and CH3 domain), optionally a linker peptide and a first VHH; (b) a second polypeptide including, preferably from N-terminus to C-terminus: a second VHH, optionally a second hinge region, a second Fe region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 4. Fab-Knob-VHH+Hole-VHH (305-306)

As shown in FIGS. 2E-2F (305-306), a multispecific antibody (e.g., trispecific antibody) can be prepared, which includes (a) a first polypeptide including, preferably from N-terminus to C-terminus: a VH, a CH1 domain, optionally a first hinge region, a first Fc region (e.g., CH2 domain and CH3 domain), optionally a linker peptide and a first VHH; (b) a second polypeptide including, preferably from N-terminus to C-terminus: optionally a second hinge region, a second Fc region (e.g., CH2 domain and CH3 domain), optionally a linker peptide and a second VHH; and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fe region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 5. VHH-Knob+Fab-Hole-VHH (307-308)

As shown in FIGS. 2G-211 (307-308), a multispecific antibody (e.g., trispecific antibody) can be prepared, which includes (a) a first polypeptide including, preferably from N-terminus to C-terminus: a first VHH, optionally a first hinge region, a first Fc region (e.g., CH2 domain and CH3 domain); (b) a second polypeptide including, preferably from N-terminus to C-terminus: a VH, a CH1 domain, optionally a second hinge region, a second Fc region (e.g., CH2 domain and CH3 domain), optionally a linker peptide and a second VHH; and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fe region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 6. VHH-Knob-VHH+Fab-Hole (309-310)

As shown in FIGS. 2I-2G (309-310), a multispecific antibody (e.g., trispecific antibody) can be prepared, which includes (a) a first polypeptide including, preferably from N-terminus to C-terminus: a first VHH, optionally a first hinge region, a first Fc region (e.g., CH2 domain and CH3 domain), optionally a linker peptide and a second VHH; (b) a second polypeptide including, preferably from N-terminus to C-terminus: a VH, a CH1 domain, optionally a second hinge region, a second Fe region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 7. Knob-VHH+Fab-Hole-VHH (311-312)

As shown in FIGS. 2K-2L (311-312), a multispecific antibody (e.g., trispecific antibody) can be prepared, which includes (a) a first polypeptide including, preferably from N-terminus to C-terminus: optionally a first hinge region, a first Fc region (e.g., CH2 domain and CH3 domain), optionally a linker peptide and a first VHH; (b) a second polypeptide including, preferably from N-terminus to C-terminus: a VH, a CH1 domain, optionally a second hinge region, a second Fc region (e.g., CH2 domain and CH3 domain), optionally a linker peptide and a second VHH; and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 8. VHH-Fab-Knob+VHH-Hole (401, 407, 501, 601, 701, 801, 807)

As shown in FIGS. 8A (401), 8G (407), 11A (501), 11G (601), 11M (701), 15A (801), and 15G (807), a multispecific antibody (e.g., trispecific antibody) can be prepared, which includes (a) a first polypeptide including, preferably from N-terminus to C-terminus: a VH, a CH1 domain, optionally a first hinge region, a first Fc region (e.g., CH2 domain and CH3 domain); (b) a second polypeptide including, preferably from N-terminus to C-terminus: a first VHH, optionally a second hinge region, a second Fe region (e.g., CH2 domain and CH3 domain); and (c) a third polypeptide including, preferably from N-terminus to C-terminus: a VL, a CL, and a second VHH. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO: 2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 9. VHH-Knob+VHH-Fab-Hole (402, 408, 802, 808)

As shown in FIGS. 8B (402), 8H (408), 15B (802), and 15H (808), a multispecific antibody (e.g., trispecific antibody) can be prepared, which includes (a) a first polypeptide including, preferably from N-terminus to C-terminus: a first VHH, optionally a first hinge region, a first Fc region (e.g., CH2 domain and CH3 domain); (b) a second polypeptide including, preferably from N-terminus to C-terminus: a VH, a CH1 domain, optionally a second hinge region, a second Fc region (e.g., CH2 domain and CH3 domain); and (c) a third polypeptide including, preferably from N-terminus to C-terminus: a VL, a CL, and a second VHH. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 10. VHH-Fab-Knob+VHH-Hole (403, 409, 503, 509, 603, 609, 703, 709, 803, 809)

As shown in FIGS. 8C (403), 8I (409), 11B (503), 11C (509), 11H (603), 11I (609), 11N (703), 11O (709), 15C (803), and 15I (809), a multispecific antibody (e.g., trispecific antibody) can be prepared, which includes (a) a first polypeptide including, preferably from N-terminus to C-terminus: a VH, a CH1 domain, optionally a first hinge region, a first Fc region (e.g., CH2 domain and CH3 domain); (b) a second polypeptide including, preferably from N-terminus to C-terminus: a first VHH, optionally a second hinge region, a second Fc region (e.g., CH2 domain and CH3 domain); and (c) a third polypeptide including, preferably from N-terminus to C-terminus: a second VHH, a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 11. VHH-Knob+VHH-Fab-Hole (404, 410, 510, 610, 710, 804)

As shown in FIGS. 8D (404), 8J (410), 11D (510), 11J (610), 11P (710), 15D (804), a multispecific antibody (e.g., trispecific antibody) can be prepared, which includes (a) a first polypeptide including, preferably from N-terminus to C-terminus: a first VHH, optionally a first hinge region, a first Fc region (e.g., CH2 domain and CH3 domain); (b) a second polypeptide including, preferably from N-terminus to C-terminus: a VH, a CH1 domain, optionally a second hinge region, a second Fc region (e.g., CH2 domain and CH3 domain); and (c) a third polypeptide including, preferably from N-terminus to C-terminus: a second VHH, a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 12. VHH-Fab-Knob+VHH-Hole (405, 411, 511, 611, 711, 805)

As shown in FIGS. 8E (405), 8K (411), 11E (511), 11K (611), 11Q (711), and 15E (805), a multispecific antibody (e.g., trispecific antibody) can be prepared, which includes (a) a first polypeptide including, preferably from N-terminus to C-terminus: a first VHH, a VH, a CH1 domain, optionally a first hinge region, a first Fe region (e.g., CH2 domain and CH3 domain); (b) a second polypeptide including, preferably from N-terminus to C-terminus: a second VHH, optionally a second hinge region, a second Fe region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fe region comprises one or more knob mutations. In some embodiments, the second Fe region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 13. VHH-Knob+VHH-Fab-Hole (406, 412, 512, 612, 712, 806)

As shown in FIGS. 8F (406), 8L (412), 11F (512), 11L (612), 11R (712), and 15F (806), a multispecific antibody (e.g., trispecific antibody) can be prepared, which includes (a) a first polypeptide including, preferably from N-terminus to C-terminus: a first VHH, optionally a first hinge region, a first Fc region (e.g., CH2 domain and CH3 domain); (b) a second polypeptide including, preferably from N-terminus to C-terminus: a second VHH, a VH, a CH1 domain, optionally a second hinge region, a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fe region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 14. Fab-Knob+VHH—VHH-Hole (413, 415, 813, 815)

As shown in FIGS. 8M (413), 8O (415), 15G (813), and 15L (815), a multispecific antibody (e.g., trispecific antibody) can be prepared, which includes (a) a first polypeptide including, preferably from N-terminus to C-terminus: a VH, a CH1 domain, optionally a first hinge region, a first Fe region (e.g., CH2 domain and CH3 domain); (b) a second polypeptide including, preferably from N-terminus to C-terminus: a first VHH, a second VHH, optionally a second hinge region, a second Fe region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### 15. VHH—VHH-Knob+Fab-Hole (414, 416, 814)

As shown in FIGS. 8N (414), 8P (416), and 15K (814), a multispecific antibody (e.g., trispecific antibody) can be prepared, which includes (a) a first polypeptide including, preferably from N-terminus to C-terminus: a first VHH, a second VHH, optionally a first hinge region, a first Fc region (e.g., CH2 domain and CH3 domain); (b) a second polypeptide including, preferably from N-terminus to C-terminus: a VH, a CH1 domain, optionally a second hinge region, a second Fc region (e.g., CH2 domain and CH3 domain); and (c) a third polypeptide including, preferably from N-terminus to C-terminus: a VL, and a CL. In some embodiments, the VH and the VL associate with each other, forming an antigen binding site of a Fab that specifically binds to a T cell antigen. In some embodiments, the VHH specifically binds to a tumor-associated antigen.

In some embodiments, the first Fc region comprises one or more knob mutations. In some embodiments, the second Fc region comprises one or more hole mutations. In some embodiments, the first polypeptide comprises a sequence that is at least 80%, 85%, 90%, 95%, 96%, 97%, 98%, 99%, or 100% identical to SEQ ID NO: 1. In some embodiments, the second polypeptide comprises a sequence that is at least 80%, 85%, 90%, 95%, 96%, 97%, 98%, 99%, or 100% identical to SEQ ID NO:2.

In some embodiments, the Fab can target CD3 (e.g., human CD3). In some embodiments, the VH comprises a sequence that is at least 80%, 90%, 95%, or 100% identical to SEQ ID NO: 5 and the VL comprises a sequence that is at least 80%, 90%, 95%, or 100% identical to SEQ ID NOs: 6. In some embodiments, the VHH can target CEA, and comprises a sequence that is at least 80%, 85%, 90%, 95%, 96%, 97%, 98%, 99%, or 100% identical to SEQ ID NO: 7. In some embodiments, the VHH can target EGFR, and comprises a sequence that is at least 80%, 85%, 90%, 95%, 96%, 97%, 98%, 99%, or 100% identical to SEQ ID NO: 8. In some embodiments, the VHH can target HER2, and comprises a sequence that is at least 80%, 85%, 90%, 95%, 96%, 97%, 98%, 99%, or 100% identical to SEQ ID NO: 9. In some embodiments, the VHH can target CEA-CAM6, and comprises a sequence that is at least 80%, 85%, 90%, 95%, 96%, 97%, 98%, 99%, or 100% identical to SEQ ID NO: 10. In some embodiments, the first hinge region and/or the second hinge region are derived from the hinge region of human IgG4.

### Antigen-Binding Protein Construct Characteristics

The anti-CD3, anti-TAA (tumor associated antigen), or anti-CD3/TAA antigen-binding protein construct (e.g., antibodies, bispecific antibodies, trispecific antibodies, multispecific antibodies, or antibody fragments thereof), can include an antigen binding site that is derived from any anti-CD3 antibody, anti-TAA antibody (e.g., anti-CEA, anti-CEA-CAM6, anti-EGFR or anti-HER2), or any antigen-binding fragment thereof as described herein.

In some embodiments, the antibodies or antigen-binding fragments thereof described herein are CEA antagonist. In some embodiments, the antibodies or antigen-binding fragments thereof are CEA agonist. In some embodiments, the antibodies or antigen-binding fragments thereof described herein are CEA-CAM6 antagonist. In some embodiments, the antibodies or antigen-binding fragments thereof are CEA-CAM6 agonist. In some embodiments, the antibodies or antigen-binding fragments thereof as described herein are CD3 antagonist. In some embodiments, the antibodies or antigen-binding fragments thereof are CD3 agonist. In some embodiments, the antibodies or antigen-binding fragments thereof as described herein are EGFR antagonist. In some embodiments, the antibodies or antigen-binding fragments thereof are EGFR agonist. In some embodiments, the antibodies or antigen-binding fragments thereof as described herein are HER2 antagonist. In some embodiments, the antibodies or antigen-binding fragments thereof are HER2 agonist.

In some embodiments, the antibodies, or antigen-binding fragments thereof described herein can bind to CD3 and a target tumor associated antigen (e.g., CEA, CEA-CAM6, EGFR or HER2), thereby bridging T cells and target cells; activating T cells; and inducing directly killing the cancer cells by the T cells.

In some embodiments, the antibody (or antigen-binding fragments thereof) specifically binds to the target antigen (e.g., CD3, CEA, CEA-CAM6, EGFR or HER2) with a dissociation rate (koff, kdis) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon, ka) is greater than 1×10² M⁻¹s⁻¹, greater than 1×10³ M⁻¹s⁻¹, greater than 1×10⁴ M⁻¹s⁻¹, greater than 1×10⁵ M⁻¹s⁻¹, greater than 1×10⁶ M⁻¹s⁻¹. In some embodiments, kinetic association rates (kon) is less than 1×10⁵ M⁻¹s⁻¹, less than 1×10⁶ M⁻¹s⁻¹, or less than 1×10⁷ M⁻¹s⁻¹.

Affinities can be deduced from the quotient of the kinetic rate constants (KD=koff/kon). In some embodiments, KD is less than 1×10⁻⁴M, less than 1×10⁻⁵M, less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, KD is greater than 1×10⁻⁴ M, greater than 1×10⁻⁵ M, greater than 1×10⁻⁶ M, greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. Furthermore, Ka can be deduced from KD by the formula Ka=1/KD.

General techniques for measuring the affinity of an antibody for an antigen include, e.g., ELISA, RIA, and surface plasmon resonance (SPR).

In some embodiments, the expression level of the antibodies, the antigen binding fragments thereof, or the antigen-binding protein constructs described herein is at least or about 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 50, 100, 200, 300, 400, 500, 600, 700, 800, 900, 1000, 2000, 3000, 4000, 5000, 6000, 7000, 8000, 9000, 10000, 20000, 50000, or 100000 mg/L, as determined using the method described herein. In some embodiments, the percentage of multispecific antibody (e.g., bispecific antibody or trispecific antibody) formed, as determined by size-exclusion chromatography as described herein, is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 93%, at least 94%, at least 95%, at least 96%, or at least 97% of the total protein level.

In some embodiments, the antibodies, the antigen binding fragments thereof, or the antigen-binding protein constructs described herein have a T cell mediated cell killing EC50 of less than 0.1 nM, less than 0.2 nM, less than 0.5 nM, less than 0.8 nM, less than 1 nM, less than 2 nM, less than 5 nM, less than 8 nM, less than 10 nM, less than 20 nM, less than 50 nM, less than 100 nM, less than 200 nM, less than 500 nM, or less than 800 nM, as determined using the method described herein. In some embodiments, the antibodies, the antigen binding fragments thereof, or the antigen-binding protein constructs described herein have a T cell mediated cell killing EC50 of more than 0.1 nM, more than 0.2 nM, more than 0.5 nM, more than 0.8 nM, more than 1 nM, more than 2 nM, more than 5 nM, more than 8 nM, more than 10 nM, more than 20 nM, more than 50 nM, more than 100 nM, more than 200 nM, more than 500 nM, or more than 800 nM, as determined using the method described herein. In some embodiments, the antibodies, antigen binding fragments thereof, or the antigen-binding protein constructs described herein have a T cell mediated cell killing EC50 value that is 0.1-1 nM, 0.2-2 nM, 0.5-5 nM, 0.8-8 nM, 1-10 nM, 2-20 nM, 5-50 nM, 8-80 nM, 10-100 nM, 20-200 nM, 50-500 nM, or 80-800 nM. In some embodiments, the antibodies, the antigen binding fragments thereof, or the antigen-binding protein constructs described herein have a T cell mediated cell killing EC50 value that is less than or about 50%, less than or about 30%, less than or about 20%, less than or about 10%, less than or about 5%, less than or about 1% as compared to that of an isotype control antibody.

In some embodiments, the antibodies, the antigen binding fragments thereof, the antigen-binding protein constructs, or protein complexes described herein have a cell-binding EC50 of less than or about 0.1 nm, 0.25 nM, 0.5 nM, 0.75 nM, 1 nM, 1.25 nM, 1.5 nM, 2 nM, 2.5 nM, 5 nM, 7.5 nM, 10 nM, or 20 nM, as determined using the methods described herein.

In some embodiments, the antibodies, the antigen binding fragments thereof, the antigen-binding protein constructs, or protein complexes described herein have a melting temperature (Tm) value of higher than 60° C., 60.5° C., 61° C., 61.5° C., 62° C., 62.5° C., 63° C., 63.5° C., 64° C., 65° C., 66° C., 67° C., 68° C., 69° C., 70° C., 71° C., 72° C., 73° C. or 74° C., as determined using the methods described herein.

In some embodiments, the antibodies, the antigen binding fragments thereof, the antigen-binding protein constructs, or protein complex described herein have a tumor growth inhibition percentage (TGI_{TV}%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI_{TV}% can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI_{TV}%) is calculated using the following formula: TGI_{TV}(%)=[1−(Ti−T0)/(Vi−V0)]×100 (Ti is the average tumor volume in the treatment group on day i. T0 is the average tumor volume in the treatment group on day zero. Vi is the average tumor volume in the control group on day i. V0 is the average tumor volume in the control group on day zero).

In some embodiments, the antibodies, the antigen binding fragments thereof, the antigen-binding protein constructs, or protein complex described herein have a serum stability of more than 50%, 55%, 60%, 65% 70%, 75%, 80%, 85%, 90%, 91%, 92%, 93%, 94%, 95%, 96%, 97%, 98% or 99% cell binding after 1-10 days of storage in human serum, as determined using the methods described herein.

Thermal stabilities can also be determined. The antibodies, the antigen-binding fragments thereof, or the antigen-binding protein constructs (e.g., bispecific antibody) as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows two transitions, with a first denaturation temperature, Tm1, and a second denaturation temperature Tm2. The presence of these two peaks often indicate the denaturation of the Fc domains (Tm1) and Fab domains (Tm2), respectively. When there are two peaks, Tm usually refers to Tm2. Thus, in some embodiments, the antibodies or antigen binding fragments as described herein has a Tm1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 T. In some embodiments, the antibodies or antigen binding fragments as described herein has a Tm2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 TG. In some embodiments, Tm, Tm1, Tm2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies, the antigen binding fragments thereof, or the antigen-binding protein constructs have a self-interaction signal in the range of between 0.037 nm and 0.121 nm. In some embodiments, the antibodies, the antigen binding fragments thereof, or the antigen-binding protein constructs have a self-interaction signal that is less than 0.01 nm, less than 0.02 nm, less than 0.03 nm, less than 0.04 nm, less than 0.05 nm, less than 0.06 nm, less than 0.07 nm, less than 0.08 nm, less than 0.09 nm, less than 0.1 nm, less than 0.11 nm, less than 0.12 nm, or less than 0.13 nm. In some embodiments, the antibodies, the antigen binding fragments thereof, or the antigen-binding protein constructs have a self-interaction signal that is more than 0.01 nm, more than 0.02 nm, more than 0.03 nm, more than 0.04 nm, more than 0.05 nm, more than 0.06 nm, more than 0.07 nm, more than 0.08 nm, more than 0.09 nm, more than 0.1 nm, more than 0.11 nm, more than 0.12 nm, or more than 0.13 nm. In some embodiments, the antibodies, the antigen binding fragments thereof, or the antigen-binding protein constructs have a self-interaction signal that is 0.01-0.05 nm, 0.02-0.06 nm, 0.03-0.07 nm, 0.04-0.08 nm, 0.05-0.09 nm, 0.06-0.1 nm, 0.07-1.1 nm, 0.08-1.2 nm, or 0.09-1.3 nm.

In some embodiments, the antibodies, the antigen binding fragments thereof, or the antigen-binding protein constructs have a functional Fc region. In some embodiments, effector function of a functional Fc region is antibody-dependent cell-mediated cytotoxicity (ADCC) and/or complement-dependent cytotoxicity (CDC). In some embodiments, the Fc region is human IgG1, human IgG2, human IgG3, or human IgG4.

In some embodiments, the antibodies, the antigen binding fragments thereof, or the antigen-binding protein constructs do not have a functional Fc region. For example, the antibodies or antigen binding fragments are Fab, Fab′, F(ab′)₂, and Fv fragments. In some embodiments, the antibodies, antigen binding fragments, or the antigen-binding protein constructs have a Fc region that includes one or more mutations to reduce the effector function. In some embodiments, the antibodies, the antigen binding fragments thereof, or the antigen-binding protein constructs described herein do not have antibody-dependent cell-mediated cytotoxicity (ADCC) or complement dependent cytotoxicity (CDC).

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the multi-specific antibody including the trispecific antibody described herein (e.g., CD3/CEA-CAM6/EGFR trispecific antibody) has an asymmetric structure comprising: 2, 3, 4, 5, or 6 antigen binding sites. In some embodiments, the multispecific antibody described herein comprises 2, 3, 4, 5, or 6 antigen binding sites (e.g., antigen binding scFv domains, Fab, or VHH) that target CEA-CAM6. In some embodiments, the CEA-CAM6 binding Fab domain comprises the same variable domain sequence. In some embodiments, the CEA-CAM6 binding Fab domain comprises different variable domain sequences.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68 (1-3):9-16 (October 1997); Morea et al., J Mol Biol. 275 (2):269-94 (January 1998); Chothia et al., Nature 342 (6252):877-83 (December 1989); Ponomarenko and Bourne, BMC Structural Biology 7:64 (2007); Kontermann, R., & Dübel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody or antigen-binding protein can include an intact immunoglobulin molecule (e.g., IgG1, IgG2a, IgG2b, IgG3, IgM, IgD, IgE, IgA) or fragments thereof. The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody or antigen-binding protein can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen-binding domain” or “antigen-binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a trispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antibodies or antigen-binding fragments thereof can bind to two different antigens or two different epitopes. In some embodiments, the antibodies or antigen-binding fragments thereof can bind to three different antigens or three different epitopes.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

In some embodiments, the Fc region can be further modified to increase or decrease effector functions as well as serum half-life.

Any of the antibodies, antigen-binding fragments thereof, or antigen-binding proteins described herein can be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of the antibodies, antigen-binding fragments thereof, or antigen-binding proteins in vitro (e.g., in tissue culture or when stored as a pharmaceutical composition) or in vivo (e.g., in a human).

In some embodiments, the antibodies, antigen-binding fragments thereof, or antigen-binding proteins (e.g., multispecific antibodies) described herein can be conjugated to a therapeutic agent. The antibody-drug conjugate comprising the antibodies, antigen-binding fragments thereof, or antigen-binding proteins can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multispecific antibody or antigen-binding fragment thereof described herein (e.g., CD3/CEA-CAM6/EGFR multispecific antibody) binds to CD3 (e.g., human CD3) with a binding affinity that is about 50%, about 60%, about 70%, about 80%, about 90%, about 100%, about 110%, about 120%, about 130%, about 140%, about 150%, or about 200% to that of an antibody (e.g., an anti-CD3 antibody) comprising the same antigen binding region (e.g., Fab, scFv or VHH) of the multi-specific antibody.

In some embodiments, the multispecific antibody or antigen-binding fragment thereof described herein (e.g., CD3/CEA-CAM6/EGFR multispecific antibody) binds to CEA-CAM6 with a binding affinity that is about 50%, about 60%, about 70%, about 80%, about 90%, about 100%, about 110%, about 120%, about 130%, about 140%, about 150%, or about 200% to that of an antibody (e.g., an anti-CEA-CAM6 antibody) comprising the same antigen binding region (e.g., Fab, scFv or VHH) of the multi-specific antibody.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof or the antigen-binding protein constructs by recombinant techniques.

As used herein, a “vector” is any construct capable of delivering one or more polynucleotide (s) of interest to a host cell when the vector is introduced to the host cell. An “expression vector” is capable of delivering and expressing the one or more polynucleotide (s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HEK293 cells; and plant cells. Appropriate culture mediums and conditions for the host cells described herein are known in the art.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antibodies or Antigen-Binding Protein Constructs

An isolated fragment of human protein (e.g., CD3 or CEA-CAM6) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding affinities.

Variants of the antibodies, antigen-binding fragments, or the antigen-binding protein constructs described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen (s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fe region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. In some embodiments, the methods described here are designed to make a trispecific antibody. Bispecific or trispecific antibodies can be made by engineering the interface between different antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain (s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions T366W. The other heavy chain can have one or more the following substitutions T366S, L368A, and Y407V. Furthermore, a substitution (-ppcpScp-->-ppcpPcp-) can also be introduced at the hinge regions of both substituted IgG.

Furthermore, an anion-exchange chromatography can be used to purify the antibodies or antigen binding fragments described herein. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific or trispecific antibody having Arm A and Arm B will be somewhere between the two pIs of the homodimers. Thus, the two homodimers and the bispecific antibody or trispecific antibody can be released at different pH conditions. The present disclosure shows that a few amino acid residue substitutions can be introduced to the heavy chains to adjust pI.

### Methods of Treatment

The methods described herein include methods for the treatment of disorders associated with cancer. Generally, the methods include administering a therapeutically effective amount of engineered multispecific antibodies (e.g., bispecific antibodies or trispecific antibodies) or the antigen-binding protein constructs as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., antigen-binding protein constructs) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, antigen-binding protein constructs, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing CEA-CAM6.

In some embodiments, the cancers are lung cancers, colorectal cancer, head and neck cancer, stomach cancer, pancreatic cancer, urothelial cancer, breast cancer, cervical cancer, or endometrial cancer.

In some embodiments, the cancer cells described herein is cell lines, e.g., H1395 cells.

In some embodiments, the cancer cells have an elevated CEA-CAM6 level, e.g., at least 10%, at least 20%, at least 30%, at least 40%, at least 50% higher than non-cancerous cells.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antigen-binding protein constructs, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, an antigen-binding protein construct, or an antibody-drug conjugate is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the agent used.

Effective amounts and schedules for administering the antibodies, antigen-binding protein constructs, antibody-encoding polynucleotides, antibody-drug conjugates, and/or compositions disclosed herein may be determined empirically, and making such determinations is within the skill in the art.

A typical dosage of an effective amount of an antibody or antigen-binding protein construct is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antigen-binding protein constructs, antibody-drug conjugates, or pharmaceutical composition (e.g., any of the antibodies, antigen-binding fragments, antigen-binding protein constructs, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFN-α, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody, antigen-binding fragment thereof, or the antigen-binding protein construct can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antigen-binding protein constructs, antigen binding proteins, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies, antigen-binding fragments thereof, or antigen-binding protein constructs (e.g., any of the antibodies, antibody fragments, or antigen-binding protein constructs described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies, antigen-binding fragments, or antigen-binding protein constructs described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, antigen-binding protein constructs, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody, antibody fragment, or antigen-binding protein constructs in vivo.

The pharmaceutical compositions can be included in a container, pack, or dispenser together with instructions for administration. The disclosure also provides methods of manufacturing the antibodies, antigen binding fragments thereof, or antigen-binding protein constructs for various uses as described herein.

### EXAMPLES

The invention is further described in the following examples, which do not limit the scope of the invention described in the claims.

### Materials and Methods

### Preparation of Human Peripheral Blood Mononuclear Cells (PBMC)

PBMCs were prepared by density gradient centrifugation, and blood samples were from blood banks or healthy human donors.

The blood of healthy donors was stored in an EDTA-containing anticoagulation tube, and allowed to stand for 10 minutes. An equal volume of 2% PBS (pH 7.4) was added and mix thoroughly. 15 ml Ficoll Paque PLUS low-density centrifugal fluid (GE) was added into a 50 ml Falcon tube, and 30 ml of diluted fresh blood was aspirated and slowly added to the upper layer of the density gradient solution along the side wall of the Falcon tube. The centrifuge was adjusted to the brake off state, the test tube was loaded on the centrifuge, and centrifuged at 1450 rpm at room temperature for 45 minutes. The test tube was gently taken out and visually observed to make sure that the blood in the centrifuge tube was divided into three layers (the upper layer is the serum layer, the white part of the middle layer is the peripheral blood mononuclear cells, and the bottom layer is the red blood cells). The upper yellow and transparent serum layer was carefully aspirated, leaving only the middle layer, which was transferred to a new 15 ml centrifuge tube. An equal volume of the gradient solution was added and the diluted middle layer was centrifuged at 1450 rpm for 30 minutes at room temperature. The upper suspension was removed, retaining the bottom cell pellet. The cells were re-suspended and washed 3 times until the upper layer was clear. 1 ml of 2% FBS-PBS buffer was added to re-suspend the cells. After the PBMC was counted, the cells were re-suspended in the RPMI1640 medium containing 10% FBS (GIBCO) and 1% L-glutamine (GIBCO) and cultured in a CO₂ incubator at 37° C. and 5% CO₂.

### DNA Sequencing and Synthesis

The DNA sequence was determined by double-stranded DNA sequencing, and the sequencing was performed by GENEWIZ (Suzhou Jinweizhi Biotechnology Co., Ltd.).

All genes involved in these experiments were synthesized by GenScript (Nanjing GenScript Biotechnology Co., Ltd.).

### Example 1. Construction of CD3/CEA/EGFR Trispecific Antibodies

On the basis of mouse CD3 antibody, the humanized CD3 antibody TA1 was obtained by mutation, library construction, humanization transformation and multiple screening.

CEA and EGFR single-domain antibodies (VHH) were obtained by immunizing llamas (lama). 250 μg of recombinant human tumor antigen CEA (CEACAM-5/CD66e, ACRO systems, CE5-H5226) or human EGFR protein (ACRO systems, EGR-H5222) was used to subcutaneously immunize adult alpacas, once every other month for 5 times in total. PBMC cells were isolated, RNA was extracted, and phage antibody library was constructed. After the library was packaged to form phage particles, the liquid phase method was used for panning. The phage was combined with biotinylated CEA antigen liquid or EGFR antigen liquid, and then separated by streptavidin-labeled magnetic beads. The phage was screened using multiple methods (such as ELISA and engineered cells overexpressing CEA or EGFR), to obtain CEA and EGFR single-domain antibodies, which are referred to as CEA and EGFR.

Non-unilateral CD3/CEA/EGFR tertiary antibody structure design: 12 tertiary antibody structures (TA1-Fab CD3 monovalent, CEA monovalent and EGFR monovalent) were designed according to FIG. 2. TA1-Fab is located at the N-terminus of Fc, and the three antigen-binding domains (TA1-Fab, EGFR single-domain antibody and CEA single-domain antibody) are not located at the N-terminus or C-terminus of Fe at the same time. When the single-domain antibody VHH is located at the C-terminus of Fc, GGGGSGGGGS (SEQ ID NO: 11) acts as a linker to connect the two fragments. When the single-domain antibody VHH is located at the N-terminus of Fc, the Fc hinge region (Hinge) (SEQ ID NO: 12) acts as a linker to connect the two fragments. At the same time, bispecific control antibodies containing TA1-Fab for CEA or EGFR (CEA-BsAb and EGFR-BsAb) were designed based on FIGS. 1A-1D. “TA1F” represents the Fab region of the anti-CD3 antibody TAL. TA1 VH is linked to CH1-Hinge-Fc (IgG4), and TA1 VL is linked to CL (kappa).

In some embodiments, the structures of the trispecific antibodies are shown in FIGS. 2A-2L. The asymmetric structure design of two chains containing Fc is adopted, and there are 1 CEA antigen-binding domain, 1 EGFR antigen-binding domain and 1 CD3 antigen-binding domain. CEA and EGFR are in the form of single-domain antibodies (VHH), CD3 is in the form of a Fab. The Fc region endows the antibody with a long half-life and good stability. At the same time, the Knob into Hole (KIH) design of the two heavy chains greatly reduces the chance of mismatch and improves the yield and homogeneity. The structures (Format) of the specific bispecific and trispecific antibodies are shown in FIGS. 1A-1D and FIGS. 2A-2L, respectively.

The two heavy chain IgG4 Fes are knob-Fc and hole-Fc, respectively. The sequences of knob-Fc and hole-Fc are preferably those shown in the below table. The light chain is a common kappa light chain, and CH1 is derived from IgG4.

TABLE 1
Common Sequences

| Name | SEQ ID NO. | Sequence |
|---|---|---|
| knob-Fc | 1 | ESKYGPPCPPCPAPEAAGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTISKAKG |
| | | QPREPQVYTLPPCQEEMTKNQVSLWCLVKGFYPSDIAVEWESNGQPENNYKTTPPVLDSDG |
| | | SFFLYSRLTVDKSRWQEGNVFSCSVMHEALHNHYTQKSLSLSL |
| hole-Fc | 2 | ESKYGPPCPPCPAPEAAGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTISKAKG |
| | | QPREPQVCTLPPSQEEMTKNQVSLSCAVKGFYPSDIAVEWESNGQPENNYKTTPPVLDSDG |
| | | SFFLVSRLTVDKSRWQEGNVFSCSVMHEALHNRFTQKSLSLSL |
| CL | 3 | RTVAAPSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| CH1(IgG4) | 4 | ASTKGPSVFPLAPCSRSTSESTAALGCLVKDYFPEPVTVSWNSGALTSGVHTFPAVLQSSGL |
| | | YSLSSVVTVPSSSLGTKTYTCNVDHKPSNTKVDKRV |

The variable region sequence of the CD3 antibody TA1, the heavy chain sequence of the CEA and EGFR single domain antibodies are shown in the below table.

TABLE 2
Sequence Listing of CD3 Variable Region, CEA and EGFR Single Domain
Antibodies

| Name | SEQ ID NO. | Sequence |
|---|---|---|
| TA1 VH | 5 | EVKLLESGGGLVQPKGSLKLSCAASGFTFNTYAMNWVRQAPGKGLEWVARIRSKYNN |
| | | YATYYADSVKDRFTISRDDSQSILYLQMNNLKTEDTAMYYCVRHGNFGNSYVSWFAY |
| | | WGQGTLVTVSS |
| TA1 VL | 6 | QAVVTQESALTTSPGETVTLTCRSSTGAVTTSNYANWVQEKPDHLFTGLIGGTNKRAPG |
| | | VPARFSGSLIGDKAALTITGAQTEDEAIYFCALWYSNLWVFGGGTKVEIK |
| CEA(VHH) | 7 | EVQLVESGGGFVQAGESLTLSCTSSTLTFTPYRMAWYRQAPGKQRDLVADISSGDGRTT |
| | | NYADFAKGRFTISRDNIKNTVFLRMTNLKPEDTAVYYCNTFVSFVGIARSWGQGTQVTV |
| | | SS |
| EGFR(VHH) | 8 | QVQLQESGGGSVQAGGSLKLSCAASGRGFSRYAMGWFRQAPGQDREFVATISWTNSTD |
| | | YADSVKGRFAISRDNAKNTAYLQMNSLKPEDTAVYYCAADKWASSTRSIDYDYWGQG |
| | | IQVTVSS |

Construction of plasmid: all genes involved in this application were synthesized by GenScript (Nanjing GenScript Biotechnology Co., Ltd.), and then connected to mammalian expression vector pEE12.4 by enzyme digestion. The variable regions of the heavy and light chain DNA sequences were cloned into pEE12.4 in frame with the pre-inserted human IgG4 heavy chain constant regions or kappa light chain constant regions. The plasmids were extracted with OMEGA's Plasmid Extraction Kit and stored at −80° C.

### Example 2: Preparation of Non-Unilateral CD3/CEA/EGFR Trispecific Antibodies

Expi 293F™ cells (ThermoFisher) were transfected with the above plasmids with the aid of polyethyleneimine (PEI, POLYETHYLENEIMINE ‘MAX; polysciences, 24765-2). Cells were transfected with corresponding expression plasmids in a 1:2:1 ratio [“heavy chain Fc(hole)”: “light chain”: “heavy chain Fc(knob)” ].

Expi 293F™ cells were cultured in suspension in CD OptiCHO™ medium at 37° C. (5% CO₂, 135 rpm). One day before transfection, 293F cells were passaged into a 1 L gas-permeable conical flask (Corning) at a seeding density of 1.0×10⁶ cells/mL and a cell volume of 200 mL. The estimated cell density on the day of transfection was 1.8-2.0×10⁶ cells/mL. The cell suspension was centrifuged at 1000 rpm for 5 min at room temperature, washed once with Expi293 medium. The cells were collected, re-suspended with 200 mL of Expi293 medium. 400 μg of plasmid was diluted with 5 mL of Opti-MEM medium, and vortexed for 15 s. 1.2 mg PEI was diluted with 5 mL Opti-MEM medium and vortexed for 15 s. The PEI-containing solution was added dropwise to the DNA-containing solution, mixed gently, and incubated at room temperature for 15 min. The plasmid/PEI mixture was added to the cell suspension, and incubated in a 37° C., 5% CO₂, 85 rpm incubator for 4 h. After 4 h, 200 mL EX-CELL™293 medium and 2 mM Glutamine (Gibco) were added, and the speed was adjusted to 135 rpm to continue the culture for 24 h. After 24 h, 3.8 mM VPA, a cell proliferation inhibitor, was added. After 72 h, 40 mL medium D was added. After 7 days, the cell suspension was centrifuged at 18,000 rpm for 30 min and supernatant was collected. The supernatant was sterile filtered with a 0.22 μm filter, and sodium azide was added to reach a final concentration of 0.01% w/v. The supernatant was stored at 4° C. for purification.

The target protein was purified using Protein A chromatography. The collected supernatant was loaded onto MabSelect Prism A FF (GE; 17-5498-01) equilibrated with 20 mL of equilibration buffer (25 mM Tris, 150 mM NaCl, pH 7.5). Unbound protein was removed by at least 10 column volumes of washing buffer (25 mM Tris, 150 mM NaCl, pH 7.5). The target protein was eluted with 5 column volumes of elution buffer (20 mM Na-Citrate, pH 3.5). The protein solution was neutralized by adding 1/10 volume of 1 M Tris, pH 9.5.

The target protein was transferred into the desired buffer using Zeba™ desalting spin columns (ThermoFisher) or ultrafiltration tubes (Millipore). Protein purity and concentration were determined by SDS-PAGE electrophoresis (loading: 2-3 μg) and NanoDrop2000.

The target protein was concentrated, filtered, and then loaded to a gel filtration column (Gel Filtration, HiLoad Superdex 200, GE) equilibrated with equilibration buffer (20 mM histidine, 140 mM sodium chloride, pH 6.0). At room temperature, the molecular weight, purity, aggregation of the sample were analyzed using analyzing buffer (PBS, pH 7.2) at a flow rate of 0.5 mL/min. The resulting high-purity monomeric protein is aliquoted and stored at −80° C.

In an alternative embodiment, proteins were purified from cell supernatants by protein A affinity chromatography (MabSelect SuRe, GE). The protein eluate was then subjected to cation exchange chromatography (HiTrap SP HP, GE) and subsequently analyzed using a gel filtration column. The target protein obtained using this purification method was >90% pure.

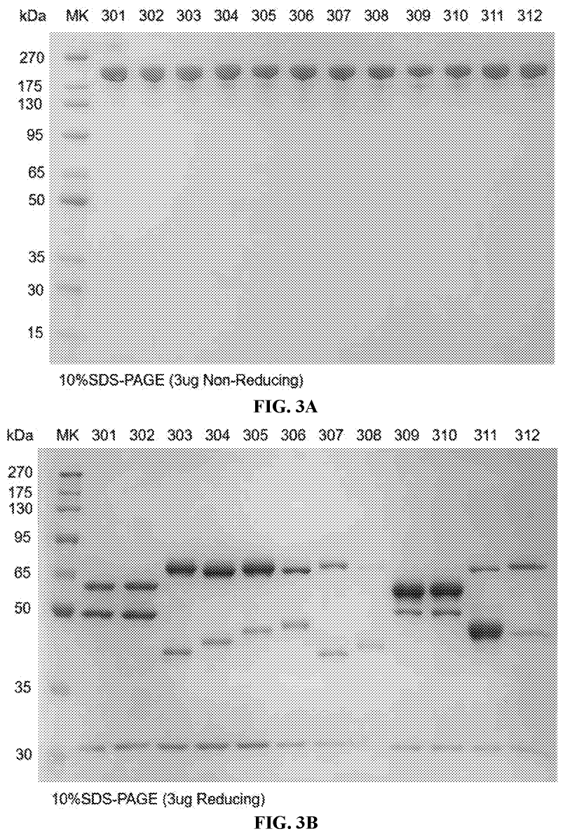

The protein structure design is shown in FIGS. 1A-1D and FIGS. 2A-2L, and the SDS-PAGE results are shown in FIGS. 3A-3B. The yield of the third antibody was 13.73-22.83 mg/L, and the purity was all >92%. See the below table for details.

TABLE 3
Yield and Purity of Non-Unilateral
CD3/CEA/EGFR Trispecific Antibody

| Sample | Yield (mg/L) | Purity % |
|---|---|---|
| 301 | 20.28 | 96.82 |
| 302 | 15.65 | 95.89 |
| 303 | 19.86 | 94.21 |
| 304 | 13.73 | 93.22 |
| 305 | 17.42 | 94.25 |
| 306 | 15.60 | 95.21 |
| 307 | 18.46 | 93.33 |
| 308 | 16.90 | 92.53 |
| 309 | 22.83 | 95.82 |
| 310 | 17.16 | 92.63 |
| 311 | 21.58 | 92.45 |
| 312 | 18.32 | 95.23 |

### Example 3: Determining the Affinity of Non-Unilateral CD3/CEA/EGFR Trispecific Antibody by Bio-Layer Interferometry (BLI)

Biofilm interferometry (BLI) experiments were conducted on OCTECT RED96e (ForteBio) at 30° C. with 0.02% PBST (10 mmol/L Na₂HPO₄; 1.75 mmol/L KH₂PO₄; 137 mmol/L NaCl; 2.65 mmol/L KCl; pH 7.2-7.4, 0.02% surfactant Tween 20) as the running buffer.

Antibodies were first captured onto an anti-human IgG Fc AHC biosensor (Anti-hIgG Fe, Sartorius, 18-5060). Antibodies then were diluted to 5 μg/mL and 1.5 nM protein was coupled to the AHC sensor surface in 0.02% PBST.

The analytes recombinant human CD3ε/δ protein (ACRO systems, CDD-H52W1), recombinant human tumor antigen CEA (CEACAM-5/CD66e, ACRO systems, CE5-H5226) and recombinant human EGFR (ACRO systems, CE5-H5226) were diluted separately systems, EGR-H5222) were diluted in 0.02% PBST to 200 nM, 100 nM, 50 nM, 25 nM, 12.5 nM, 6.25 nM, and 3.125 nM.

After the sensor captured the antibodies, it was allowed to bind and dissociate with different concentrations of analytes to determine the KD value of the interaction. The experimental parameters are as follows: Baseline1: 60 s, Loading: 240 s, Baseline2: 180 s, Association: 240 s, Dissociation: 600 s, High sensitivity kinetics: 2 Hz. Kinetic constants were derived based on the rate equation for 1:1 Langmuir binding to obtain equilibrium dissociation constants (KD). The Data Analysis HT 12 software (Sartorius, 50-5029) was used to simultaneously fit binding and dissociation curves to calculate Kₒₙ (ka) and K_{off} (kdis). The affinity KD was calculated based on the below formula: KD=K_{off}/Kₒₙ.

The experimental results showed that the affinity of the non-unilateral trispecific antibody to CEA was 0.4 nM-1.2 nM, the affinity to EGFR was 5.8 nM-26.7 nM, and the affinity to CD3 was 0.5 nM-5.9 nM. The fitted affinity KD data are shown in the tables below.

TABLE 4
Affinity of non-unilateral CD3/CEA/EGFR trispecific
antibody to CEA (T = 30° C.)
Antigen: CEA

| Sample | ka (1 M⁻¹s⁻¹) | kdis (s−1) | KD (M) |
|---|---|---|---|
| 301 | 4.44E+05 | 2.46E−04 | 5.54E−10 |
| 302 | 3.38E+05 | 2.71E−04 | 8.00E−10 |
| 303 | 5.33E+05 | 2.35E−04 | 4.41E−10 |
| 304 | 2.61E+05 | 2.31E−04 | 8.85E−10 |
| 305 | 2.35E+05 | 2.79E−04 | 1.19E−09 |
| 306 | 2.74E+05 | 2.99E−04 | 1.09E−09 |
| 307 | 3.84E+05 | 2.01E−04 | 5.23E−10 |
| 308 | 2.61E+05 | 2.29E−04 | 8.76E−10 |
| 309 | 5.03E+05 | 2.24E−04 | 4.46E−10 |
| 310 | 2.81E+05 | 2.40E−04 | 8.54E−10 |
| 311 | 3.60E+05 | 3.73E−04 | 1.04E−09 |
| 312 | 2.25E+05 | 2.14E−04 | 9.50E−10 |
| CEA-BsAb | 5.45E+05 | 2.18E−04 | 3.99E−10 |

TABLE 5
Affinity of non-unilateral CD3/CEA/EGFR trispecific
antibody to EGFR (T = 30° C.)
Antigen: EGFR

| Sample | ka (1 M⁻¹s⁻¹) | kdis (1/s) | KD (M) |
|---|---|---|---|
| 301 | 4.40E+05 | 5.89E−03 | 1.34E−08 |
| 302 | 6.91E+05 | 4.18E−03 | 6.05E−09 |
| 303 | 4.97E+05 | 5.15E−03 | 1.04E−08 |
| 304 | 6.79E+05 | 3.95E−03 | 5.82E−09 |
| 305 | 4.02E+05 | 5.48E−03 | 1.36E−08 |
| 306 | 3.89E+05 | 5.48E−03 | 1.41E−08 |
| 307 | 3.57E+05 | 5.61E−03 | 1.57E−08 |
| 308 | 4.82E+05 | 3.78E−03 | 7.85E−09 |
| 309 | 3.42E+05 | 5.68E−03 | 1.66E−08 |
| 310 | 4.45E+05 | 3.90E−03 | 8.76E−09 |
| 311 | 2.22E+05 | 5.94E−03 | 2.67E−08 |
| 312 | 2.52E+05 | 6.28E−03 | 2.49E−08 |

TABLE 6
Affinity of non-unilateral CD3/CEA/EGFR trispecific
antibody to CD3ε/δ (T = 30° C.)
Antigen: CD3ε/δ

| Sample | ka (1 M⁻¹s⁻¹) | kdis (1/s) | KD (M) |
|---|---|---|---|
| 301 | 1.53E+05 | 7.32E−04 | 4.77E−09 |
| 302 | 1.28E+05 | 7.32E−04 | 5.70E−09 |
| 303 | 1.31E+05 | 7.74E−04 | 5.89E−09 |
| 304 | 1.20E+05 | 6.56E−04 | 5.49E−09 |
| 305 | 1.30E+05 | 7.09E−04 | 5.47E−09 |
| 306 | 1.31E+05 | 6.20E−04 | 4.74E−09 |
| 307 | 1.43E+05 | 5.77E−04 | 4.05E−09 |
| 308 | 1.78E+05 | 9.05E−04 | 5.09E−09 |
| 309 | 1.48E+05 | 2.70E−04 | 1.83E−09 |
| 310 | 2.47E+05 | 6.15E−04 | 2.49E−09 |
| 311 | 2.87E+05 | 1.44E−04 | 5.00E−10 |
| 312 | 9.22E+04 | 2.59E−04 | 2.81E−09 |

### Example 4: Non-Unilateral CD3/CEA/EGFR Trispecific Antibodies Induce T Cell Mediated Killing of EGFR-Positive Tumor Target Cells

T cells mediated killing induced by trispecific antibodies against target cells were assessed in human tumor cells NCI-H1299 (CEA negative; EGFR medium), NCI-H157 (CEA negative; EGFR medium) and NCI-H1395 (CEA medium; EGFR medium). Human PBMCs were used as effectors and killing was detected after 72 h incubation with antibodies.

Briefly, tumor cells were digested with trypsin/EDTA, washed once with pre-chilled PBS, re-suspended in RPMI 1640 medium containing 10% FBS, and plated at a density of 5,000 cells/well in a flat-bottom 96-well plate (Corning 3599). After culturing for 4 h, 50 μL of serially diluted antibody solution was added to each well. Each concentration was added to three replicate wells. The antibodies were incubated with the tumor cells for more than 30 min to fully attach the proteins to the cells. PBMC cells (Reid Bio, 1521) were thawed quickly, resuscitated, added to 10 mL of RPMI 1640 medium containing 10% FBS, centrifuged at 1000 rpm for 5 min, and re-suspended after discarding the supernatant. Cell density was adjusted and 50 μL of PBMCs was added to each well of target cells to reach a final E:T ratio of 10:1. The 96-well plate was placed in a 37° C., 5% CO₂ incubator for 3 days.

After culturing for 3 days, the cell culture supernatant was carefully removed. The plate was pat dried with a paper towel. Serum-Free DMEM containing 10% CCK8 detection solution (Dojindo, CK04-20) was added to the 96-well plate and incubated at 37° C. for 2-4 hours. After 4 hours, the absorbance at 450 nm was detected using a microplate reader (BioTech, Synergy LX). The cell viability was calculated according to the below formula:

cell viability=[(As−Ab)/(Ac−Ab)]×100%.
As: experimental well (medium containing cells, CCK-8, and test antibodies);
Ac: control wells (medium containing cells, CCK-8, without test antibodies);
Ab: blank well (medium without cells and test antibodies, CCK-8).

Four-parameter fitting of cell viability was performed using GraphPad Prism software based on the below formula: Y=(A−D)/[1+(X/C)B]+D.
A: Estimation of the asymptote above the curve;
D: Estimation of the asymptote under the curve;
B: The slope of the curve;
C: The dose corresponding to half of the maximum binding (EC50);
Y is the detected cell viability;
X is the drug concentration.

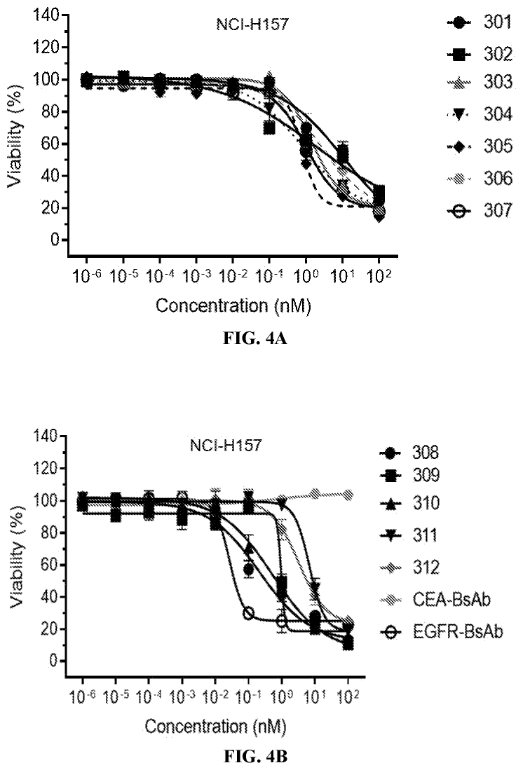

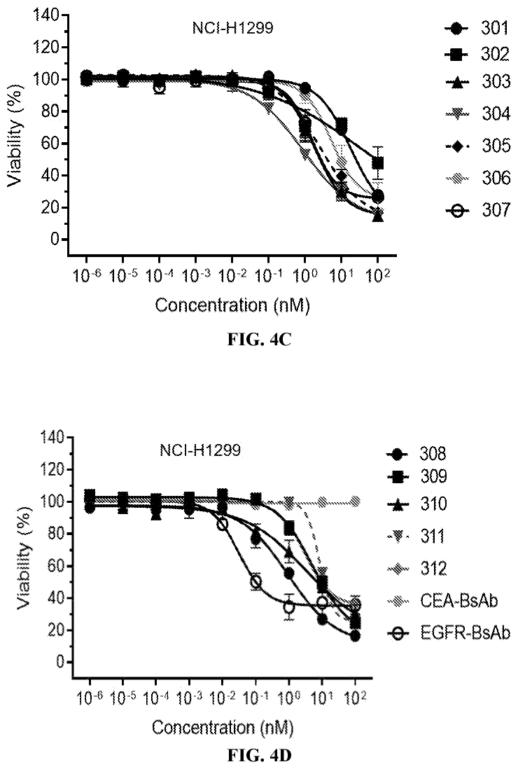

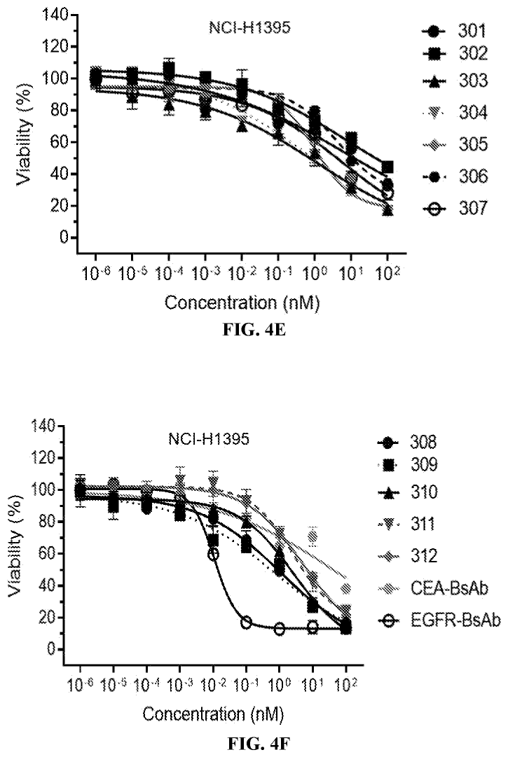

The results showed that trispecific antibodies induced PBMCs to kill tumor antigen-positive cells (FIGS. 4A-4F). EC50 values related to cell killing ability calculated using GraphPad Prism are shown in the below table. It can be seen that the cell killing effects of the non-unilateral CD3/CEA/EGFR trispecific antibody is significantly weaker than that of the anti-EGFR-BsAb bispecific antibody, but its cell killing effect is significantly stronger than that of the CEA-BsAb bispecific antibody.

TABLE 7
Killing effects of non-unilateral CD3/CEA/EGFR trispecific
antibodies on EGFR-positive tumor cells (EC50, nM)

| Sample | NCI-H157 | NCI-H1299 | NCI-H1395 |
|---|---|---|---|
| 301 | 11.33 | 17.82 | 8.211 |
| 302 | 1.779 | 26.06 | 6.478 |
| 303 | 1.462 | 1.745 | 1.148 |
| 304 | 0.7299 | 0.8946 | 0.6343 |
| 305 | 0.7595 | 2.598 | 0.9622 |
| 306 | 2.677 | 5.17 | 4.39 |
| 307 | 0.9831 | 1.561 | 5.559 |
| 308 | 0.2087 | 0.9503 | 1.082 |
| 309 | 0.9552 | 5.159 | 0.6949 |
| 310 | 0.5263 | 3.372 | 2.709 |
| 311 | 6.857 | 8.322 | 2.496 |
| 312 | 3.207 | 3.015 | 5.34 |
| CEA-BsAb | >100 | >100 | 43.14 |
| EGFR-BsAb | 0.02 | 0.03177 | 0.0108 |

### Example 5: Cytokine Detection after Non-Unilateral CD3/CEA/EGFR Trispecific Antibody Induces T Cells to Kill Tumor Cells

Cytokine secretion after T cell mediated cell killing of tumor cells induced by CD3/CEA/EGFR trispecific antibody was assessed by ELISA analysis of cell supernatants.

The CCK8 assay was performed as described above (Example 4) using an E:T ratio of 10:1 after incubation for 72 h. Subsequently, the 96-well plate was centrifuged at 2000 rpm for 10 min, and the supernatant was transferred to a new 96-well plate and stored at −20° C. for subsequent analysis. The amounts of TNFα, IFN-γ, IL-2 and IL-6 in the cell supernatant were determined using the R&D detection kits including: Human IL-2 DuoSet ELISA (R&D, #DY202-05), Human IL-6 DuoSet ELISA (R&D, #DY206-05), Human IFN-γ DuoSet ELISA (R&D, #DY285B-05), Human TNF-α DuoSet ELISA (R&D, #DY210).

The ELISA detection procedure is as follows: Capture Antibody was diluted with PBS 120 times to its working concentration. 100 μl of diluted antibody was added to each well of the 96-well sample plate. The 96-well plate was sealed, and incubated overnight at room temperature. The liquid was carefully aspirated from the 96-well plate. The 96-well plate was washed three times with 300 μl Wash Buffer, and pat-dried with tissue paper several times to absorb the liquid. 300 μl of Reagent Diluent was added to each well, and the wells were sealed and incubated at room temperature for 1 h. Samples and standards were prepared during the incubation period. The standards were diluted with Reagent Diluent at a 2-fold gradient dilution, with a total of 7 dilutions and a highest concentration of 1000 μg/ml. The supernatant of the cells to be tested were diluted 2.5 times with Reagent Diluent. After blocking, the plate was washed three times with 300 μl Wash Buffer and pat-dried with tissue paper several times to absorb the liquid. 100 μl of standard or sample was added to each well and the wells were incubated at room temperature for 2 h. The wells were then washed with 300 μl Wash Buffer three times and pat-dried. Reagent Diluent was used to dilute Detection Antibody 60 times to its working concentration. 100 μl of the diluted Detection Antibody was added to each well and incubated at room temperature for 2 h. The wells were washed three times with 300 μl Wash Buffer and pat-dried. Reagent Diluent was used to dilute Streptavidin-HRP 40 times to its working concentration. 100 μl of the diluted Streptavidin-HRP was added to each well and incubated at room temperature for 20 min. The wells were washed with 300 μl Wash Buffer three times and pat-dried. 100 μl Substrate Solution was added to each well. The wells were incubated in the dark at room temperature for 20 min. 50 μl Stop Solution was added to each well. The 96-well plate was tapped to mix the liquid, put into the microplate reader to read the optical density at 450 nm and 540 nm, respectively. OD450 reading value minus OD540 reading value was used as the final reading value. GraphPad software was used to fit the standard curve, and to calculate the concentration of the corresponding cytokine in the sample.

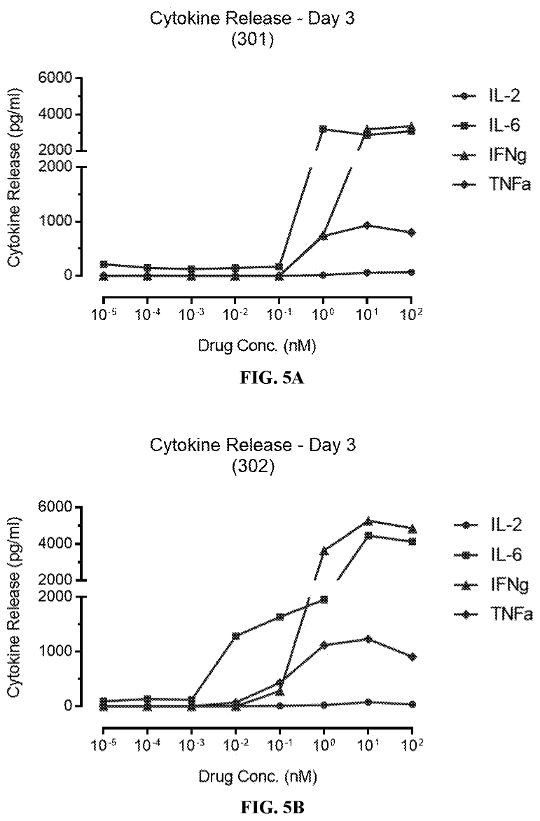

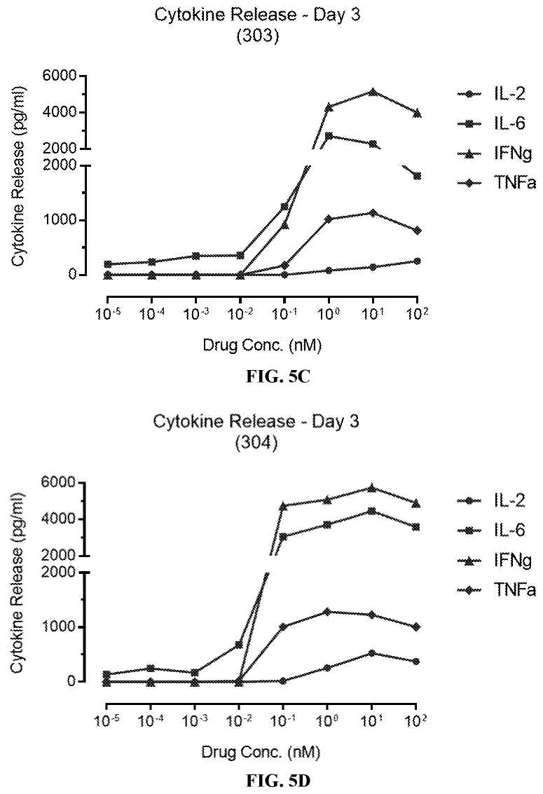

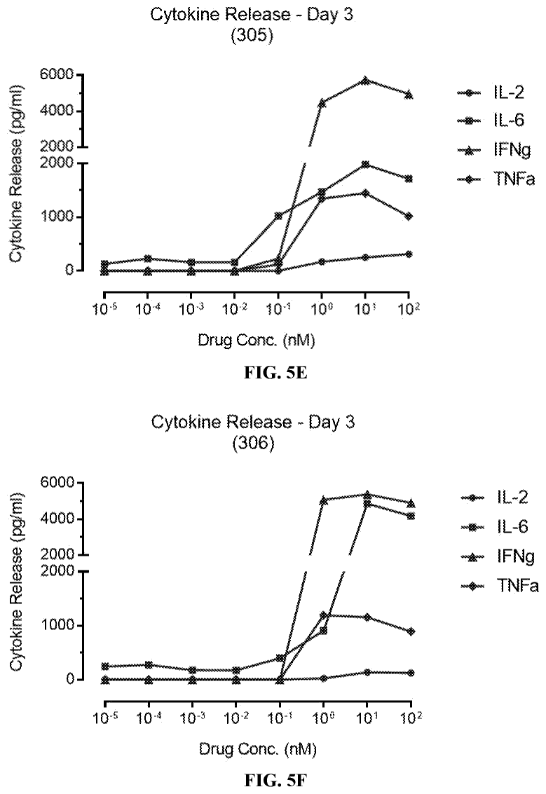

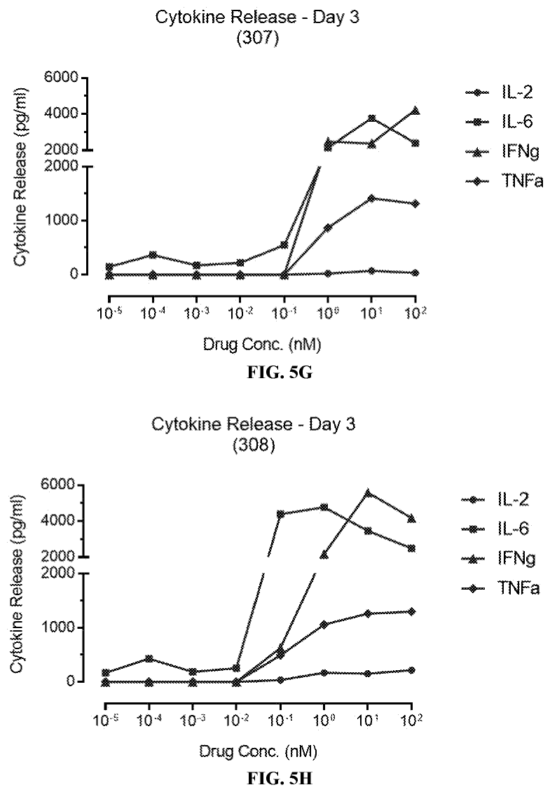

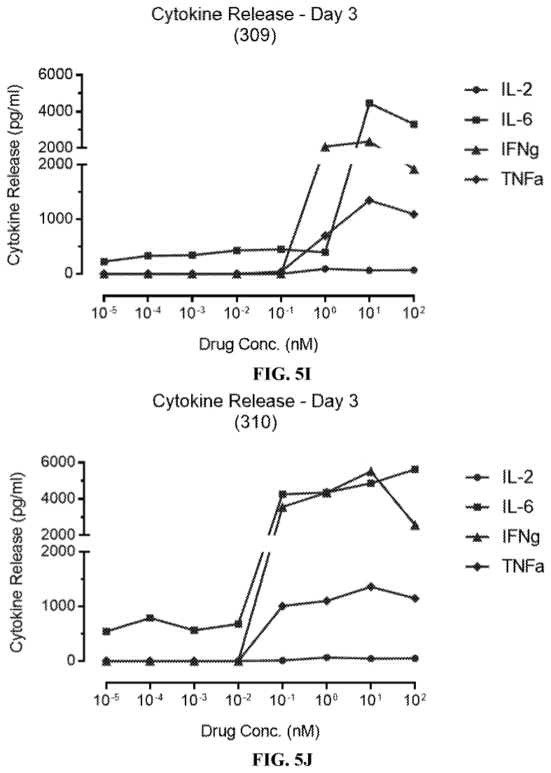

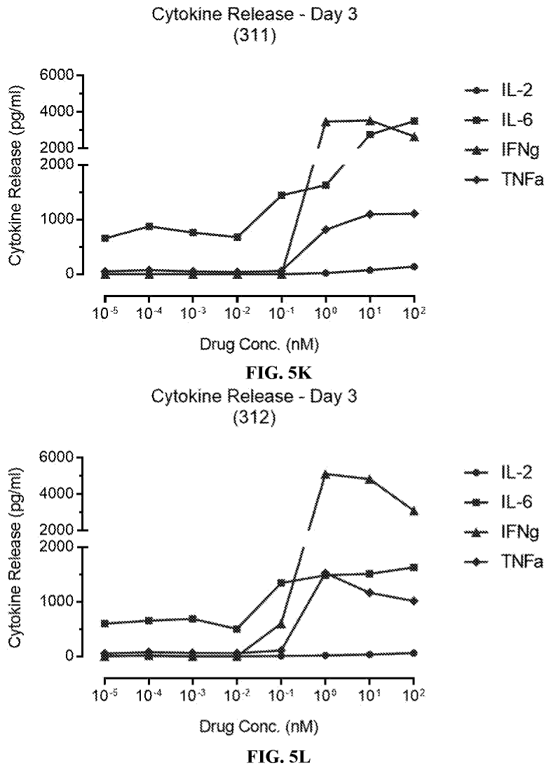

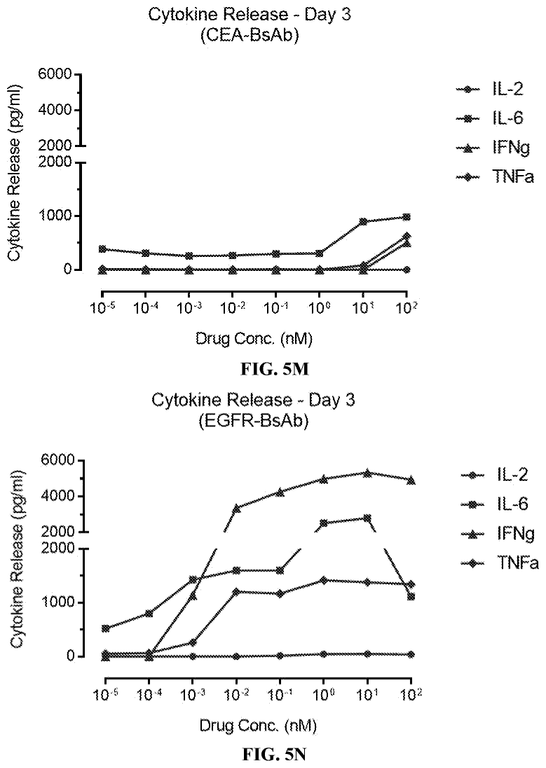

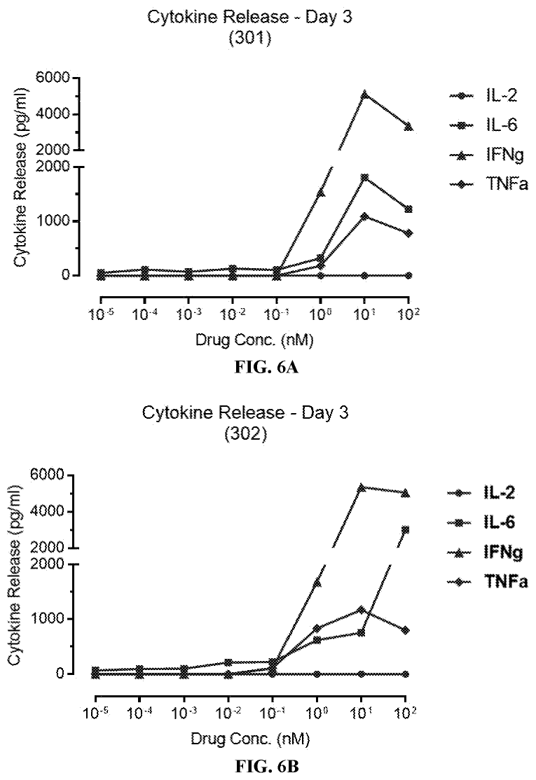

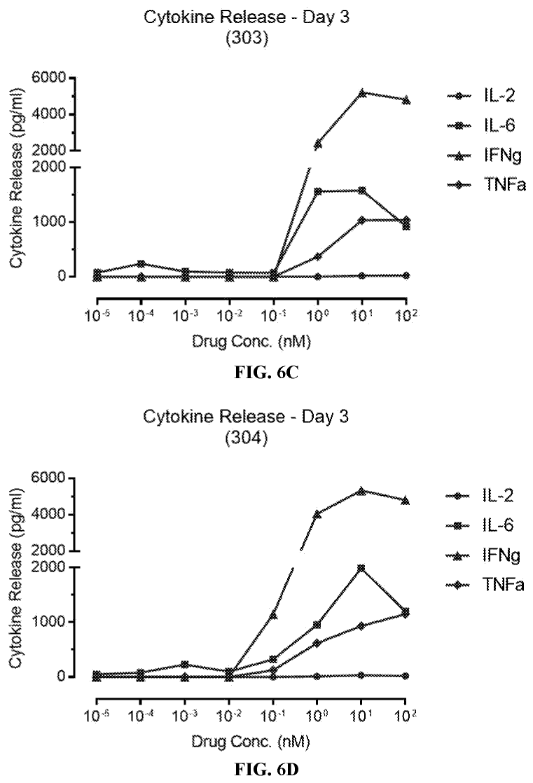

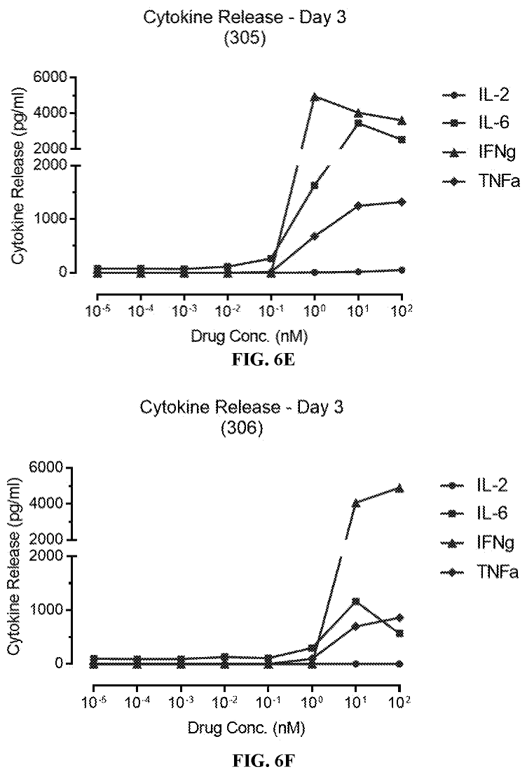

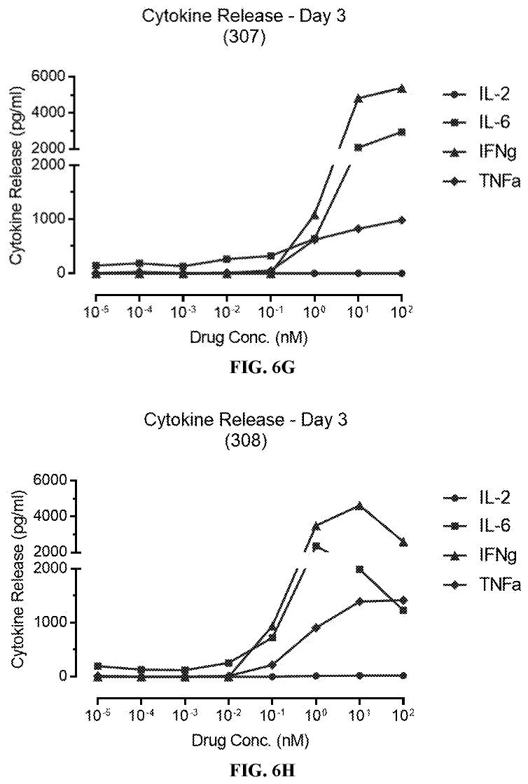

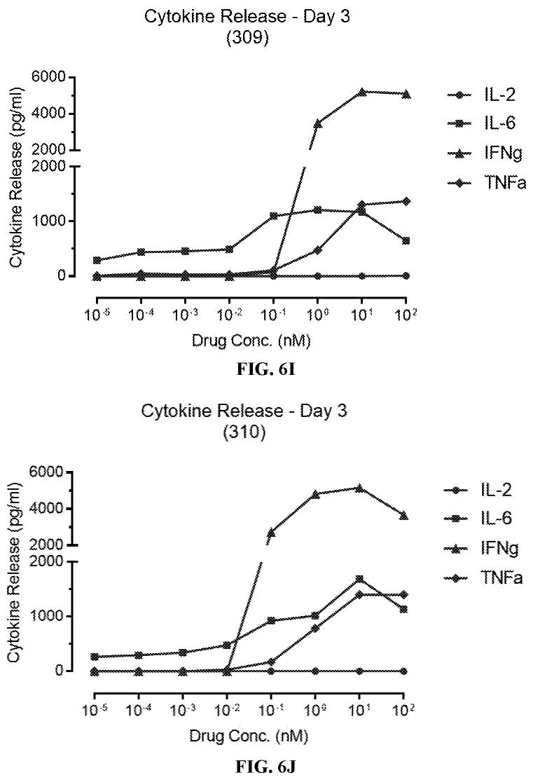

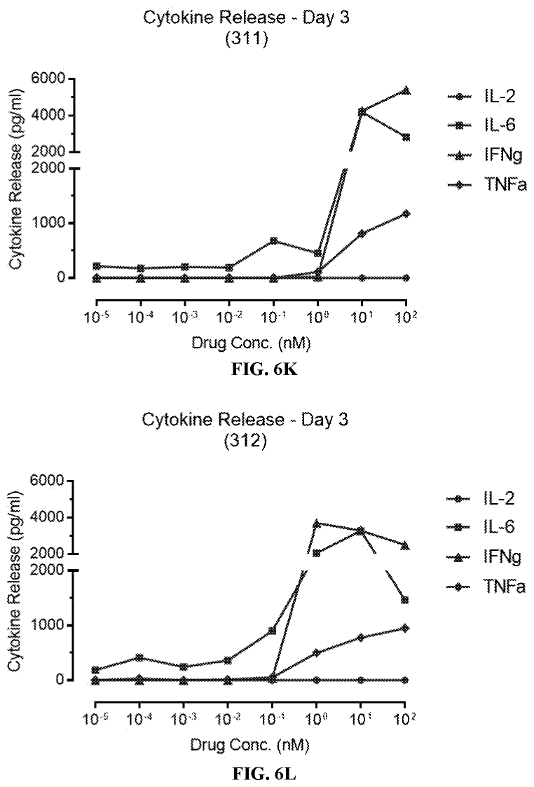

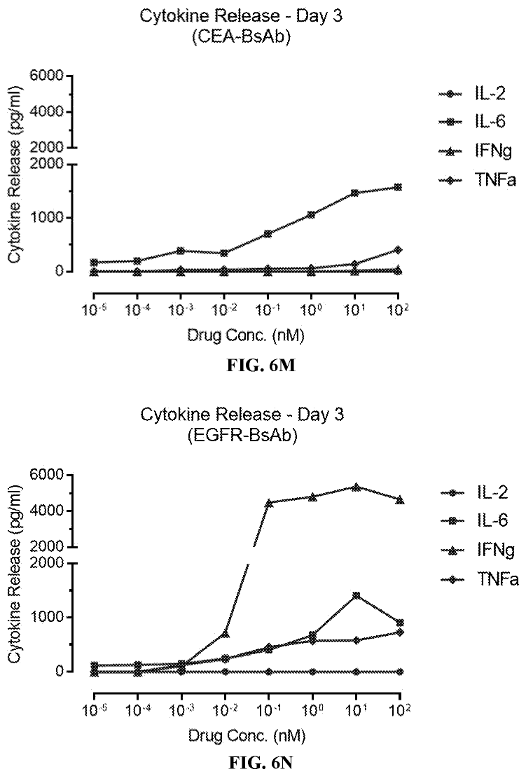

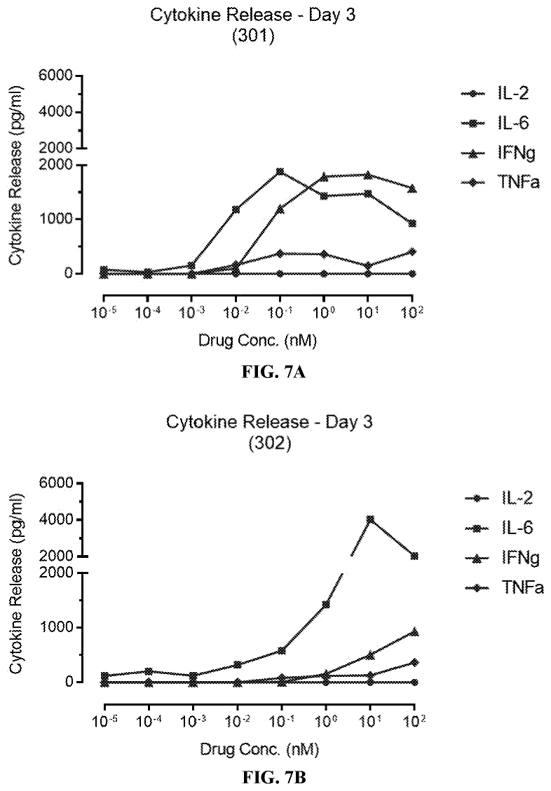

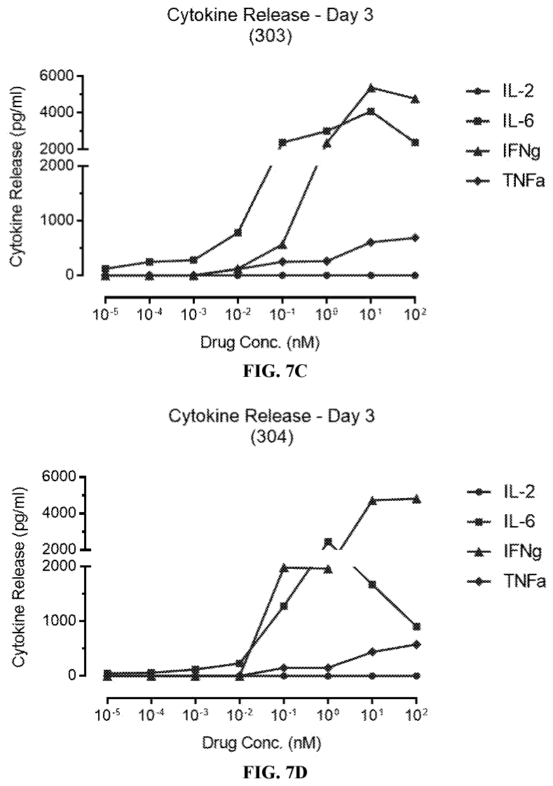

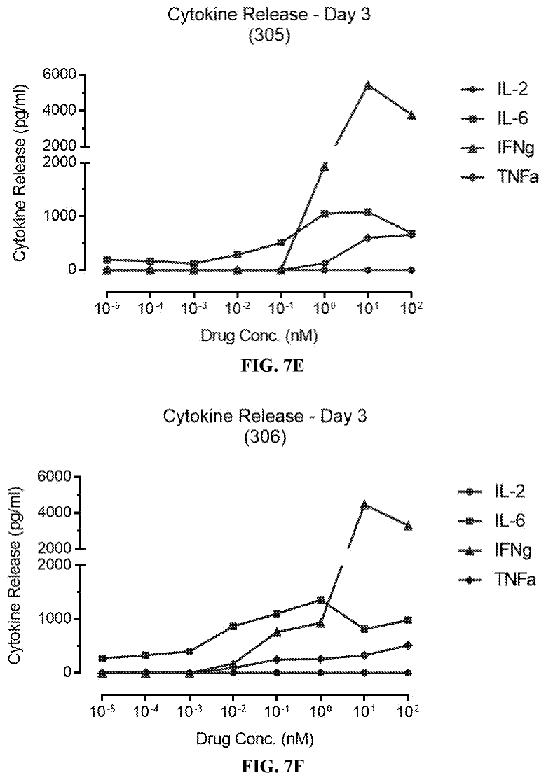

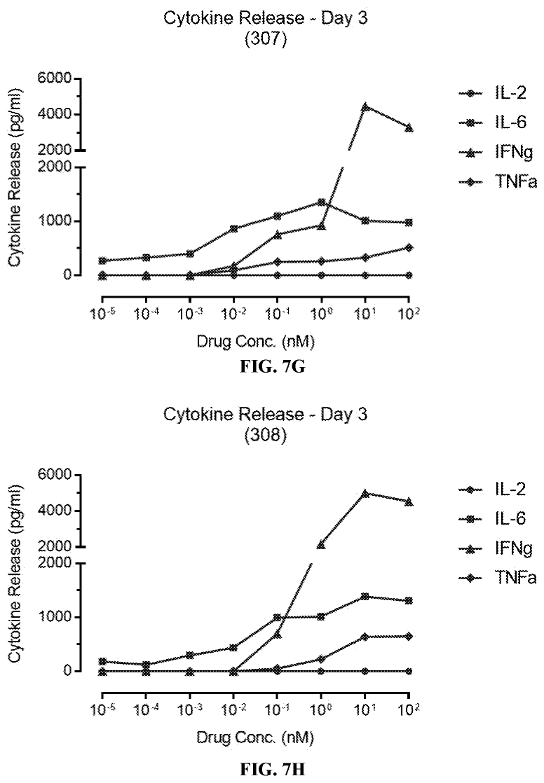

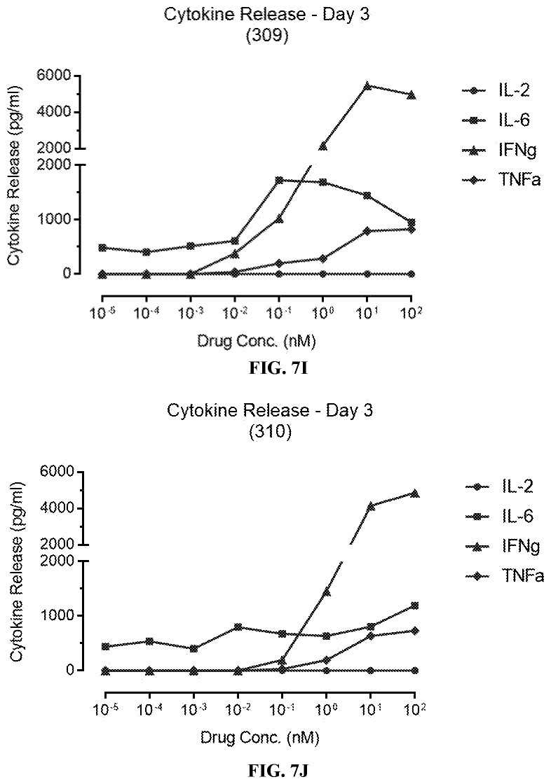

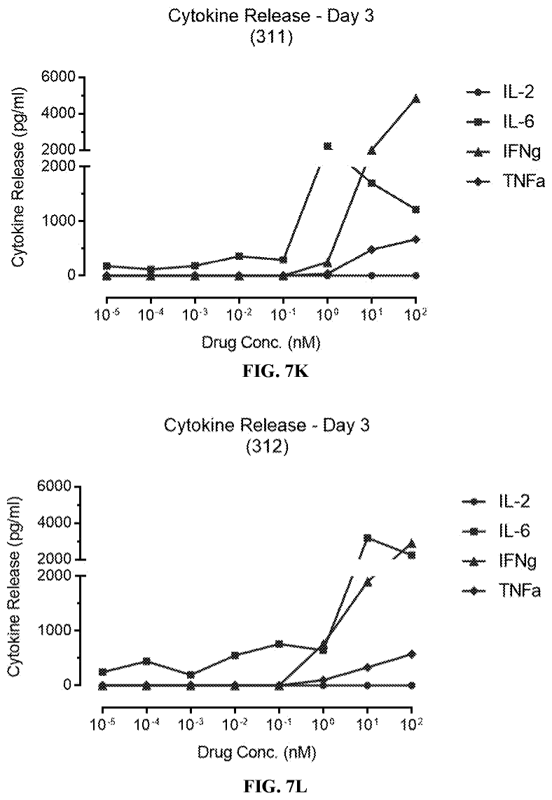

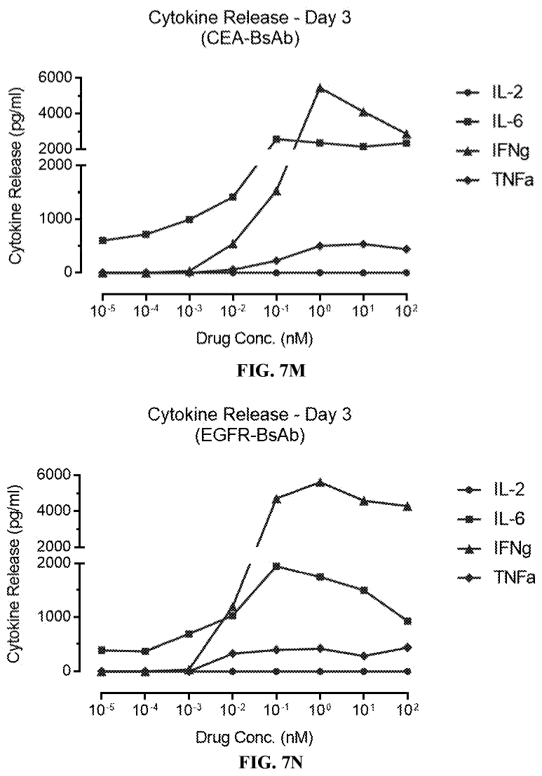

The cytokine content in the cell supernatant was detected by incubating the cells with the antibody for 72 h (see FIGS. 5-7). The results showed that the antibody significantly induced the release of cytokines during the antibody-induced T cell mediated cell killing. The amounts of released cytokines are ranked as below: INF-γ>IL-6>TNFα. IFN-γ secretion was significantly increased, and was closely related to cell killing. Under higher antibody concentrations, cells secreted significantly increased amounts of TNFα and IL-6, indicating potential adverse reactions (cytokine storm). Throughout the culture, IL-2 content did not change significantly.

In conclusion, these examples show that our CD3/CEA/EGFR tri-specific antibodies have significant T cell mediated cell killing effects. The secretion of IFN-γ was induced when the cells were killed, and the contents of TNFα and IL-6 were significantly increased, indicating that the safety of the non-unilateral trispecific antibodies should be monitored.

Based on the above results, the cell-killing effects of the non-unilateral CD3/CEA/EGFR trispecific antibodies were significantly weaker than that of EGFR-BsAb and stronger than that of CEA-BsAb.

### Example 6: Construction of Unilateral CD3/CEA/EGFR Trispecific Antibodies

According to FIG. 8, 16 unilateral tertiary antibody structures based on monovalent binding domains for CD3 (TA1-Fab), CEA and EGFR were designed. In these structures, TA1-Fab, EGFR single-domain antibody and CEA single-domain antibody are located at the N-terminus of Fe at the same time.

When the single-domain antibody is located at the C-terminus or N-terminus of the TA1 light chain, a Linker (SEQ ID NO: 11) is used to connect the two fragments. When the single-domain antibody is located at the C-terminus of the TA1 heavy chain, a Linker (SEQ ID NO: 11) is used to connect the two fragments. When the EGFR and CEA single domain antibodies are located at the knob-Fe or hole-Fe at the same time (FIGS. 8M-8P), a Linker (SEQ ID NO: 11) is used to connect the two single-domain antibodies, and the Fc hinge region (Hinge) acts as a linker to link the Fc and the single-domain antibody closer to the Fe.

Plasmid construction: All genes involved in this application were synthesized by GenScript (Nanjing GenScript Biotechnology Co., Ltd.), and then inserted into a mammalian expression vector pEE12.4 by enzyme digestion. The variable regions of the heavy and light chain DNA sequences were cloned into pEE12.4 in frame with pre-inserted human IgG4 heavy chain constant regions or kappa light chain constant regions. The plasmids were extracted with OMEGA's Plasmid Extraction Kit and stored at −80° C.

### Example 7: Preparation of Unilateral CD3/CEA/EGFR Trispecific Antibodies

The unilateral CD3/CEA/EGFR trispecific antibodies were generated by co-transfection of Expi 293F™ cells (ThermoFisher) with the above plasmids mediated by polyethyleneimine (PEI, POLYETHYLENEIMINE ‘MAX; polysciences, 24765-2). Cells were transfected with the corresponding expression plasmids in a 1:2:1 ratio [“heavy chain Fc(hole)”: “light chain”: “heavy chain Fc(knob)” ].

Expi 293F™ cells were cultured in suspension in CD OptiCHO™ medium, at 37° C., 5% CO₂, 135 rpm. One day before transfection, 293F cells were passaged into a 1 L gas-permeable conical flask (Corning), with a seeding density of 1.0×10⁶ cells/mL, and a volume of 200 mL. The estimated cell density on the day of transfection was 1.8-2.0×10⁶ cells/mL. The cell suspension was centrifuged at 1000 rpm for 5 min at room temperature, and washed once with Expi293 medium. The cells were collected and re-suspended in 200 mL Expi293 medium. 400 μg of plasmid was diluted with 5 mL of Opti-MEM medium, and vortexed for 15 s. 1.2 mg PEI was diluted with 5 mL Opti-MEM medium and vortexed for 15 s. The PEI-containing solution was added dropwise to the DNA-containing solution, mixed gently, and incubated at room temperature for 15 min. The plasmid/PEI mixture was added to the cell suspension. The cell suspension was placed in a 37° C., 5% CO₂, 85 rpm incubator for 4 hours. After 4 hours, 200 mL EX-CELL™293 medium and 2 mM Glutamine (Gibco) were added, and the rotation speed was adjusted to 135 rpm. After 24 hours, 3.8 mM VPA, a cell proliferation inhibitor, was added. After 72 hours, 40 mL medium D was added. After 7 days, the supernatant was collected by centrifugation at 18,000 rpm for 30 min. The supernatant was filtered with a 0.22 μm sterilization filter, and sodium azide was added to reach a final concentration of 0.01% w/v. The supernatant was stored at 4° C. for purification.

The target protein was purified using Protein A. Cell culture supernatants were loaded onto MabSelect Prism A FF (GE; 17-5498-01) resin equilibrated with 20 mL of equilibration buffer (25 mM Tris, 150 mM NaCl, pH 7.5). Unbound protein was removed by at least 10 column volumes of washing buffer (25 mM Tris, 150 mM NaCl, pH 7.5). The target protein was eluted with 5 column volumes of elution buffer (20 mM Na-Citrate, pH 3.5). The protein solution was neutralized by adding 1/10 volume of neutralization buffer (1 M Tris, pH 9.5).

The target protein was displaced into the desired buffer using Zeba™ desalting spin columns (ThermoFisher) or ultrafiltration tubes (Millipore). The protein concentration and purity were determined by SDS-PAGE and NanoDrop2000. 2-3 μg samples were used for SDS-PAGE electrophoresis.

The target protein was concentrated, filtered, and then applied to a gel filtration column (Gel Filtration, HiLoad Superdex 200, GE) equilibrated with equilibration buffer (20 mM histidine, 140 mM sodium chloride, pH 6.0). At room temperature, the samples were analyzed (for molecular weight, purity, aggregation, etc.) in PBS buffer (pH 7.2) at a flow rate of 0.5 mL/min. The final obtained high-purity monomeric protein was aliquoted and stored at −80° C.

In an alternative purification method, proteins were purified from cell supernatants by protein A affinity chromatography (MabSelect SuRe, GE). The protein eluate was then subjected to cation exchange chromatography (HiTrap SP HP, GE) and subsequently analyzed through a gel filtration column. The target protein obtained using this purification method was more than 90% pure.

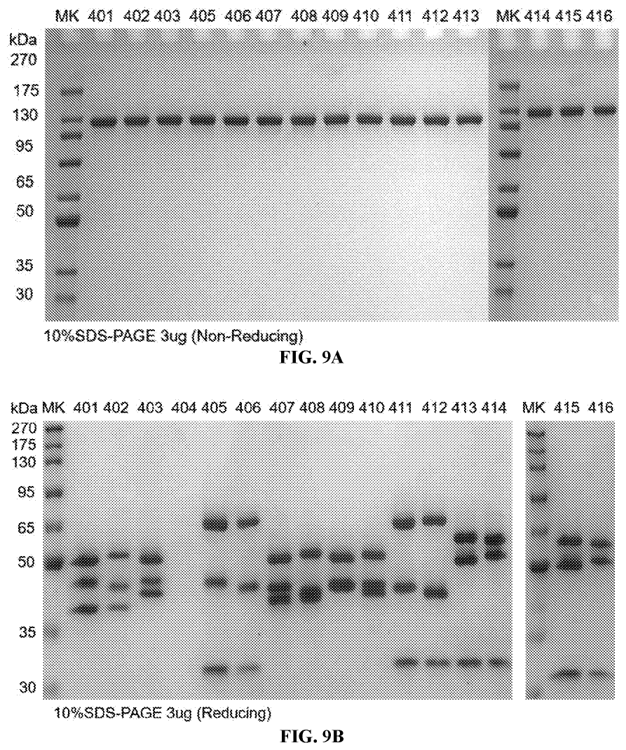

The protein SDS-PAGE results are shown in FIG. 9. The 404 protein was not successfully expressed. The yield of the tri-specific antibodies was 13.68-35.12 mg/L, and the purity was >92%. See the below table for details.

TABLE 8
Yield and purity of unilateral CD3/CEA/EGFR
trispecific antibodies

| Sample | Yield(mg/L) | Purity % |
|---|---|---|
| 401 | 22.96 | 97.12 |
| 402 | 13.92 | 95.36 |
| 403 | 16.32 | 94.28 |
| 404 | — | — |
| 405 | 19.36 | 93.11 |
| 406 | 13.68 | 95.32 |
| 407 | 24.16 | 94.73 |
| 408 | 16.24 | 93.59 |
| 409 | 25.84 | 96.53 |
| 410 | 15.52 | 95.12 |
| 411 | 18.96 | 96.21 |
| 412 | 35.12 | 94.78 |
| 413 | 26.64 | 95.31 |
| 414 | 17.76 | 94.45 |
| 415 | 26.64 | 96.25 |
| 416 | 14.04 | 94.52 |

### Example 8: Determining the Affinity of the Unilateral CD3/CEA/EGFR Trispecific Antibodies by Bio-Layer Interferometry (BLI)

Biofilm interferometry (BLI) experiments were performed on OCTECT RED96e (ForteBio) at 30° C. using 0.02% PBST (10 mmol/L Na₂HPO₄, 1.75 mmol/L KH₂PO₄, 137 mmol/L NaCl, 2.65 mmol/L KCl, pH 7.2-7.4, 0.02% surfactant Tween 20) as the running buffer.

Antibodies were first captured onto an anti-human IgG Fc coated AHC biosensor (Anti-hIgG Fc, Sartorius, 18-5060). Antibodies were diluted to 5 μg/mL and 1.5 nm protein was coupled to the AHC sensor surface in 0.02% PBST.

The analytes, including (1) recombinant human CD3ε/δ protein (ACRO systems, CDD-H52W1), (2) recombinant human tumor antigen CEA (CEACAM-5/CD66e, ACRO systems, CE5-H5226) and (3) recombinant human EGFR protein (ACRO systems, EGR-H5222), were diluted in 0.02% PBST to 200 nM, 100 nM, 50 nM, 25 nM, 12.5 nM, 6.25 nM, and 3.125 nM.

After the sensor captures the antibodies, it is used to bind and dissociate with different concentrations of analytes to obtain the KD value of the interaction. The experimental parameters are as follows: Baseline1: 60 s, Loading: 240 s, Baseline2: 180 s, Association: 240 s, Dissociation: 600 s, High sensitivity kinetics: 2 Hz. Kinetic constants were derived based on the rate equation for 1:1 Langmuir binding to obtain equilibrium dissociation constants (KD). The Data Analysis HT 12 software (Sartorius, 50-5029) was used to simultaneously fit binding and dissociation curves to calculate Kₒₙ, and K_{off}. The affinity KD was calculated based on the below formula: KD=K_{off}/Kₒₙ.

The experimental results showed that the affinity of the unilateral trispecific antibodies to CEA was 0.12 nM-0.90 nM, the affinity to EGFR was 6.87 nM-25.3 nM, and the affinity to CD3 was 0.013 nM-3.52 nM. The KD between trispecific antibodies and different antigens are shown in the below tables.

TABLE 9
Affinity of unilateral CD3/CEA/EGFR trispecific
antibodies to CEA (T = 30° C.)
Antigen: CEA

| Sample | ka (1 M⁻¹s⁻¹) | kdis (1/s) | KD (M) |
|---|---|---|---|
| 401 | 1.28E+05 | 1.15E−04 | 8.96E−10 |
| 402 | 9.24E+04 | 3.46E−05 | 3.75E−10 |
| 403 | 2.73E+05 | 3.26E−05 | 1.20E−10 |
| 405 | 2.30E+05 | 4.05E−05 | 1.76E−10 |
| 406 | 2.46E+05 | 6.17E−05 | 2.51E−10 |
| 407 | 1.85E+05 | 3.62E−05 | 1.96E−10 |
| 408 | 1.99E+05 | 4.73E−05 | 2.38E−10 |
| 409 | 6.10E+05 | 1.50E−04 | 2.46E−10 |
| 410 | 5.57E+05 | 8.03E−05 | 1.44E−10 |
| 411 | 5.47E+05 | 9.65E−05 | 1.77E−10 |
| 412 | 5.64E+05 | 7.94E−05 | 1.41E−10 |
| 413 | 7.10E+05 | 1.12E−04 | 1.57E−10 |
| 414 | 6.57E+05 | 1.05E−04 | 1.60E−10 |
| 415 | 4.31E+05 | 7.70E−05 | 1.79E−10 |
| 416 | 4.21E+05 | 7.97E−05 | 1.89E−10 |
| CEA-BsAb | 2.26E+05 | 7.84E−05 | 3.47E−10 |

TABLE 10
Affinity of unilateral CD3/CEA/EGFR trispecific
antibodies to EGFR (T = 30° C.)
Antigen: EGFR

| Sample | ka (1 M⁻¹s⁻¹) | kdis (1/s) | KD (M) |
|---|---|---|---|
| 401 | 3.45E+05 | 2.87E−03 | 8.33E−09 |
| 402 | 3.65E+05 | 2.51E−03 | 6.87E−09 |
| 403 | 3.34E+05 | 3.45E−03 | 1.03E−08 |
| 405 | 3.27E+05 | 3.22E−03 | 9.87E−09 |
| 406 | 3.41E+05 | 3.35E−03 | 9.83E−09 |
| 407 | 1.56E+05 | 3.94E−03 | 2.53E−08 |
| 408 | 1.97E+05 | 4.67E−03 | 2.37E−08 |
| 409 | 3.87E+05 | 3.49E−03 | 9.03E−09 |
| 410 | 3.57E+05 | 3.39E−03 | 9.48E−09 |
| 411 | 3.54E+05 | 3.55E−03 | 1.00E−08 |
| 412 | 3.82E+05 | 3.56E−03 | 9.31E−09 |
| 413 | 2.77E+05 | 4.14E−03 | 1.49E−08 |
| 414 | 2.76E+05 | 4.46E−03 | 1.62E−08 |
| 415 | 3.90E+05 | 4.05E−03 | 1.04E−08 |
| 416 | 4.15E+05 | 4.22E−03 | 1.02E−08 |
| EGFR-BsAb | 3.09E+05 | 3.05E−03 | 9.85E−09 |

TABLE 11
Affinity of unilateral CD3/CEA/EGFR trispecific
antibodies to CD3ε/δ (T = 30° C.)
Antigen: CD3ε/δ

| Sample | ka (1 M⁻¹s⁻¹) | kdis (1/s) | KD (M) |
|---|---|---|---|
| 401 | 1.73E+05 | 1.96E−04 | 1.14E−09 |
| 402 | 4.08E+05 | 2.60E−05 | 6.38E−11 |
| 403 | 5.03E+05 | 2.18E−04 | 4.34E−10 |
| 405 | 5.04E+05 | 2.26E−04 | 4.49E−10 |
| 406 | 4.58E+05 | 1.42E−04 | 3.11E−10 |
| 407 | 4.23E+05 | 5.43E−06 | 1.28E−11 |
| 408 | 4.16E+05 | 8.43E−06 | 2.03E−11 |
| 409 | 1.52E+05 | 4.16E−04 | 2.74E−09 |
| 410 | 4.98E+05 | 7.72E−05 | 1.55E−10 |
| 411 | 1.24E+05 | 4.37E−04 | 3.52E−09 |
| 412 | 5.18E+05 | 9.11E−05 | 1.76E−10 |
| 413 | 1.58E+05 | 3.19E−04 | 2.03E−09 |
| 414 | 5.77E+05 | 1.94E−04 | 3.36E−10 |
| 415 | 1.44E+05 | 2.11E−04 | 1.46E−09 |
| 416 | 6.15E+05 | 2.52E−04 | 4.09E−10 |
| CEA-BsAb | 4.81E+05 | 2.36E−04 | 4.91E−10 |
| EGFR-BsAb | 8.29E+04 | 7.48E−04 | 9.03E−9 |

### Example 9: Unilateral CD3/CEA/EGFR Trispecific Antibodies Induce T Cell Mediated Killing of EGFR-Positive Tumor Target Cells

T cell mediated killing of target cells induced by trispecific antibodies was tested in human tumor cells NCI-H1299 (CEA negative; medium levels of EGFR), NCI-H157 (CEA negative; medium levels of EGFR), NCI-H1395 (medium levels of CEA; medium levels of EGFR) and NCI-H1573 (high levels of CEA; high levels of EGFR). Human PBMCs were used as effectors and killing was detected after 72 h incubation with antibodies.

Briefly, tumor cells were digested with trypsin/EDTA, washed once with pre-chilled PBS, re-suspended in RPMI 1640 medium containing 10% FBS, and plated at a density of 5,000 cells/well in a flat-bottom 96-well plate (Corning 3599). After culturing for 4 h, 50 μL of serially diluted antibody solution was added to each well. Each concentration was added to three replicate wells. The antibodies were incubated with the tumor cells for more than 30 min to fully attach the proteins to the cells. PBMC cells (Reid Bio, 1521) were thawed quickly, resuscitated, added to 10 mL of RPMI 1640 medium containing 10% FBS, centrifuged at 1000 rpm for 5 min, and re-suspended after discarding the supernatant. Cell density was adjusted and 50 μL of PBMCs was added to each well of target cells to reach a final E:T ratio of 10:1. The 96-well plate was placed in a 37° C., 5% CO₂ incubator for 3 days.

After culturing for 3 days, the cell culture supernatant was carefully removed. The plate was pat dried with a paper towel. Serum-Free DMEM containing 10% CCK8 detection solution (Dojindo, CK04-20) was added to the 96-well plate and incubated at 37° C. for 2-4 hours. After 4 hours, the absorbance at 450 nm was detected using a microplate reader (BioTech, Synergy LX). The cell viability was calculated according to the below formula:

cell viability=[(As−Ab)/(Ac−Ab)]×100%.

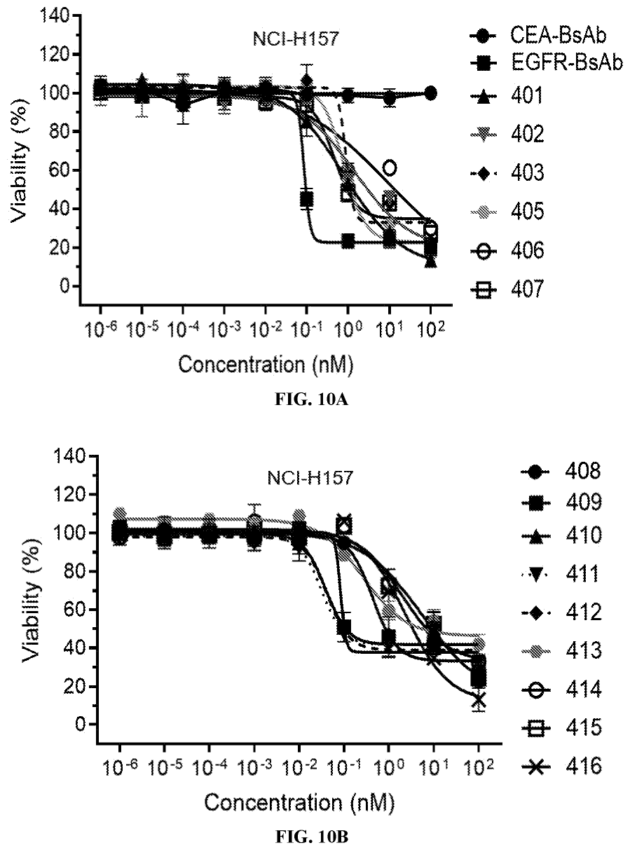

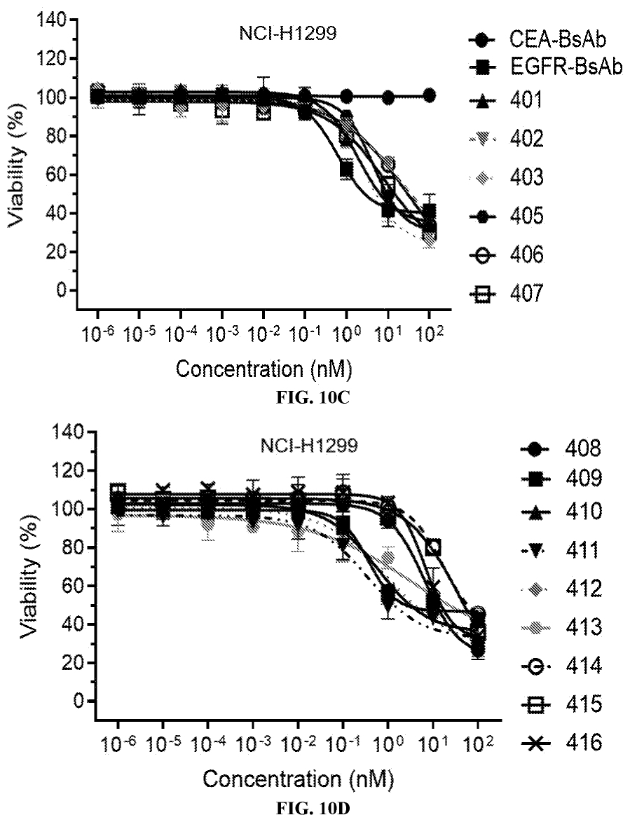

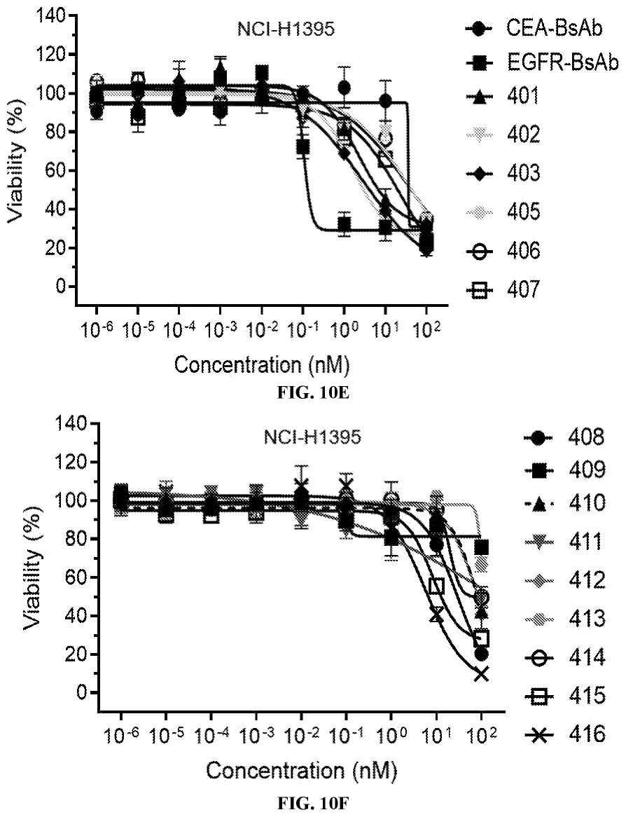

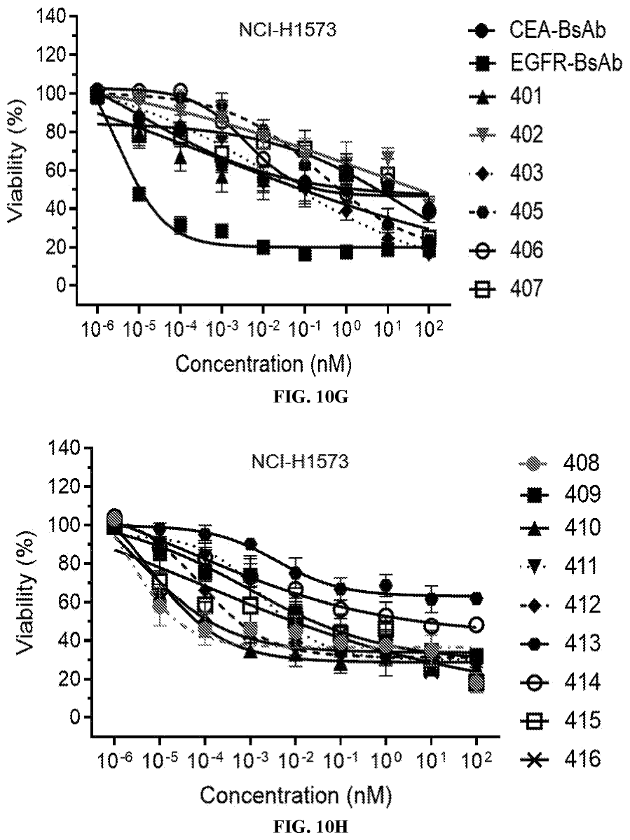

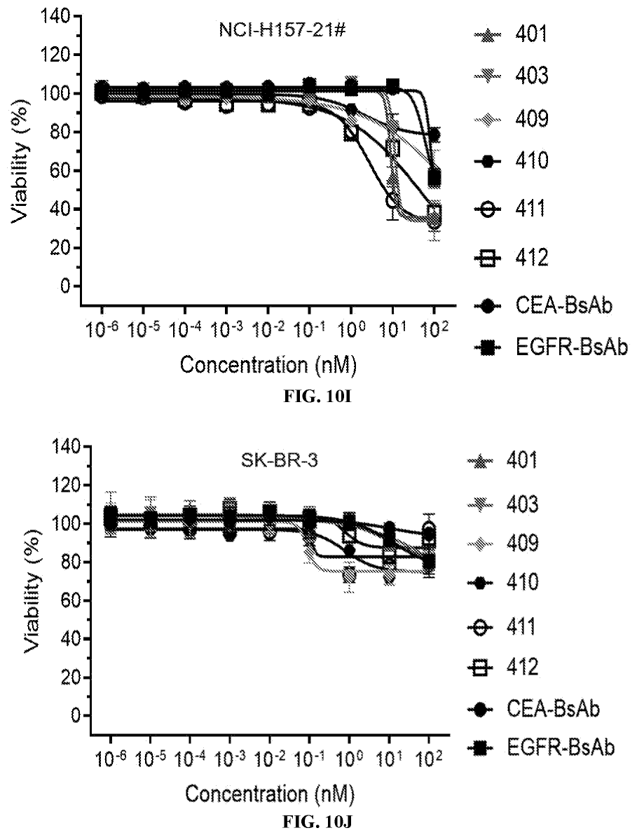

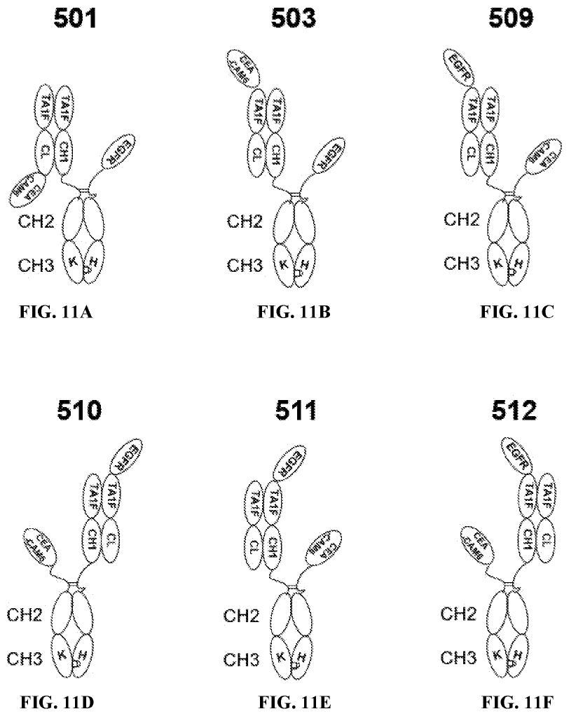

The results showed that the unilateral trispecific antibodies induced PBMC to kill the tumor antigen EGFR-positive cells (FIG. 10). EC50 values related to cell killing ability calculated using GraphPad Prism are shown in the below table.

It can be seen that the killing effects of the unilateral trispecific antibodies on tumor cells NCI-H157 and NCI-H1299 were significantly stronger than that of CEA-BsAb bispecific antibody. The killing effects of 409-412 were stronger than or close to that of EGFR-BsAb bispecific antibody. Except for 410-414, the killing effect of unilateral trispecific antibodies on tumor cell NCI-H1395 was significantly stronger than or close to that of CEA-BsAb. The killing effect of 409 was significantly stronger than that of EGFR-BsAb. Unilateral trispecific antibodies 401, 406, 408-411, and 416 had significantly stronger killing effects on tumor cells NCI-H1573 than CEA-BsAb and their killing effects were close to that of EGFR-BsAb.

TABLE 12
Unilateral CD3/CEA/EGFR trispecific antibodies induce Killing
effect of on EGFR-positive tumor cells (EC50, nM)

| Sample | NCI-H157 | NCI-H1299 | NCI-H1395 | NCI-H1573 |
|---|---|---|---|---|
| 401 | 0.8232 | 1.881 | 2.311 | 4.57E−06 |
| 402 | 1.461 | 24.24 | 42.85 | 21.2 |
| 403 | 0.8891 | 2.761 | 2.796 | 7.62E−03 |
| 405 | 0.7917 | 3.715 | 1.542 | 0.2379 |
| 406 | 8.936 | 30.33 | 38.91 | 3.84E−06 |
| 407 | 0.4654 | 8.017 | 23.7 | 21.12 |
| 408 | 0.4313 | 6.546 | 31.9 | 2.72E−06 |
| 409 | 0.08364 | 0.3672 | 0.0988 | 3.17E−06 |
| 410 | 0.03922 | 0.5909 | 78.35 | 4.52E−06 |
| 411 | 0.03178 | 0.3548 | 128.8 | 1.93E−06 |
| 412 | 0.04142 | 1.051 | 74.51 | 1.16E−05 |
| 413 | 0.2708 | 3.401 | >100 | 4.35E−05 |
| 414 | 2.001 | 16.1 | 100 | 1.11E−05 |
| 415 | 5.517 | 42.26 | 9.867 | 7.38E−05 |
| 416 | 2.437 | 6.728 | 6.415 | 2.14E−06 |
| CEA-BsAb | >100 | >100 | 36.01 | 1.16E−05 |
| EGFR-BsAb | 0.08559 | 0.6153 | 0.1067 | 2.89E−06 |

### Example 10: Unilateral CD3/CEA/EGFR Trispecific Antibodies Induce T Cell Mediated Killing of EGFR-Positive Tumor Target Cells with Low EGFR Expression

Unilateral CD3/CEA/EGFR trispecific antibodies 401, 403, 409, 410, 411 and 412 were evaluated for their ability to induce T-cell mediated killing on EGFR-low expressing tumor target cells. The tumor cell lines used include: human breast adenocarcinoma SK-BR-3 (EGFR negative), human non-small cell lung adenocarcinoma NCI-H157 monoclonal 21 cell line (NCI-H157-21#) (EGFR knockdown) (iGene Biotechnology, HSH117865-LVRU6GP).

Killing was detected after 72 h incubation with antibodies using human PBMCs as effectors. EC50 values related to cell killing ability calculated using GraphPad Prism are shown in the table below.

The results showed that the unilateral CD3/CEA/EGFR trispecific antibodies 410-412 significantly enhanced the killing of T cells against EGFR low-expressing tumor cells (FIG. 10), and its killing effect was significantly stronger than that of CEA-BsAb and EGFR-BsAb bispecific control antibodies. In addition, the killing effect of 409 on SK-BR-3 cells was significantly stronger than that of CEA-BsAb and EGFR-BsAb. The killing effects of 401 and 403 on NCI-H157-21# cells were significantly stronger than that of CEA-BsAb and EGFR-BsAb. Since SK-BR-3 and NCI-H157-21# are both cells with low CEA expression, the unilateral CD3/CEA/EGFR trispecific antibodies 401, 403, 409, 410, 411 and 412 have potential application values in cells with low CEA and EGFR expressions.

TABLE 13
Killing effects of unilateral CD3/CEA/EGFR trispecific antibodies
on tumor cells with low EGFR expression (EC50, nM)

| Sample | SK-BR-3 | NCI-H157-21# |
|---|---|---|
| 401 | >100 | 8.966 |
| 403 | >100 | 11.35 |
| 409 | 0.0884 | >100 |
| 410 | 0.7919 | 2.096 |
| 411 | 0.1146 | 2.74 |
| 412 | 0.7869 | 47.84 |
| CEA-BsAb | >100 | 60.83 |
| EGFR-BsAb | >100 | 79.13 |

### Example 11: Preparation of Unilateral CD3/CEA-CAM6/EGFR, CD3/CEA/HER2 and CD3/CEA-CAM6/HER2 Trispecific Antibodies

HER2 and CEA-CAM6 single domain antibodies were obtained by immunizing llamas (lama). Following Example 6, based on the structures of 401, 403, 409-412, we designed unilateral CD3/CEA-CAM6/EGFR, CD3/CEA/HER2 and CD3/CEA-CAM6/HER2 trispecific antibodies, named respectively as 501, 503, 509-512; 601, 603, 609-612; 701, 703, 709-712 (FIG. 11). The sequences of HER2 and CEA-CAM6 single domain antibodies are shown in the table below.

TABLE 14
HER2, CEA-CAM6 single domain antibody sequences, linker sequence and
Fc hinge region sequence, linker sequence and Fc hinge region sequence

| | SEQ ID | |
|---|---|---|
| Name | NO. | Sequence |
| HER2(VHH) | 9 | QVQLVQSGGGLVQAGGSLRLSCAASGRTFSSYAMAWFRQAPGKEREFVA |
| | | AISWSGANIYVADSVKGRFTISRDNAKDTVYLQMNSLKPEDTAVYYCAVK |
| | | LGFAPVEERQYDYWGQGTQVTVSS |
| CEA-CAM6 | 10 | QVKLEESGGGLVQAGGSLRLSCRTSGRTNSVYTMGWFRQAPGKEREFVA |
| (VHH) | | QIMWGAGTNTHYADSVKGRFTISRDSAESTVYLQMNSLKPEDTAVYYCA |
| | | ANRGIPIAGRQYDYWGQGTQVTVSS |
| Linker | 11 | GGGGSGGGGS |
| Fc hinge | 12 | ESKYGPPCPPCP |
| region | | |
| (Hinge) | | |

Plasmid construction: All genes involved in this application were synthesized by GenScript (Nanjing GenScript Biotechnology Co., Ltd.), and then inserted into a mammalian expression vector pEE12.4 by enzyme digestion. The variable regions of the heavy and light chain DNA sequences were cloned into pEE12.4 in frame with pre-inserted human IgG4 heavy chain constant regions or kappa light chain constant regions. The plasmids were extracted with OMEGA's Plasmid Extraction Kit and stored at −80° C.

Following Examples 2 and 7, the above plasmids were transfected into Expi 293F™ cells by PEI, and antibodies were prepared by various purification methods.

The protein SDS-PAGE results are shown in FIG. 12. The yield of the trispecific antibodies is 13.18-45.1 mg/L, and the purity is >92%. See the below table for details.

TABLE 15
Yield and purity of unilateral CD3/CEA-CAM6/EGFR, CD3/CEA/HER2
and CD3/CEA-CAM6/HER2 trispecific antibodies

| Sample | Yield(mg/L) | Purity % |
|---|---|---|
| 501 | 35.62 | 95.34 |
| 503 | 45.1 | 95.82 |
| 509 | 32.26 | 92.91 |
| 510 | 16.48 | 96.11 |
| 511 | 22.12 | 95.61 |
| 512 | 19.24 | 92.82 |
| 601 | 21.04 | 92.22 |
| 603 | 23.08 | 95.52 |
| 609 | 24.1 | 95.63 |
| 610 | 19.54 | 95.71 |
| 611 | 19.54 | 95.22 |
| 612 | 20.92 | 95.84 |
| 701 | 32.5 | 95.74 |
| 703 | 22.9 | 93.57 |
| 709 | 23.26 | 92.76 |
| 710 | 13.18 | 95.66 |
| 711 | 17.26 | 96.31 |
| 712 | 17.2 | 95.93 |

### Example 12: Determining the Affinity of Unilateral CD3/CEA-CAM6/EGFR, CD3/CEA/HER2 and CD3/CEA-CAM6/HER2 Trispecific Antibodies by BLI

Biofilm interferometry (BLI) experiments were performed on OCTECT RED96e (ForteBio) at 30° C. using 0.02% PBST (10 mmol/L Na₂HPO₄, 1.75 mmol/L KH₂PO₄, 137 mmol/L NaCl, 2.65 mmol/L KCl, pH 7.2-7.4, 0.02% surfactant Tween 20) as the running buffer.

Antibodies were first captured onto an anti-human IgG Fc coated AHC biosensor (Anti-hIgG Fc, Sartorius, 18-5060). Antibodies were diluted to 5 μg/mL and 1.5 nm protein was coupled to the AHC sensor surface in 0.02% PBST.

The analytes, including (1) recombinant human protein CD3ε/δ (ACRO systems, CDD-H52W1), (2) recombinant human tumor antigen CEA (CEACAM-5/CD66e, ACRO systems, CE5-H5226), (3) recombinant human EGFR protein (ACRO systems, EGR-H5222), (4) recombinant human tumor antigen HER2 (ERBB2, ACRO systems, HE2-H5212) and (5) recombinant human tumor antigen CEACAM-6 (ACRO systems, CE6-H5223) were diluted in 0.02% PBST to 200 nM, 100 nM, 50 nM, 25 nM, 12.5 nM, 6.25 nM, and 3.125 nM.

After the sensor captures the antibodies, it binds and dissociates with different concentrations of analytes to obtain the KD value of the interaction. The experimental parameters are as follows: Baseline1: 60 s, Loading: 240 s, Baseline2: 180 s, Association: 240 s, Dissociation: 600 s, High sensitivity kinetics: 2 Hz. Kinetic constants were derived based on the rate equation for 1:1 Langmuir binding to obtain equilibrium dissociation constants (KD). The Data Analysis HT 12 software (Sartorius, 50-5029) was used to simultaneously fit binding and dissociation curves to calculate Kₒₙ and K_{off}. The affinity KD was calculated based on the below formula: KD=K_{off}/Kₒₙ.

The experimental results showed that the unilateral CD3/CEA-CAM6/EGFR trispecific antibodies had an affinity of 0.634 nM-2.01 nM with CEA-CAM6, 2.65 nM-11.3 nM with EGFR, and 6.09 nM-7.47 nM with CD3. The KD for different antigens are shown in the tables below.

TABLE 16
Affinity of unilateral CD3/CEA-CAM6/EGFR trispecific
antibodies to CEA-CAM6 (T = 30° C.)
Antigen: CEA-CAM6

| Sample | ka (1 M⁻¹s⁻¹) | kdis (1/s) | KD (M) |
|---|---|---|---|
| 501 | 3.21E+05 | 2.04E−04 | 6.34E−10 |
| 503 | 2.27E+05 | 2.68E−04 | 1.18E−09 |
| 509 | 1.71E+05 | 3.28E−04 | 1.91E−09 |
| 510 | 1.47E+05 | 1.67E−04 | 1.14E−09 |
| 511 | 2.55E+05 | 2.96E−04 | 1.16E−09 |
| 512 | 2.72E+05 | 5.47E−04 | 2.01E−09 |
| EGFR-BsAb | 2.22E+05 | 2.47E−04 | 1.11E−09 |
| CEACAM6-BsAb | 2.06E+05 | 3.36E−04 | 1.64E−09 |

TABLE 17
Affinity of unilateral CD3/CEA-CAM6/EGFR trispecific
antibodies to EGFR (T = 30° C.)
Antigen: EGFR

| Sample | ka (1 M⁻¹s⁻¹) | kdis (1/s) | KD (M) |
|---|---|---|---|
| 501 | 2.06E+05 | 2.34E−03 | 1.13E−08 |
| 503 | 5.51E+05 | 1.46E−03 | 2.65E−09 |
| 509 | 5.26E+05 | 1.69E−03 | 3.21E−09 |
| 510 | 4.40E+05 | 1.30E−03 | 2.94E−09 |
| 511 | 5.85E+05 | 1.80E−03 | 3.08E−09 |
| 512 | 5.81E+05 | 1.58E−03 | 2.72E−09 |
| EGFR-BsAb | 1.17E+06 | 5.19E−03 | 4.43E−09 |
| CEA-CAM6-BsAb | 5.70E+05 | 1.43E−03 | 2.52E−09 |

TABLE 18
Affinity of unilateral CD3/CEA-CAM6/EGFR trispecific
antibodies to CD3ε/δ (T = 30° C.)
Antigen: CD3ε/δ

| Sample | ka (1 M⁻¹s⁻¹) | kdis (1/s) | KD (M) |
|---|---|---|---|
| 501 | 5.80E+05 | 3.53E−03 | 6.09E−09 |
| 503 | 5.47E+05 | 3.39E−03 | 6.20E−09 |
| 509 | 5.09E+05 | 3.80E−03 | 7.47E−09 |
| 510 | 4.99E+05 | 3.49E−03 | 6.99E−09 |
| 511 | 5.53E+05 | 4.11E−03 | 7.45E−09 |
| 512 | 5.74E+05 | 3.98E−03 | 6.94E−09 |
| EGFR-BsAb | 5.26E+05 | 3.38E−03 | 6.43E−09 |
| CEA-CAM6-BsAb | 7.34E+05 | 4.21E−03 | 5.73E−09 |

The experimental results showed that the unilateral CD3/CEA/HER2 trispecific antibodies had an affinity of 0.107 nM-0.195 nM with CEA, an affinity of 48.4 nM-108 nM with HER2, and an affinity of 0.608 nM-1.77 nM with CD3. The KD data are shown in the tables below.

TABLE 19
Affinity of unilateral CD3/CEA-CAM6/EGFR trispecific
antibodies to CEA (T = 30° C.)
Antigen: CEA

| Sample | ka (1 M⁻¹s⁻¹) | kdis (1/s) | KD (M) |
|---|---|---|---|
| 601 | 5.72E+05 | 8.52E−05 | 1.49E−10 |
| 603 | 2.10E+05 | 2.47E−05 | 1.18E−10 |
| 609 | 4.71E+05 | 5.04E−05 | 1.07E−10 |
| 610 | 5.25E+05 | 8.05E−05 | 1.53E−10 |
| 611 | 5.53E+05 | 1.08E−04 | 1.95E−10 |
| 612 | 5.42E+05 | 1.05E−04 | 1.93E−10 |
| CEA-BsAb | 5.13E+05 | 6.35E−05 | 1.24E−10 |
| HER2-BsAb | 8.77E+05 | 3.91E−04 | 4.46E−10 |

TABLE 20
Affinity of unilateral CD3/CEA-CAM6/EGFR trispecific
antibodies to HER2 (T = 30° C.)
Antigen: HER2

| Sample | ka (1 M⁻¹s⁻¹) | kdis (1/s) | KD (M) |
|---|---|---|---|
| 601 | 1.35E+05 | 1.35E−02 | 1.00E−07 |
| 603 | 1.39E+05 | 1.05E−02 | 7.59E−08 |
| 609 | 1.66E+05 | 8.03E−03 | 4.84E−08 |
| 610 | 1.71E+05 | 9.14E−03 | 5.35E−08 |
| 611 | 1.11E+05 | 1.16E−02 | 1.05E−07 |
| 612 | 1.13E+05 | 1.22E−02 | 1.08E−07 |
| CEA-BsAb | 3.68E+03 | 2.70E−02 | 7.36E−06 |
| HER2-BsAb | 1.28E+05 | 1.26E−02 | 9.84E−08 |

TABLE 21
Affinity of unilateral CD3/CEA-CAM6/EGFR trispecific
antibodies to CD3ε/δ (T = 30° C.)
Antigen: CD3ε/δ

| Sample | ka (1 M⁻¹s⁻¹) | kdis (1/s) | KD (M) |
|---|---|---|---|
| 601 | 3.79E+05 | 3.78E−04 | 9.99E−10 |
| 603 | 1.73E+05 | 1.05E−04 | 6.08E−10 |
| 609 | 2.12E+05 | 1.97E−04 | 9.27E−10 |
| 610 | 2.73E+05 | 4.02E−04 | 1.47E−09 |
| 611 | 4.10E+05 | 7.27E−04 | 1.77E−09 |
| 612 | 3.86E+05 | 6.82E−04 | 1.77E−09 |
| CEA-BsAb | 2.75E+05 | 4.50E−04 | 1.64E−09 |
| HER2-BsAb | 2.56E+05 | 3.86E−04 | 1.51E−09 |

The experimental results showed that the unilateral CD3/CEA-CAM6/HER2 trispecific antibodies had an affinity of 4.16 nM-13.3 nM with CEA-CAM6, an affinity of 15.4 nM-38.9 nM with HER2, and an affinity of 0.351 nM-1.80 nM with CD3. The KD data for binding to different antigens are shown in the tables below.

TABLE 22
Affinity of unilateral CD3/CEA-CAM6/EGFR trispecific
antibodies to CEA-CAM6 (T = 30° C.)
Antigen: CEA-CAM6

| Sample | ka (1 M⁻¹s⁻¹) | kdis (1/s) | KD (M) |
|---|---|---|---|
| 701 | 2.23E+05 | 2.97E−03 | 1.33E−08 |
| 703 | 3.63E+05 | 1.87E−03 | 5.16E−09 |
| 709 | 4.33E+05 | 1.80E−03 | 4.16E−09 |
| 710 | 3.98E+05 | 1.79E−03 | 4.48E−09 |
| 711 | 5.62E+05 | 2.54E−03 | 4.52E−09 |
| 712 | 4.22E+05 | 2.23E−03 | 5.30E−09 |

TABLE 23
Affinity of unilateral CD3/CEA-CAM6/EGFR trispecific
antibodies to HER2 (T = 30° C.)
Antigen: HER2

| Sample | ka (1 M⁻¹s⁻¹) | kdis (1/s) | KD (M) |
|---|---|---|---|
| 701 | 1.85E+05 | 6.93E−03 | 3.75E−08 |
| 703 | 2.18E+05 | 3.56E−03 | 1.63E−08 |
| 709 | 2.19E+05 | 4.55E−03 | 2.07E−08 |
| 710 | 2.49E+05 | 3.83E−03 | 1.54E−08 |
| 711 | 2.28E+05 | 8.88E−03 | 3.89E−08 |
| 712 | 2.30E+05 | 5.01E−03 | 2.18E−08 |

TABLE 24
Affinity of unilateral CD3/CEA-CAM6/EGFR trispecific
antibodies to CD3ε/δ (T = 30° C.)
Antigen: CD3ε/δ

| Sample | ka (1 M⁻¹s⁻¹) | kdis (1/s) | KD (M) |
|---|---|---|---|
| 701 | 3.09E+05 | 4.02E−04 | 1.30E−09 |
| 703 | 2.22E+05 | 8.40E−05 | 3.79E−10 |
| 709 | 1.76E+05 | 6.18E−05 | 3.51E−10 |
| 710 | 1.86E+05 | 1.27E−04 | 6.86E−10 |
| 711 | 3.67E+05 | 5.16E−04 | 1.41E−09 |
| 712 | 3.88E+05 | 6.98E−04 | 1.80E−09 |

### Example 13: Unilateral CD3/CEA-CAM6/EGFR, CD3/CEA/HER2 and CD3/CEA-CAM6/HER2 Trispecific Antibodies Induce T Cell Mediated Killing of Tumor Cells

T cells mediated killing induced by unilateral CD3/CEA-CAM6/EGFR tri-specific antibodies against target cells were assessed in human tumor cells NCI-H157-21# (high expression levels of CEA-CAM6, low expression levels of EGFR), MDA-MB-453 (low expression levels of CEA-CAM6, EGFR negative), SK-OV-3 (high expression levels of CEA-CAM6, medium expression levels of EGFR), NCI-H1573 (high expression levels of CEA-CAM6, high expression levels of EGFR), SK-BR-3 (low expression levels of CEA-CAM6, low expression levels of EGFR), NCI-H1975 (high expression levels of CEA-CAM6, medium expression levels of EGFR), MDA-MB-468 (high expression levels of CEA-CAM6, medium expression levels of EGFR) and MKN-45 (high expression levels of CEA-CAM6, medium expression levels of EGFR).

T cells mediated killing induced by unilateral CD3/CEA/HER2 tri-specific antibodies against target cells were assessed in human tumor cells MDA-MB-453 (medium expression levels of CEA, medium expression levels of HER2), SK-OV-3 (CEA negative, high expression levels of HER2), NCI-H1573 (high expression levels of CEA, HER2 negative), MCF-7 (low expression levels of CEA, low expression levels of HER2), SK-BR-3 (low expression levels of CEA, medium expression levels of HER2) and MKN-45 (high expression levels of CEA, HER2 negative).

T cells mediated killing induced by unilateral CD3/CEA-CAM6/HER2 tri-specific antibodies against target cells were assessed in human tumor cells MDA-MB-453 (medium expression levels of CEA-CAM6, medium expression levels of HER2), SK-OV-3 (high expression levels of CEA-CAM6, high expression levels of HER2), NCI-H1573 (high expression levels of CEA-CAM6, HER2 negative), MCF-7 (low expression levels of CEA-CAM6, low expression levels of HER2), SK-BR-3 (low expression levels of CEA-CAM6, medium expression levels of HER2) and MKN-45 (high expression levels of CEA-CAM6, HER2 negative).

Human PBMCs were used as effectors and killing was detected after 72 h incubation with antibodies.

Briefly, tumor cells were digested with trypsin/EDTA, washed once with pre-chilled PBS, re-suspended in RPMI 1640 medium containing 10% FBS, and plated at a density of 5,000 cells/well in a flat-bottom 96-well plate (Corning 3599). After culturing for 4 h, 50 μL of serially diluted antibody solution was added to each well. Each concentration was added to three replicate wells. The antibodies were incubated with the tumor cells for more than 30 min to fully attach the proteins to the cells. PBMC cells (Reid Bio, 1521) were thawed quickly, resuscitated, added to 10 mL of RPMI 1640 medium containing 10% FBS, centrifuged at 1000 rpm for 5 min, and re-suspended after discarding the supernatant. Cell density was adjusted and 50 μL of PBMCs was added to each well of target cells to reach a final E:T ratio of 10:1. The 96-well plate was placed in a 37° C., 5% CO₂ incubator for 3 days.

After culturing for 3 days, the cell culture supernatant was carefully removed. The plate was pat dried with a paper towel. Serum-Free DMEM containing 10% CCK8 detection solution (Dojindo, CK04-20) was added to the 96-well plate and incubated at 37° C. for 2-4 hours. After 4 hours, the absorbance at 450 nm was detected using a microplate reader (BioTech, Synergy LX). The cell viability was calculated according to the below formula:

$$cell viability = [ ( A s - Ab ) / ( Ac ‐ A b ) ] \times 100 \% .$$

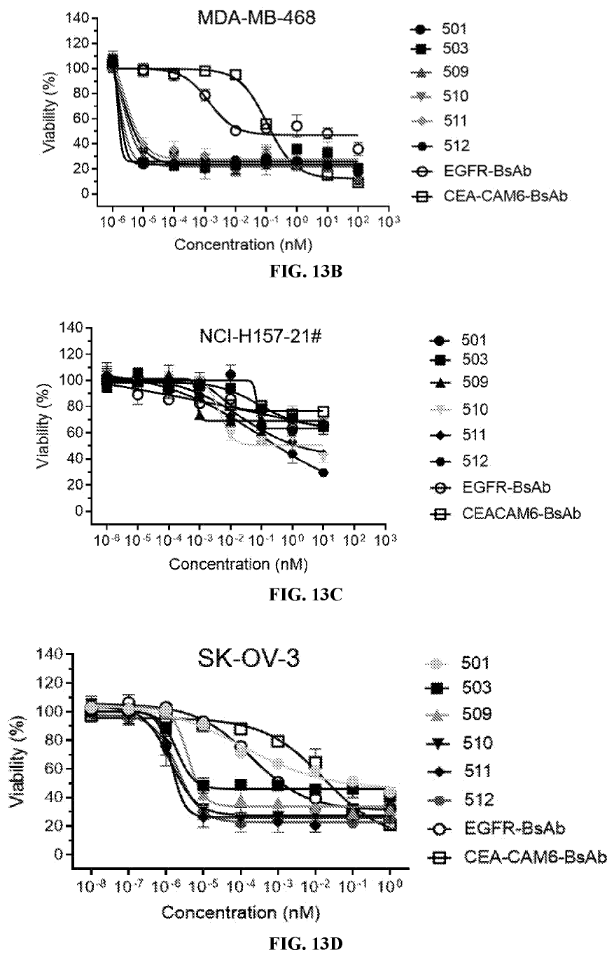

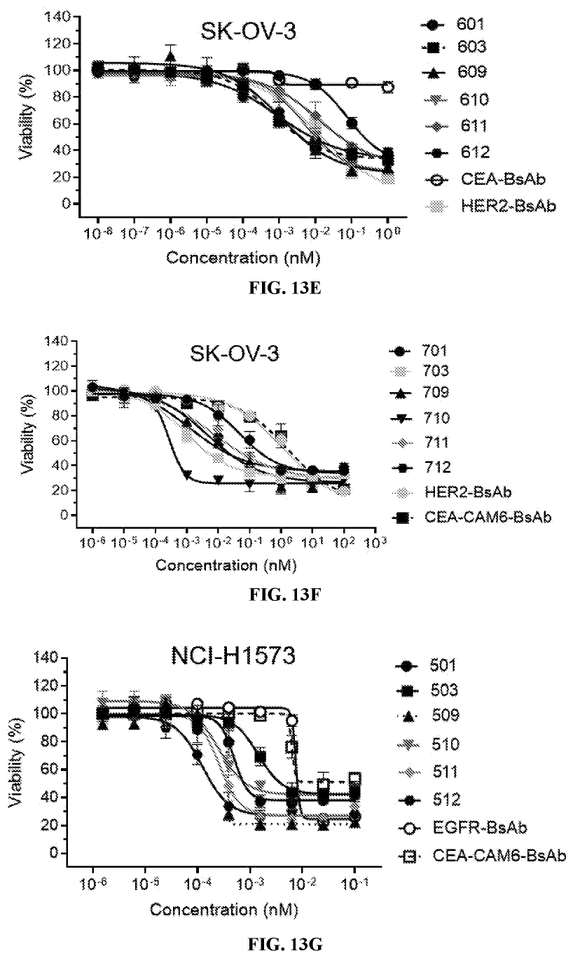

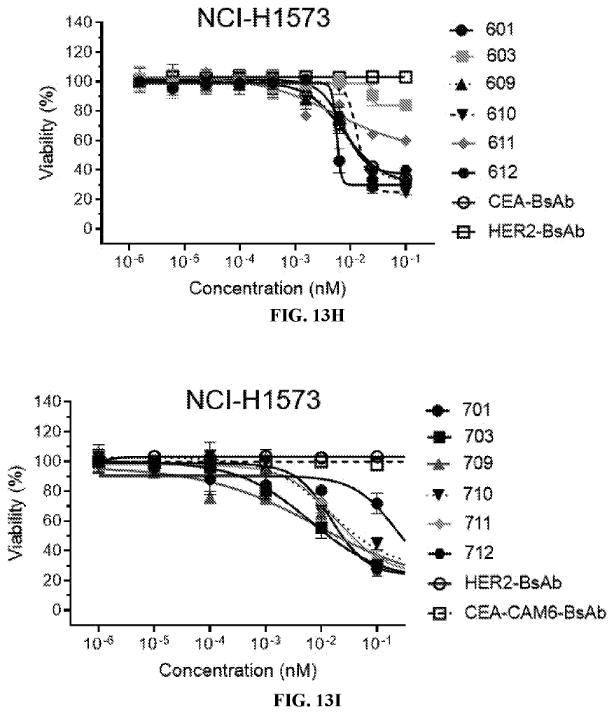

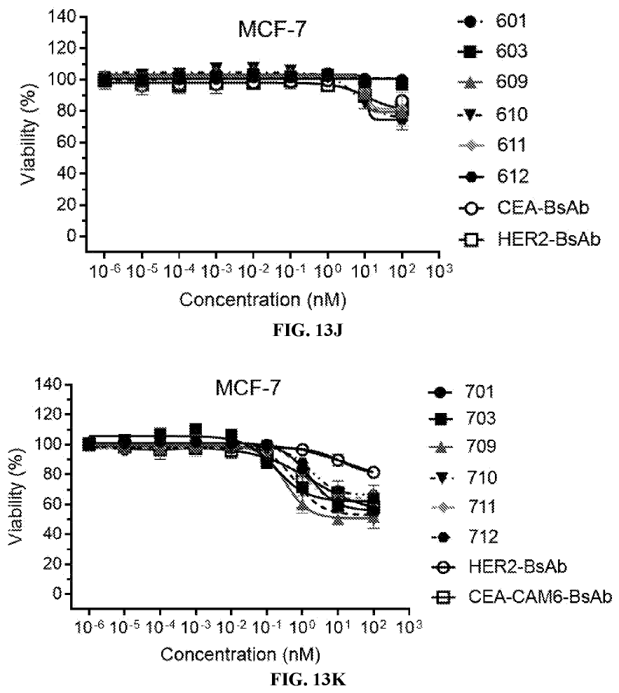

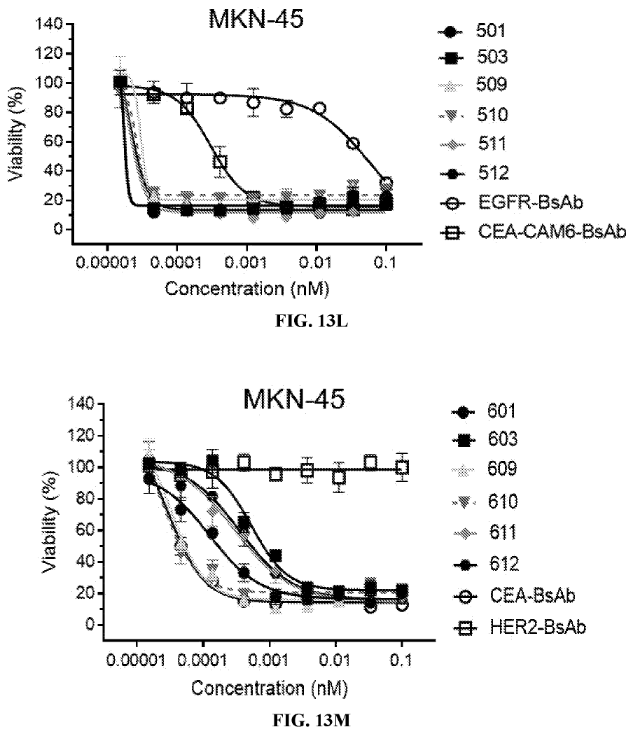

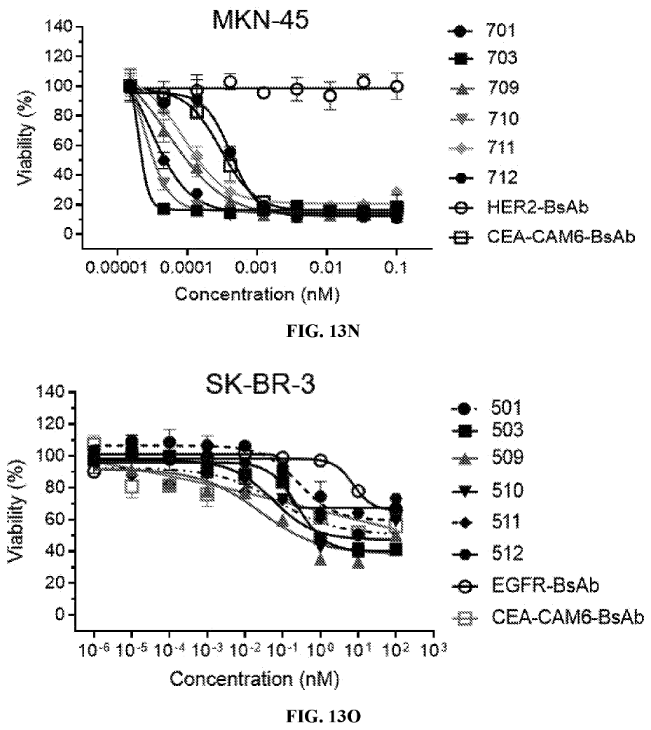

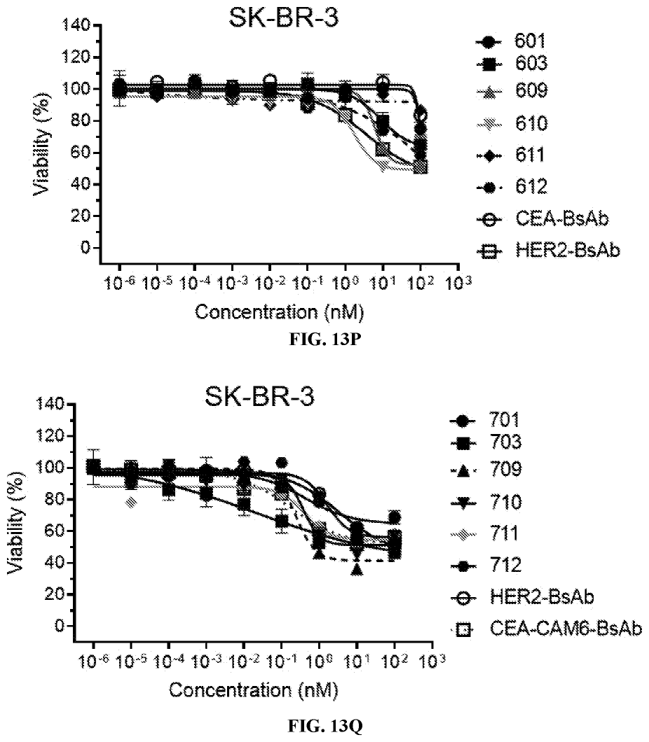

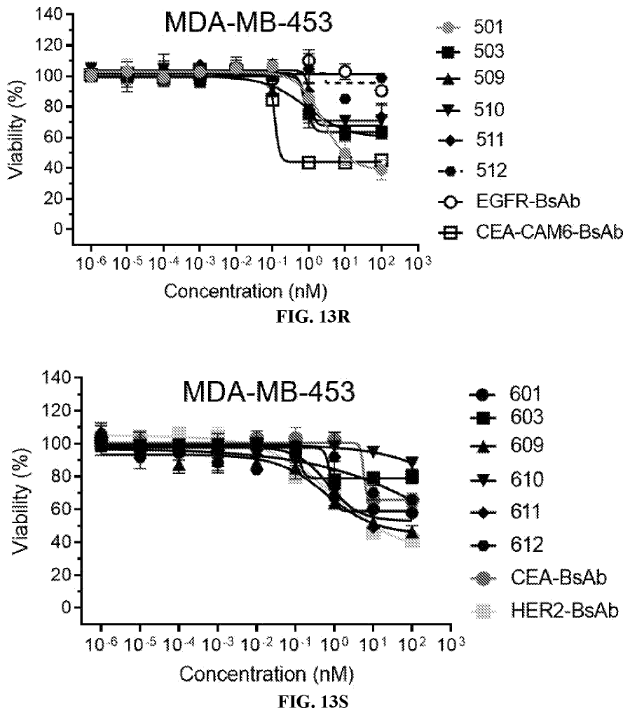

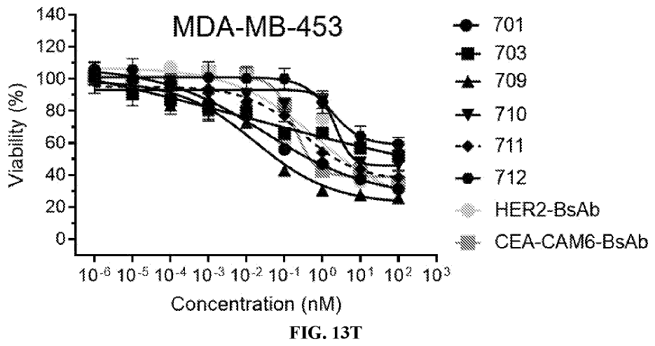

The results showed that the unilateral trispecific antibodies induced PBMC to kill the tumor antigen positive cells (FIG. 13). EC50 values related to cell killing ability calculated using GraphPad Prism are shown in the below tables.

For cell lines with high expression of CEA-CAM6 or EGFR (SK-OV-3, NCI-H1573, MDA-MB-468 and MKN-45), the unilateral CD3/CEA-CAM6/EGFR tri-specific antibodies had significantly stronger cell killing effects than the bispecific control antibodies (anti-EGFR-BsAb and CEA-CAM6-BsAb).

In NCI-H1975, a cell line with high expression of CEA-CAM6 and EGFR, the cell killing effects of the unilateral trispecific antibodies were significantly stronger than that of the bispecific control antibody anti-CEA-CAM6-BsAb, and were close to (511, 512) or stronger than (501, 503, 509, 510) that of the bispecific control antibody anti-EGFR-BsAb.

In NCI-H157-21#, a cell line with low expression of EGFR and high expression of CEA-CAM6, the killing effects of unilateral trispecific antibodies were stronger than that of EGFR-BsAb, and the killing effects of 509 and 510 were stronger than that of CEA-CAM6-BsAb.

In MDA-MB-453, a cell line with low expression of EGFR and high expression of CEA-CAM6, the unilateral trispecific antibodies showed stronger killing effects than EGFR-BsAb, and some unilateral trispecific antibodies (503, 509, 510) have similar killing effects comparing to CEA-CAM6-BsAb.

In SK-BR-3, a cell line with low expression of EGFR and CEA-CAM6, unilateral trispecific antibodies showed stronger killing effects than EGFR-BsAb and CEA-CAM6-BsAb.

The above results show that the unilateral CD3/CEA-CAM6/EGFR trispecific antibodies had significant killing effects on cell lines with different expressions of CEA-CAM6/EGFR. The results are shown in FIG. 13 and the table below.

TABLE 25
Killing effects of unilateral CD3/CEA-CAM6/EGFR trispecific antibodies on tumor cells (EC50: nM)

| Sample | NCI-H157-21# | MDA-MB-453 | SK-OV-3 | NCI-H1573 | SK-BR-3 | NCI-H1975 | MDA-MB-468 | MKN-45 |
|---|---|---|---|---|---|---|---|---|
| 501 | 7.12E−02 | 2.498 | 2.47E−04 | 4.94E−04 | 0.2661 | 1.03E−04 | 1.65E−06 | 1.72E−05 |
| 503 | 8.10E−02 | 0.8603 | 3.31E−05 | 1.42E−03 | 0.2499 | 8.68E−04 | 1.46E−06 | 1.74E−05 |
| 509 | 7.90E−04 | 0.8595 | 1.71E−05 | 3.36E−04 | 0.02512 | 4.37E−04 | 1.96E−06 | 2.98E−05 |
| 510 | 4.53E−03 | 0.717 | 9.69E−06 | 2.44E−04 | 0.05413 | 4.19E−03 | 1.57E−06 | 2.61E−05 |
| 511 | 2.16E−02 | 1.084 | 2.27E−05 | 2.57E−04 | 0.07084 | 1.20E−02 | 2.40E−06 | 2.02E−05 |
| 512 | 9.00E−02 | 2.876 | 9.02E−06 | 1.22E−04 | 0.1234 | 1.95E−02 | 2.08E−06 | 2.09E−05 |
| EGFR- | 7.516 | >100 | 1.10E−03 | 7.48E−03 | 7.399 | 5.57E−03 | 1.34E−03 | 5.72E−02 |
| BsAb | | | | | | | | |
| CEA- | 4.65E−03 | 0.1145 | 1.14E−02 | 6.28E−03 | 4.195 | 5.63E−02 | 0.1082 | 3.04E−04 |
| CAM6- | | | | | | | | |
| BsAb | | | | | | | | |

In MDA-MB-453, a cell line with moderate expression of CEA and HER2, except for 610 and 612, unilateral CD3/CEA/HER2 trispecific antibodies showed stronger killing effects comparing to CEA-BsAb and HER2-BsAb.

In SK-OV-3, a CEA-negative cell line with high HER2 expression, the unilateral CD3/CEA/HER2 trispecific antibodies showed stronger killing effects than CEA-BsAb, and some antibodies showed similar (601, 603, 609, 610, 611) or stronger (612) killing effects comparing to HER2-BsAbs.

In MCF-7, a cell line with low expression of CEA and HER2, the unilateral trispecific antibodies (609, 610, 611, 612) showed stronger killing effects than CEA-BsAb and HER2-BsAb.

In SK-BR-3, a cell line with low expression of CEA and medium expression of HER2, the killing effects of unilateral trispecific antibodies (603, 609, 610) is stronger than that of CEA-BsAb and HER2-BsAb.

In NCI-H1573, a HER2-negative cell line with high expression of CEA, the unilateral CD3/CEA/HER2 trispecific antibodies showed stronger killing effects than HER2-BsAb, similar (603, 609, 610) or stronger (603, 609, 610) 601, 611, 612) killing effects comparing to CEA-BsAb.

In MKN-45, a HER2-negative cell line with high expression of CEA, the unilateral CD3/CEA/HER2 trispecific antibodies showed stronger killing effects than HER2-BsAb and HER2-BsAb.

The results showed that the six unilateral CD3/CEA/HER2 trispecific antibodies had significant killing effects on cell lines with different expressions of CEA/HER2. The results are shown in FIG. 13 and the table below.

TABLE 26
Killing effects of unilateral CD3/CEA/HER2 trispecific
antibodies on tumor cells (EC50: nM)

| Sample | MDA-MB-453 | SK-OV-3 | NCI-H1573 | MCF-7 | SK-BR-3 | MKN-45 |
|---|---|---|---|---|---|---|
| 601 | 0.816 | 1.26E−03 | 5.60E−03 | >100 | >100 | 1.25E−04 |
| 603 | 0.1305 | 1.01E−02 | 2.48E−02 | >100 | 8.447 | 5.34E−04 |
| 609 | 0.6294 | 6.66E−04 | 8.40E−03 | 10.15 | 6.031 | 3.23E−05 |
| 610 | >100 | 3.26E−03 | 1.36E−02 | 5.97 | 1.709 | 2.52E−05 |
| 611 | 0.667 | 1.36E−03 | 5.23E−03 | 10.58 | >100 | 2.94E−04 |
| 612 | >100 | 9.66E−05 | 7.45E−03 | 10.94 | >100 | 3.53E−04 |
| CEA-BsAb | 5.565 | >100 | 7.55E−03 | >100 | 80.76 | 5.72E−02 |
| HER2-BsAb | 0.9762 | 1.97E−04 | >100 | 11.43 | 3.5 | >100 |

In MDA-MB-453, a cell line with moderate expression of CEA-CAM6 and HER2, the unilateral CD3/CEA-CAM6/HER2 trispecific antibodies showed a stronger (701, 703, 709) or similar (710, 711, 712) killing effects comparing to CEA-BsAb and HER2-BsAb.

In SK-OV-3, a cell line with high expression of CEA-CAM6 and HER2, the unilateral CD3/CEA-CAM6/HER2 trispecific antibodies showed stronger killing effects than CEA-BsAb and HER2-BsAb.

In NCI-H1573 and MKN-45, HER2-negative cell lines with high expression of CEA-CAM6, the unilateral CD3/CEA-CAM6/HER2 trispecific antibodies showed stronger cell killing effects than HER2-BsAb. In NCI-H1573, unilateral trispecific antibodies (703, 709, 710, 711, 712) showed similar killing effects than that of CEA-CAM6-BsAb. In MKN-45, the killing effects of the unilateral trispecific antibodies were similar to (701) or stronger than (703, 709, 710, 711, 712) CEA-CAM6-BsAb.

In MCF-7 and SK-BR-3, cell lines with low expression of CEA-CAM6 and low/medium expression of HER2, the unilateral CD3/CEA-CAM6/HER2 trispecific antibodies showed killing effects that are similar to or stronger than the killing effects of CEA-BsAb and HER2-BsAb.

The results showed that the six unilateral CD3/CEA-CAM6/HER2 trispecific antibodies had significant killing effects on cell lines with different expressions of CEA-CAM6/HER2. The results are shown in FIG. 13 and the table below.

TABLE 27
Killing effects of unilateral CD3/CEA-CAM6/HER2
tri-specific antibodies on tumor cells (EC50: nM)

| Sample | MDA-MB-453 | SK-OV-3 | NCI-H1573 | MCF-7 | SK-BR-3 | MKN-45 |
|---|---|---|---|---|---|---|
| 701 | 0.03008 | 1.04E−05 | 0.2242 | 1.563 | 0.4194 | 4.24E−04 |
| 703 | 0.0107 | 2.52E−06 | 6.23E−03 | 0.1775 | 0.02131 | 2.02E−05 |
| 709 | 0.01753 | 6.32E−06 | 8.79E−03 | 0.3228 | 0.2317 | 5.34E−05 |
| 710 | 2.288 | 3.06E−06 | 1.49E−02 | 0.5877 | 0.3838 | 2.33E−05 |
| 711 | 0.2932 | 6.21E−06 | 1.84E−02 | 0.9959 | 0.4131 | 8.99E−05 |
| 712 | 1.677 | 6.98E−06 | 1.50E−02 | 1.858 | 1.634 | 2.88E−05 |
| HER2-BsAb | 0.669 | 1.98E−04 | >100 | 11.43 | 3.5 | >100 |
| CEA- | 0.183 | 1.14E−02 | 6.28E−03 | 1.798 | 4.195 | 3.04E−04 |
| CAM6- | | | | | | |
| BsAb | | | | | | |

### Example 14: Thermal Stability of Trispecific Antibody Detected by DSF Method

Differential scanning fluorimetry (DSF) is a method of slowly heating a sample on a fluorescent quantitative PCR machine, and detecting the amount of fluorescent dye combined with a protein whose structure changes during the heating process, to evaluate the thermal stability of the protein.

The thermal stability of the trispecific antibodies was monitored by DSF. After mixing 19 μl of 5 μM protein sample with 1 μl 200× sypro orange (ThermoFisher, 56650) thoroughly, the mixture was added to a 96-well plate (Applied Biosystems, N8010560) in triplicates. After incubating at 25° C. for 30 s, the temperature was increased from 25° C. to 95° C. at a rate of 0.05° C./min, and the fluorescence signal intensity was collected by a real-time fluorescent quantitative PCR instrument (Applied Biosystems, QuantStudio 5 System). PBS was used as a blank control. After the experiment, the Tm value was analyzed using Protein Thermal Shift software.

Tm was measured three times from triplicate wells. The experimental results are shown in the table below. The Tm1 value of the unilateral trispecific antibodies was 59.4° C.-63.2° C. The Tm2 value of the unilateral trispecific antibodies was 69.5° C.-74.5° C.

TABLE 28
Tm value of unilateral trispecific antibodies
detected by the DSF method

| | Average | Average |
|---|---|---|
| Sample | Tm1 (° C.) | Tm2 (° C.) |
| 401 | 62.7 | 72.9 |
| 402 | 61.9 | 74.0 |
| 403 | 61.9 | 72.6 |
| 405 | 61.5 | 72.8 |
| 406 | 61.6 | 72.8 |
| 407 | 61.7 | 72.4 |
| 408 | 61.7 | 72.2 |
| 409 | 59.5 | 72.4 |
| 410 | 59.4 | 72.9 |
| 411 | 59.7 | 72.4 |
| 412 | 59.8 | 72.8 |
| 413 | 61.4 | 72.2 |
| 414 | 61.7 | 72.5 |
| 415 | 61.1 | 72.3 |
| 416 | 61.6 | 72.0 |
| 501 | 61.6 | 71.9 |
| 503 | 61.6 | 72.1 |
| 509 | 59.7 | 70.9 |
| 510 | 60.6 | 73.9 |
| 511 | 59.9 | 72.3 |
| 512 | 60.2 | 73.1 |
| 601 | 60.9 | 72.3 |
| 603 | 61.4 | 72.4 |
| 609 | 62.5 | 70.5 |
| 610 | 62.5 | 70.5 |
| 611 | 61.9 | 69.8 |
| 612 | 61.8 | 71.1 |
| 701 | 62.6 | 72.1 |
| 703 | 60.4 | 72.1 |
| 709 | 61.0 | 72.0 |
| 710 | 62.1 | 70.5 |
| 711 | 61.7 | 69.5 |
| 712 | 61.5 | 70.0 |
| CEA-BsAb | 63.2 | 72.7 |
| EGFR-BsAb | 62.2 | 74.5 |
| HER2-BsAb | 62.4 | 72.5 |
| CEA-CAM6-BsAb | 62.8 | 72.3 |

### Example 15: Detection of Multispecificity of Unilateral Trispecific Antibodies by ELISA

The experimental steps are as follows
(1) Antigen coating: cardiolipin (Sigma, cat. C0563), keyhole limpet haemocyanin (KLH, Sigma, H8283), LPS (Sigma, L6529), ssDNA (Sigma, D8899), dsDNA (Sigma, D4522) and Insulin (abs42019847) were plated at 50 μg/mL, 5 μg/mL, 10 μg/mL, 1 μg/mL, 1 μg/mL, and 5 μg/mL per well in a 96-well plate (Thermo Nunc, 475094), and incubated at 4° C. overnight;
(2) The next morning, the plate was washed three times with 0.05% PBST (Biotek, 4052S Microplate washer), blocked in PBS solution containing 3% skim milk for 1 h at room temperature, and washed three times with 0.05% PBST;
(3) 50 μl of 100 nM antibodies or blank control PBS (3 replicate wells for each sample) were added to each well and incubated for 1 h at room temperature; the plate was washed 3 times with 0.05% PBST;
(4) 50 μl anti-human IgG-HRP conjugate (Sigma, AP113P, 1:8000 dilution) was added to each well; the plate was incubated for 1 h at room temperature and washed 6 times with 0.05% PBST;
(5) 50 μl of TMB chromogenic solution (Biopanda, TMB-S-001) was added to each well and incubated at room temperature for 10-15 minutes for color development;
(6) 50 μl of 1M HCl was added to each well to stop the color development, and the absorbance at 450 nm was measured using a microplate reader (Biotek, SYNERGY LX); Data analysis: If the ratio between the absorbance of the antibody and the absorbance of the blank control PBS is ≥3, there is too much non-specific binding.

The experimental results are shown in the table below. From the results, it can be seen that none of the tested trispecific antibodies showed too much non-specific binding, indicating that these trispecific antibodies do not have non-specific binding to the six tested antigens.

TABLE 29
Absorbance ratios of trispecific antibodies bound to various substances

| Sample | Cardiolipin | KLH | LPS | SSDNA | dsDNA | Insulin |
|---|---|---|---|---|---|---|
| 401 | 1.59 | 0.88 | 0.67 | 1.23 | 1.09 | 1.12 |
| 402 | 1.67 | 1.00 | 0.65 | 2.50 | 1.94 | 1.20 |
| 403 | 1.33 | 1.32 | 0.95 | 2.23 | 2.06 | 2.70 |
| 405 | 2.48 | 1.52 | 1.16 | 1.97 | 1.96 | 1.43 |
| 406 | 2.08 | 0.94 | 0.72 | 1.38 | 1.49 | 1.53 |
| 407 | 1.55 | 1.12 | 1.16 | 2.89 | 2.77 | 1.35 |
| 408 | 1.43 | 1.06 | 1.10 | 2.35 | 2.61 | 1.44 |
| 409 | 1.59 | 1.01 | 1.04 | 2.16 | 2.63 | 1.46 |
| 410 | 1.56 | 1.00 | 1.04 | 2.76 | 2.50 | 1.27 |
| 411 | 1.42 | 0.98 | 1.01 | 2.86 | 2.53 | 1.19 |
| 412 | 1.43 | 0.90 | 1.00 | 1.20 | 1.04 | 1.30 |
| 413 | 1.86 | 1.73 | 1.79 | 2.08 | 2.49 | 1.23 |
| 414 | 1.50 | 1.32 | 1.25 | 2.22 | 2.48 | 1.16 |
| 415 | 1.46 | 1.15 | 1.07 | 1.55 | 1.14 | 1.10 |
| 416 | 1.47 | 1.12 | 1.07 | 1.99 | 2.41 | 1.16 |
| 501 | 1.86 | 0.65 | 0.80 | 2.90 | 2.86 | 0.57 |
| 503 | 2.46 | 2.29 | 2.60 | 1.04 | 2.30 | 1.48 |
| 509 | 1.21 | 0.60 | 0.53 | 2.22 | 2.78 | 0.45 |
| 510 | 2.13 | 2.84 | 2.73 | 1.18 | 2.45 | 1.50 |
| 511 | 1.67 | 0.80 | 0.58 | 1.93 | 1.98 | 0.83 |
| 512 | 2.90 | 2.68 | 2.86 | 1.11 | 2.72 | 1.69 |
| 601 | 0.85 | 0.50 | 0.41 | 2.66 | 2.09 | 0.66 |
| 603 | 2.00 | 2.68 | 2.81 | 2.10 | 1.68 | 2.08 |
| 609 | 1.23 | 0.86 | 0.82 | 1.81 | 1.19 | 1.17 |
| 610 | 2.16 | 2.60 | 2.29 | 2.42 | 1.59 | 2.01 |
| 611 | 0.73 | 0.56 | 0.37 | 1.79 | 1.40 | 1.09 |
| 612 | 2.24 | 2.40 | 2.11 | 2.08 | 1.29 | 1.37 |
| 701 | 0.77 | 0.69 | 0.40 | 2.59 | 2.99 | 1.27 |
| 703 | 1.60 | 1.06 | 1.68 | 2.29 | 1.40 | 1.16 |
| 709 | 0.54 | 0.64 | 0.36 | 1.71 | 1.70 | 1.03 |
| 710 | 1.92 | 2.26 | 2.57 | 2.04 | 1.30 | 1.58 |
| 711 | 0.50 | 0.75 | 0.32 | 1.04 | 1.06 | 0.67 |
| 712 | 2.66 | 2.75 | 1.62 | 1.25 | 2.69 | 2.65 |
| CEA-BsAb | 1.53 | 2.16 | 0.48 | 2.18 | 2.55 | 1.24 |
| EGFR-BsAb | 0.81 | 0.50 | 2.04 | 0.25 | 0.16 | 0.86 |
| HER2-BsAb | 2.82 | 2.31 | 0.58 | 1.97 | 2.40 | 1.69 |
| CEA-CAM6-BsAb | 0.63 | 0.45 | 2.22 | 0.19 | 0.08 | 0.87 |
| Ofatumumab | 1.14 | 1.15 | 1.01 | 1.02 | 1.06 | 1.02 |
| Adalimumab | 0.98 | 1.03 | 0.99 | 0.98 | 0.97 | 1.23 |

### Example 16: Detection of Self-Interaction of Trispecific Antibodies by Rio-Layer Interferometry (BLI) Assay

Biofilm interferometry (BLI) experiments were performed on OCTECT RED96e (ForteBio) at 30° C. using PBS (10 mmol/L Na₂HPO₄, 1.75 mmol/L KH₂PO₄, 137 mmol/L NaCl, 2.65 mmol/L KCl, pH 7.2-7.4) as the running buffer.

Trispecific antibodies and control antibodies (e.g., Ofatumumab) were first captured on the surface of an AHQ sensor (Anti-hIgG Fc, sartorius, 18-5001) chip with immobilized anti-human Fe antibodies. The bispecific control antibodies were diluted to 1 μM, and the proteins were coupled to the surface of the AHQ sensor chip in PBS, and the signal value was ˜0.8 nm. Subsequently, the sites on the AHQ sensor that were not bound to the test antibodies were fully blocked with human IgG antibody, and the self-binding signal of the trispecific antibodies or the control antibodies was analyzed. The experimental parameters are as follows: Baseline1: 60 s, Loading: 180 s, Loading response: 0.8 nm, Baseline2: 180 s, Association: 240 s, High sensitivity kinetics: 2 Hz. Binding curves were obtained using the Date Analysis HT 12 software (sartorius, 50-5029). If the self-binding signal of the test antibody is higher than that of the control antibody by more than 0.1 nm, the test antibody is considered to have self-interaction; otherwise, it is considered to have no self-interaction. The experimental results are shown in the table below.

The positive control Ofatumumab self-interaction signal is 0.11 nm, and the trispecific antibodies self-interaction signal is in the range of 0.037 nm-0.121 nm, which is below 0.21 nm (0.11 nm+0.1 nm), signaling weak self-interaction and suggesting that the trispecific antibodies have good solubility.

TABLE 30
Self-interacting signals of trispecific antibodies as detected by BLI

| Sample | BLI-Response(nm) |
|---|---|
| 401 | 0.127 |
| 403 | 0.092 |
| 409 | 0.106 |
| 410 | 0.121 |
| 411 | 0.101 |
| 412 | 0.089 |
| 501 | 0.104 |
| 503 | 0.072 |
| 509 | 0.092 |
| 510 | 0.137 |
| 511 | 0.084 |
| 512 | 0.07 |
| 601 | 0.096 |
| 603 | 0.084 |
| 609 | 0.098 |
| 610 | 0.088 |
| 611 | 0.07 |
| 612 | 0.082 |
| 701 | 0.094 |
| 703 | 0.108 |
| 709 | 0.083 |
| 710 | 0.09 |
| 711 | 0.104 |
| 712 | 0.073 |
| CEA-BsAb | 0.103 |
| EGFR-BsAb | 0.037 |
| HER2-BsAb | 0.066 |
| CEA-CAM6-BsAb | 0.045 |
| Ofatumumab | 0.11 |

### Example 17: Construction and Preparation of CD3/c-MET/EGFR Trispecific Antibody

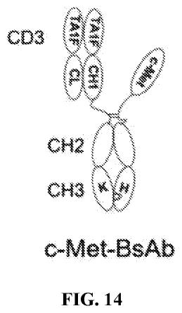

The c-Met single-domain antibody and EGFR single-domain antibody (VHH) were obtained by immunizing llamas (lama). These are abbreviated as c-Met and EGFR in the structural schematics. Based on the methods in Example 6, based on the structures of 401-409 and 413-415, we designed CD3/c-Met/EGFR trispecific antibodies, which were named 801-809 and 813-815, respectively (see FIGS. 15A-15L for details). At the same time, bispecific control antibodies (c-Met BsAb and EGFR BsAb) containing anti-CD3 fab TA1F were designed (see FIG. 14). TA1F represents the Fab region of the anti-CD3 antibody clone TAL. Anti-CD3 VH was linked to CH1-Hinge-Fc (IgG4), and anti-CD3 VL was linked to CL (kappa). Table 1 lists the common sequences. Table 2 lists the sequence of the EGFR single-domain antibody. The table below lists the sequence of the c-Met single-domain antibody.

TABLE 31
Sequence Listing

| Name | SEQ ID NO. | Sequence |
|---|---|---|
| c-Met VHH | 13 | QVKLEESGGDLVQPGGSLRLSCVGSGSIFSYTAMGWYRQAPGKQRELVATIHSGGNINY |
| | | ADSVKGRFTISRDNTENTVYLQMNSLKPEDTAVYYCWSLLVGPETRYKDFNFWGRGTQ |
| | | VTVSS |

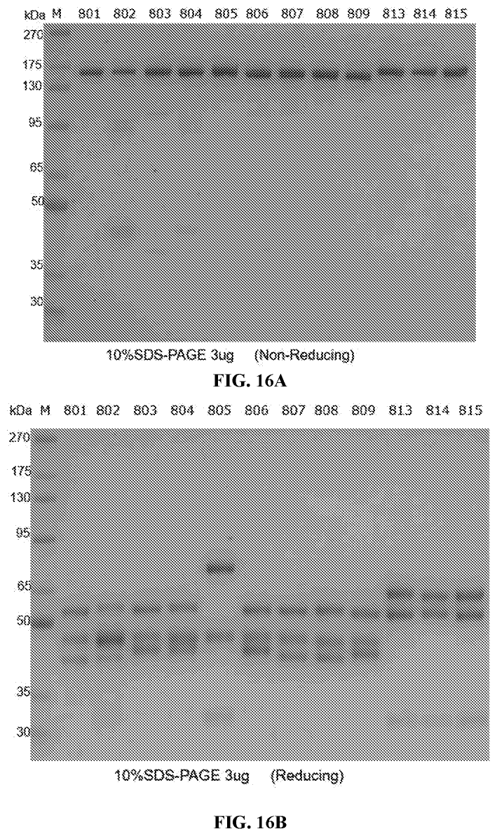

Based on the methods in Examples 2 and 7, the above plasmids were transfected into Expi 293F™ cells through PEI, and various purification methods were used to prepare antibodies. The results of protein SDS-PAGE are shown in FIGS. 16A-16B. The output of the tertiary antibody was 12.7-45.92 mg/L, and the purity of the tertiary antibody was ≥90%. The table below shows the yield and purity data.

TABLE 32
Yield and purity of CD3/C-MET/EGFR trispecific antibody

| Sample | Yield(mg/L) | Purity (%) |
|---|---|---|
| 801 | 39.24 | 92 |
| 802 | 45.92 | 90 |
| 803 | 17.7 | 90 |
| 804 | 52.5 | 90 |
| 805 | 21.8 | 92 |
| 806 | 25.6 | 90 |
| 807 | 12.7 | 92 |
| 808 | 16.3 | 92 |
| 809 | 13.9 | 90 |
| 813 | 31.4 | 92 |
| 814 | 15.0 | 92 |
| 815 | 34.9 | 92 |

### Example 18: Determination of the Affinity of CD3/C-MET/EGFR Trispecific Antibody by Bio-Layer Interferometry (BLI)

Biofilm interferometry (BLI) experiments were performed on OCTECT RED96e (ForteBio) at 30° C. using 0.02% PBST (10 mmol/L Na₂HPO₄, 1.75 mmol/L KH₂PO₄, 137 mmol/L NaCl, 2.65 mmol/L KCl, pH 7.2-7.4, 0.02% surfactant Tween 20) as the running buffer.

Antibodies were first captured onto an anti-human IgG Fc coated AHC biosensor (Anti-hIgG Fc, Sartorius, 18-5060). Antibodies were diluted to 5 μg/mL and 1.5 nm protein was coupled to the AHC sensor surface in 0.02% PBST.

The analytes, including (1) recombinant human CD3ε/δ protein (ACRO systems, CDD-H52W1), (2) recombinant human c-Met protein (ACRO systems, MET-H5227), and (3) recombinant human EGFR protein (ACRO systems, EGR-H5222), were diluted in 0.02% PBST to 200 nM, 100 nM, 50 nM, 25 nM, 12.5 nM, 6.25 nM, and 3.125 nM.

After the sensor captures the antibodies, it is used to bind and dissociate with different concentrations of analytes to obtain the KD value of the interaction. The experimental parameters are as follows: Baseline1: 60 s, Loading: 240 s, Baseline2: 180 s, Association: 240 s, Dissociation: 600 s, High sensitivity kinetics: 2 Hz. Kinetic constants were derived based on the rate equation for 1:1 Langmuir binding to obtain equilibrium dissociation constants (KD). The Data Analysis HT 12 software (Sartorius, 50-5029) was used to simultaneously fit binding and dissociation curves to calculate Kₒₙ and K_{off}. The affinity KD was calculated based on the below formula: KD=K_{off}/Kₒₙ.

The experimental results showed that the affinity of the unilateral trispecific antibodies to human CD3 was 0.17 nM-3.14 nM, the affinity to EGFR was 0.6 nM-17 nM, and the affinity to c-Met was 0.47 nM-1.93 nM. The KD between trispecific antibodies and different antigens are shown in the below tables.

TABLE 33
Affinities of unilateral CD3/C-MET/EGFR trispecific
antibodies to EGFR (T = 30° C.)
Antigen: EGFR

| Sample | ka (1/Ms) | kdis (1/s) | KD (M) |
|---|---|---|---|
| EGFR-BsAb | 6.22E+05 | 4.01E−04 | 6.45E−10 |
| 801 | 4.02E+05 | 4.33E−04 | 1.08E−09 |
| 802 | 3.91E+05 | 3.16E−04 | 8.07E−10 |
| 803 | 5.27E+05 | 4.26E−04 | 8.10E−10 |
| 804 | 4.68E+05 | 3.77E−04 | 8.07E−10 |
| 805 | 5.15E+05 | 4.41E−04 | 8.57E−10 |
| 806 | 4.02E+05 | 3.93E−04 | 9.78E−10 |
| 807 | 3.89E+04 | 6.61E−04 | 1.70E−08 |
| 808 | 9.10E+04 | 7.12E−04 | 7.83E−09 |
| 809 | 4.54E+05 | 5.03E−04 | 1.11E−09 |
| 813 | 5.10E+04 | 6.89E−04 | 1.35E−08 |
| 814 | 1.11E+05 | 7.97E−04 | 7.19E−09 |
| 815 | 4.81E+05 | 5.66E−04 | 1.18E−09 |

TABLE 34
Affinities of unilateral CD3/C-MET/EGFR trispecific
antibodies to c-Met (T = 30° C.)
Antigen: c-Met

| Sample | ka (1/Ms) | kdis (1/s) | KD (M) |
|---|---|---|---|
| c-Met-BsAb | 7.02E+05 | 3.67E−04 | 5.23E−10 |
| 801 | 2.80E+05 | 3.88E−04 | 1.38E−09 |
| 802 | 2.25E+05 | 4.34E−04 | 1.93E−09 |
| 803 | 7.11E+05 | 3.78E−04 | 5.32E−10 |
| 804 | 5.47E+05 | 2.79E−04 | 5.10E−10 |
| 805 | 6.51E+05 | 4.00E−04 | 6.14E−10 |
| 806 | 4.96E+05 | 3.47E−04 | 7.00E−10 |
| 807 | 2.94E+05 | 1.38E−04 | 4.70E−10 |
| 808 | 8.31E+05 | 5.36E−04 | 6.45E−10 |
| 809 | 7.16E+05 | 4.47E−04 | 6.25E−10 |
| 813 | 2.65E+05 | 1.35E−04 | 5.09E−10 |
| 814 | 7.52E+05 | 4.91E−04 | 6.53E−10 |
| 815 | 3.69E+05 | 4.42E−04 | 1.20E−09 |

TABLE 35
Affinities of unilateral CD3/C-MET/EGFR trispecific
antibodies to CD3 (T = 30° C.)
Antigen: CD3ε/δ

| Sample | ka (1/Ms) | kdis (1/s) | KD (M) |
|---|---|---|---|
| EGFR-BsAb | 5.47E+05 | 3.02E−04 | 5.51E−09 |
| 801 | 2.28E+05 | 6.90E−04 | 3.03E−09 |
| 802 | 3.28E+05 | 1.03E−03 | 3.14E−09 |
| 803 | 3.71E+05 | 6.26E−04 | 1.69E−09 |
| 804 | 3.34E+05 | 2.37E−03 | 7.12E−09 |
| 805 | 3.08E+05 | 7.56E−04 | 2.45E−09 |
| 806 | 2.42E+05 | 1.86E−03 | 7.71E−09 |
| 807 | 2.13E+05 | 1.53E−03 | 7.20E−09 |
| 808 | 3.46E+05 | 8.13E−04 | 2.35E−09 |
| 809 | 2.66E+05 | 8.09E−04 | 3.04E−09 |
| 813 | 1.78E+05 | 8.83E−04 | 4.95E−09 |
| 814 | 3.05E+05 | 7.78E−04 | 2.55E−09 |
| 815 | 2.01E+05 | 6.98E−04 | 3.47E−09 |

### Example 19: Unilateral CD3/C-MET/EGFR Trispecific Antibodies Induce T Cell Mediated Killing of EGFR-Positive Tumor Target Cells

T cell mediated killing of target cells induced by trispecific antibodies was tested in human tumor cells NCI-H157(−21) (c-Met negative; EGFR negative), OSRC2 (c-Met low; EGFR high), UT44 (c-Met medium; EGFR high), UT33A (c-Met high; EGFR high), HT-29 (c-Met low; EGFR low), H-1975 (c-Met low; EGFR low). Human PBMCs were used as effectors and killing was detected after 72 h incubation with antibodies.

Briefly, tumor cells were digested with trypsin/EDTA, washed once with pre-chilled PBS, re-suspended in RPMI 1640 medium containing 10% FBS, and plated at a density of 5,000 cells/well in a flat-bottom 96-well plate (Corning 3599). After culturing for 4 h, 50 μL of serially diluted antibody solution was added to each well. Each concentration was added to three replicate wells. The antibodies were incubated with the tumor cells for more than 30 min to fully attach the proteins to the cells. PBMC cells (Reid Bio, 1521) were thawed quickly, resuscitated, added to 10 mL of RPMI 1640 medium containing 10% FBS, centrifuged at 1000 rpm for 5 min, and re-suspended after discarding the supernatant. Cell density was adjusted and 50 μL of PBMCs was added to each well of target cells to reach a final E:T ratio of 10:1. The 96-well plate was placed in a 37° C., 5% CO₂ incubator for 3 days.

After culturing for 3 days, the cell culture supernatant was carefully removed. The plate was pat dried with a paper towel. Serum-Free DMEM containing 10% CCK8 detection solution (Dojindo, CK04-20) was added to the 96-well plate and incubated at 37° C. for 2-4 hours. After 4 hours, the absorbance at 450 nm was detected using a microplate reader (BioTech, Synergy LX). The cell viability was calculated according to the below formula:

cell viability=[(As−Ab)/(Ac−Ab)]×100%.
As: experimental well (medium containing cells, CCK-8, and test antibodies);
Ac: control wells (medium containing cells, CCK-8, without test antibodies);
Ab: blank well (medium without cells and test antibodies, CCK-8).

Four-parameter fitting of cell viability was performed using GraphPad Prism software based on the below formula: Y=(A−D)/[1+(X/C)B]+D.
A: Estimation of the asymptote above the curve;
D: Estimation of the asymptote under the curve;
B: The slope of the curve;
C: The dose corresponding to half of the maximum binding (EC5);
Y is the detected cell viability;
X is the drug concentration.

The results showed that trispecific antibodies induced PBMCs to kill tumor antigen-positive cells. EC50 values related to cell killing ability calculated using GraphPad Prism are shown in the below table (“−” means negative, “+” means low expression, “++” means medium expression, “+++” means high expression). It can be seen that the cell killing effects of the unilateral CD3/C-MET/EGFR trispecific antibody are significantly stronger than those of the EGFR-BsAb and c-Met-BsAb bispecific antibodies.

TABLE 36
Killing effect of CD3/C-MET/EGFR trispecific antibodies on EGFR positive tumor cells (EC50, nM)

| | H157(−21) | OSRC2 | UT44 | UT33A | HT-29 | H-1975 |
|---|---|---|---|---|---|---|
| Sample | (EGFR−c-Met−) | (EGFR+++c-Met+) | (EGFR+++c-Met++) | (EGFR+++c-Met+++) | (EGFR+c-Met+) | (EGFR+c-Met+) |
| 801 | >100 | 3.26E−05 | 8.36E−05 | 9.62E−06 | 1.14E−05 | 1.01E−05 |
| 802 | >100 | 6.39E−04 | 1.01E−04 | 6.58E−05 | 6.28E−05 | 4.35E−05 |
| 803 | >100 | 1.49E−03 | 1.06E−04 | 3.74E−04 | 1.34E−05 | 4.20E−05 |
| 804 | >100 | 3.61E−04 | 3.02E−06 | 7.24E−05 | 9.16E−06 | 1.14E−05 |
| 805 | >100 | 2.47E−04 | 3.03E−06 | 8.18E−05 | 7.95E−06 | 8.75E−06 |
| 806 | >100 | 1.16E−04 | 2.46E−04 | 2.30E−05 | 3.35E−06 | 1.06E−05 |
| 807 | >100 | 5.85E−04 | 3.15E−05 | 3.03E−05 | 5.58E−06 | 1.01E−05 |
| 808 | >100 | 9.54E−03 | 3.01E−06 | 9.99E−05 | 2.59E−05 | 5.58E−06 |
| 809 | >100 | 2.16E−04 | 2.86E−05 | 2.18E−05 | 9.44E−06 | 1.45E−05 |
| 813 | >100 | 9.27E−05 | 1.60E−05 | 1.12E−05 | 9.15E−06 | 4.04E−06 |
| 814 | >100 | 2.06E−04 | 3.62E−05 | 1.30E−05 | 5.25E−06 | 8.99E−06 |
| 815 | >100 | 1.44E−02 | 7.56E−05 | 1.11E−04 | 3.84E−04 | 3.20E−06 |
| EGFR- | >100 | 2.45E−03 | 1.54E−04 | 3.98E−04 | 3.87E−04 | 3.20E−04 |
| BsAb | | | | | | |
| c- | >100 | 2.43E−01 | 1.56E−04 | 2.70E−03 | 5.18E−04 | 2.23E−04 |
| Met- | | | | | | |
| BsAb | | | | | | |

### Example 20: In Vivo Anti-Tumor Effects of CD3/C-MET/EGFR Trispecific Antibodies on HT-29 Human Colon Cancer Xenografts

Female NCG mice (NOD/ShiLtJGpt-Prkdc^{cm26Cd52}Il2rg^{cm26Cd22}/Gpt, GemPharmatech, Strain NO. T001475) (n=5) were subject to subcutaneous injection of 1×10⁶ HT-29 cells premixed with human PBMCs (E:T ratio 1:1, total volume 200 μl), to evaluate the treatment effect of the control bispecific antibodies and the trispecific antibodies. 1 h after subcutaneous co-transplantation of tumor cells/PBMC in mice, intravenous injection of 0.0325 mg/kg bispecific antibodies was administered twice a week, and the solvent control group (Vehicle) was given PBS buffer (administered 3 times in total). Tumor volumes and body weight data were measured weekly with digital calipers, and the data were recorded.

Relative Change of Body weight (RCB) was calculated based on the following formula: RCB (%)=[1−(Bi/B0)×100%] (Bi: the average weight of the mice on the i-day of administration, B0: average body weight of mice on day 0 of administration). Simultaneously tumor volumes was calculated using the following formula: long diameter×short diameter 2/2. Tumor growth inhibition rate (TGI_{TV}) was calculated using the following formula: TGI_{TV}(%)=[1−(Ti−T0)/(Vi−V0)]×100% (Ti: mean tumor volume of the treatment group on day i of administration; T0: mean tumor volume of the treatment group that can be measured for the first time; Vi: mean tumor volume of the solvent control group on day i of administration; V0: the mean value of the tumor volume that can be measured for the first time after administration of the solvent control group). A two-way ANOVA test was performed and P<0.05 means statistically significant.

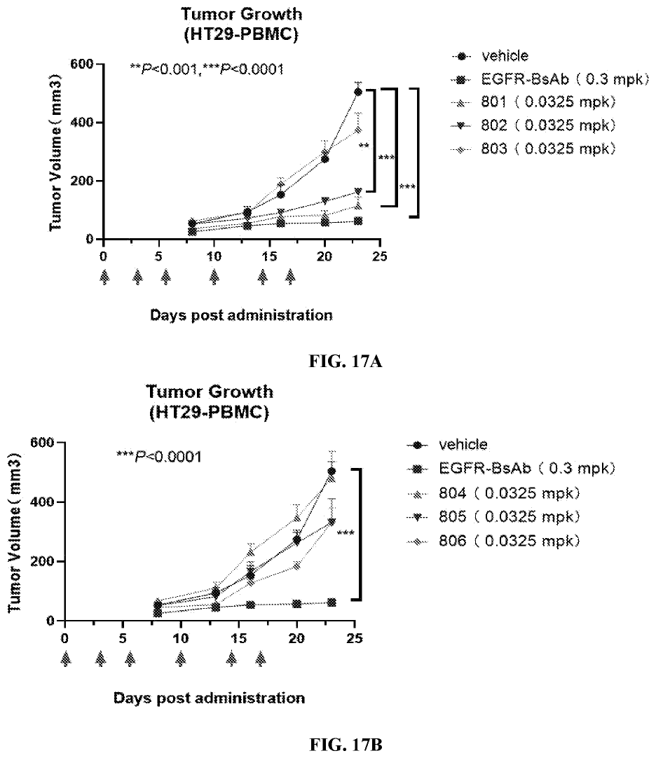

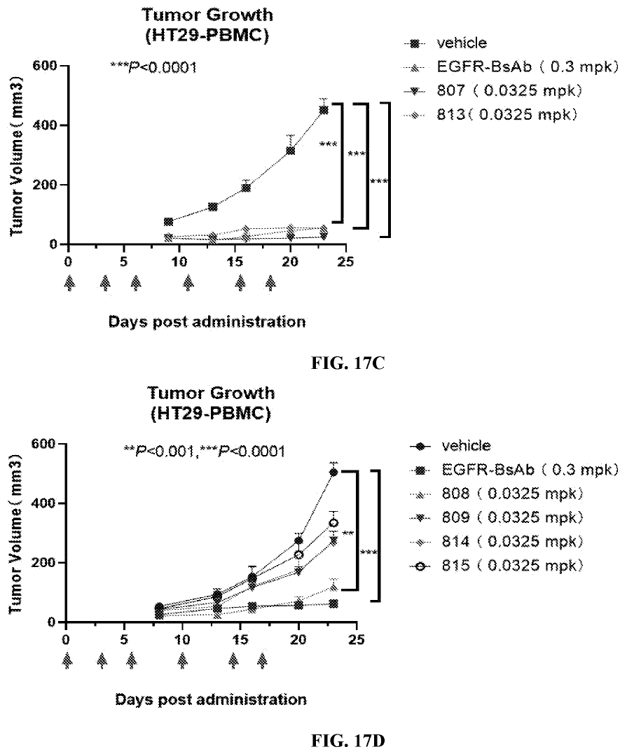

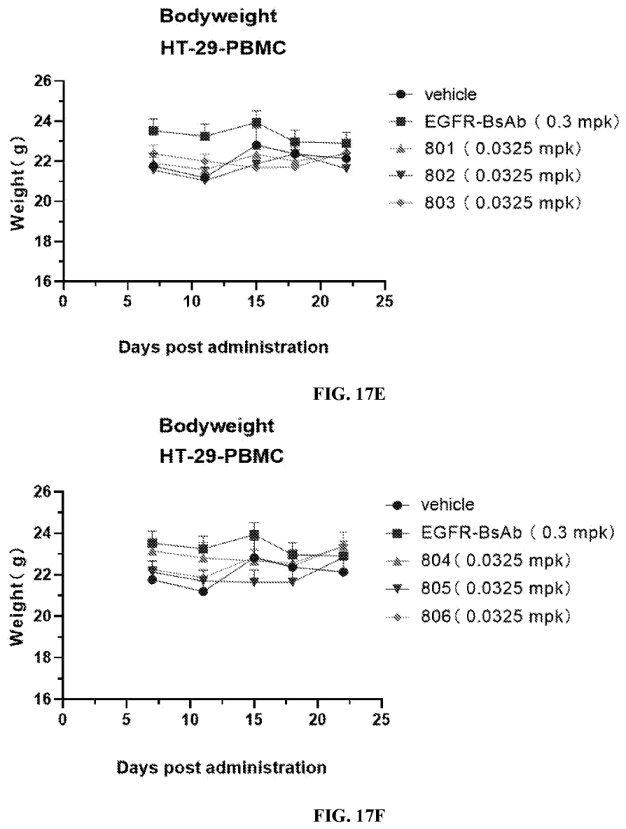

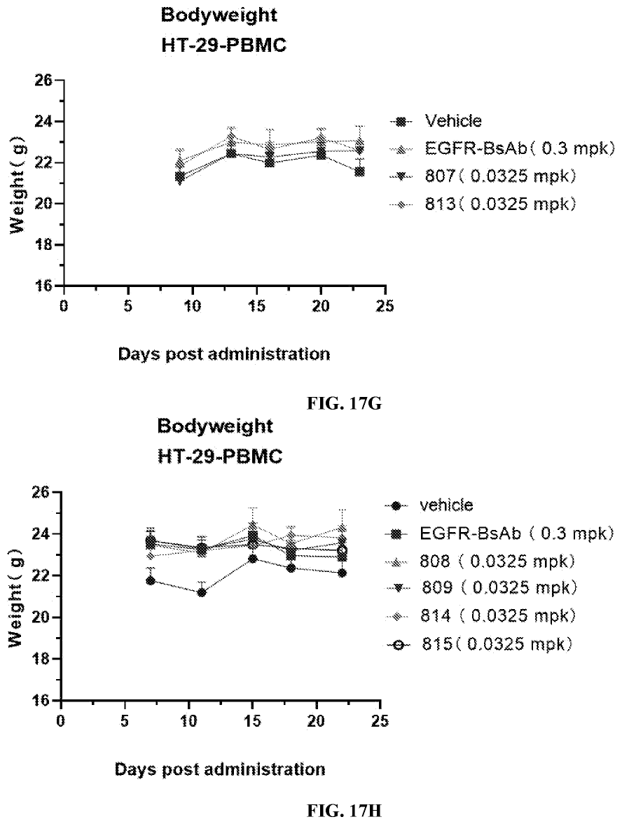

On the 23rd day of administration, compared with the vehicle control group, the CD3/C-MET/EGFR trispecific antibodies showed significant inhibitory effects on the tumor volume, with statistical significance (P<0.001). The results are shown in FIGS. 17A-17D and the below table. The tumor growth inhibition rates (TGI_{TV}) of 801, 802, 807, 808, and 813 at a dose of 0.0325 mg/kg were 85.33%, 78.11%, 96.33%, 76.91%, and 99.67%, respectively, indicating that the above five structures of the trispecific antibodies had strong tumor inhibitory effects. The TGI_{TV} of the control bispecific-antibody EGFR-BsAb at a dose of 0.3 mg/kg was 96.24%. The TGI_{TV} of the control bispecific-antibody EGFR-BsAb at a dose of 0.1 mg/kg was 16.54% (data not shown). TGI_{TV} of 7.5 mg/kg and 1.5 mg/kg c-Met-BsAb on the PBMC/HT-29 mixed inoculation mouse model was 60.30% and 43.39%, respectively, indicating that its tumor inhibitory effect was poor (data not shown).

TABLE 37
Effects of CD3/C-MET/EGFR trispecific antibodies on tumor
volume of NCG mice inoculated with PBMC/HT-29 mixture

| | Tumor Volume(mm³) | | | | | |
|---|---|---|---|---|---|---|
| Sample | Day 8 | Day 13 | Day 16 | Day 20 | Day 23 | TGITV(%) |
| Vehicle | 76.22 ± 6.21 | 126.09 ± 12.04 | 189.64 ± 26.66 | 314.52 ± 52.49 | 451.35 ± 38.16 | — |
| EGFR- | 22.71 ± 7.72 | 14.98 ± 7.33 | 26.19 ± 12.05 | 46.17 ± 11.13 | 54.21 ± 11.56 | 96.24% |
| BsAb(0.3 mpk) | | | | | | |
| 801(0.0325 | 36.02 ± 5.23 | 54.98 ± 17.23 | 78.75 ± 23.36 | 82.23 ± 15.57 | 115.18 ± 29.76 | 85.33% |
| mpk) | | | | | | |
| 802(0.0325 | 52.42 ± 8.22 | 72.52 ± 15.27 | 92.14 ± 19.52 | 130.24 ± 13.68 | 162.38 ± 11.7 | 78.11% |
| mpk) | | | | | | |
| 803(0.0325 | 63.22 ± 7.6 | 92.32 ± 10.29 | 189.32 ± 21.66 | 300.05 ± 38.05 | 373.25 ± 58.38 | 31.56% |
| mpk) | | | | | | |
| 804(0.0325 | 67.79 ± 9.57 | 110.84 ± 19.96 | 231.71 ± 28.54 | 347.71 ± 43.37 | 480.88 ± 90.21 | 9.86% |
| mpk) | | | | | | |
| 805(0.0325 | 51.7 ± 5.42 | 81.63 ± 12.91 | 166.42 ± 32.8 | 262.61 ± 42.79 | 331.93 ± 79.35 | 39.02% |
| mpk) | | | | | | |
| 806(0.0325 | 45.69 ± 3.2 | 55.2 ± 5.9 | 127.71 ± 12.61 | 183.01 ± 16.85 | 332.83 ± 47.75 | 32.37% |
| mpk) | | | | | | |
| 807(0.0325 | 20.19 ± 3 | 6.34 ± 2.52 | 15.29 ± 6.82 | 17.52 ± 6.56 | 21.41 ± 8.07 | 99.67% |
| mpk) | | | | | | |
| 808(0.0325 | 22.36 ± 3.31 | 25.08 ± 4.08 | 44.23 ± 10.39 | 71.15 ± 14.75 | 119.87 ± 25.19 | 76.91% |
| mpk) | | | | | | |
| 809(0.0325 | 43.49 ± 2.74 | 66.94 ± 4.3 | 117.13 ± 17.48 | 168.47 ± 17.55 | 275.09 ± 32.39 | 49.29% |
| mpk) | | | | | | |
| 813(0.0325 | 26.74 ± 4.18 | 31.39 ± 5.36 | 52 ± 7.6 | 56.01 ± 3.95 | 54.51 ± 4.63 | 94.37% |
| mpk) | | | | | | |
| 814(0.0325 | 39.22 ± 6.9 | 53.65 ± 11.35 | 120.85 ± 27.05 | 176.28 ± 42.82 | 267.71 ± 36.92 | 47.85% |
| mpk) | | | | | | |
| 815(0.0325 | 45.85 ± 5.23 | 86.44 ± 15.99 | 147.74 ± 41.68 | 227.35 ± 39.63 | 334.85 ± 37.87 | 39.49% |
| mpk) | | | | | | |

The NCG mice were in a good state in terms of activity and food intake during the administration period, and the relative body weight change rate was within ±5%. On the 23rd day of administration, the mice in the administration group had no significant weight loss. The above results showed that the mice tolerated the administration at this frequency and dosage, and the drug safety was good. The specific results are shown in FIGS. 17E-17H and the table below.

TABLE 38
Effects of antibodies on body weights of NCG mice inoculated with PBMC/HT-29

| | | | | | | Changes in body |
|---|---|---|---|---|---|---|
| | | | | | | weight after 23 |
| | | | | | | days of |
| | Body Weight (g) | | | | | administration |
| Sample | Day 8 | Day 13 | Day 16 | Day 20 | Day 23 | (g) |
| Vehicle | 21.35 ± 0.44 | 22.45 ± 0.57 | 21.98 ± 0.54 | 22.37 ± 0.61 | 21.57 ± 0.62 | +0.22 |
| EGFR-BsAb(0.3 mpk) | 22.1 ± 0.57 | 23 ± 0.74 | 22.87 ± 0.75 | 23.03 ± 0.65 | 23.07 ± 0.71 | +0.97 |
| 801(0.0325 mpk) | 21.92 ± 0.53 | 21.58 ± 0.47 | 22.35 ± 0.36 | 22.02 ± 0.54 | 22.37 ± 0.47 | +0.45 |
| 802(0.0325 mpk) | 21.57 ± 0.64 | 21.03 ± 0.69 | 21.88 ± 0.72 | 22.38 ± 0.7 | 21.63 ± 0.76 | +0.06 |
| 803(0.0325 mpk) | 22.38 ± 10.43 | 22.02 ± 0.34 | 21.68 ± 0.38 | 21.73 ± 0.96 | 22.43 ± 10.53 | +0.05 |
| 804(0.0325 mpk) | 23.15 ± 0.59 | 22.82 ± 0.75 | 22.67 ± 0.57 | 22.57 ± 0.66 | 23.38 ± 0.68 | +0.23 |
| 805(0.0325 mpk) | 22.13 ± 0.53 | 21.7 ± 0.53 | 21.63 ± 0.6 | 21.65 ± 0.75 | 22.85 ± 10.77 | +0.72 |
| 806(0.0325 mpk) | 22.25 ± 10.45 | 21.87 ± 0.34 | 22.85 ± 0.31 | 22.42 ± 0.34 | 23.4 ± 0.19 | +1.15 |
| 807(0.0325 mpk) | 21.12 ± 0.37 | 22.45 ± 0.49 | 22.28 ± 0.48 | 22.55 ± 0.54 | 22.58 ± 10.46 | +1.46 |
| 808(0.0325 mpk) | 23.47 ± 0.79 | 23.1 ± 0.75 | 24.45 ± 0.81 | 23.55 ± 0.82 | 24.32 ± 0.84 | +0.85 |
| 809(0.0325 mpk) | 23.68 ± 0.62 | 23.3 ± 0.6 | 23.75 ± 0.6 | 23.22 ± 0.49 | 23.58 ± 0.64 | −0.10 |
| 813(0.0325 mpk) | 21.87 ± 0.72 | 23.35 ± 0.36 | 22.92 ± 0.52 | 23.57 ± 0.52 | 22.95 ± 10.48 | +1.08 |
| 814(0.0325 mpk) | 22.93 ± 0.52 | 23.22 ± 0.48 | 23.45 ± 0.62 | 23.93 ± 0.37 | 23.82 ± 0.59 | +0.89 |
| 815(0.0325 mpk) | 23.68 ± 0.49 | 23.35 ± 0.37 | 23.5 ± 0.48 | 23.32 ± 0.3 | 23.22 ± 0.38 | −0.46 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antigen-binding protein, comprising
(a) a Fc;
(b) a Fab fragment (Fab) that specifically binds to a T cell antigen; and
(c) a first single-domain antibody variable domain (VHH) that specifically binds to a first tumor-associated antigen;
(d) a second single-domain antibody variable domain (VHH) that specifically binds to a second tumor-associated antigen,
wherein the Fab, the first VHH, and the second VHH are linked to the Fe.

2. The antigen-binding protein of claim 1, wherein the Fab comprises or consists of a light chain variable domain (VL), a light chain constant domain (CL), a heavy chain variable domain (VH), and a heavy chain first constant domain (CH1).

3. The antigen-binding protein of claim 2, wherein the Fab can activate T cells upon binding to the T cell antigen.

4. The antigen-binding protein of any one of claims 1-3, wherein the T cell antigen is cluster of differentiation 3 (CD3).

5. The antigen-binding protein of any one of claims 1-4, wherein the first tumor-associated antigen and the second tumor-associated antigen are independently selected from the group consisting of cluster of differentiate 20 (CD20), carcinoembryonic antigen (CEA), prostate-specific antigen (PSA), prostate stem cell antigen (PSCA), programmed death-ligand 1 (PD-L1), human epidermal growth factor receptor 2 (HER2), human epidermal growth factor receptor 3 (Her3), human epidermal growth factor receptor (Her1), β-Catenin, cluster of differentiate 19 (CD19), epidermal growth factor receptor (EGFR), tyrosine-protein kinase Met (c-Met), epithelial cell adhesion molecule (EPCAM), prostate-specific membrane antigen (PSMA), cluster of differentiate 40 (CD40), Mucin 1, Cell Surface Associated (MUC1), insulin-like growth factor 1 receptor (IGF1R), and carcinoembryonic antigen cell adhesion molecule 6 (CEA-CAM6).

6. The antigen-binding protein of any one of claims 1-5, wherein the Fc is human IgG4 Fc.

7. The antigen-binding protein of any one of claims 2-6, wherein the CH1 domain of the Fab is linked to a CH2 domain in the Fc, optionally via a hinge region.

8. The antigen-binding protein of claim 7, wherein the hinge region is a human IgG4 hinge region optionally with S228P mutation according to EU numbering.

9. The antigen-binding protein of any one of claims 1-8, wherein the first VHH is linked to a CH2 domain in the Fe, optionally via a hinge region.

10. The antigen-binding protein any one of claims 1-8, wherein the first VHH is linked to a CH3 domain in the Fe, optionally via a linker sequence.

11. The antigen-binding protein of any one of claims 1-10, wherein the second VHH is linked to a CH3 domain in the Fc, optionally via a linker sequence.

12. The antigen-binding protein of any one of claims 1-10, wherein the second VHH is linked to the Fab.

13. The antigen-binding protein of claim 12, wherein the second VHH is linked to the C-terminus of a CL domain in the Fab.

14. The antigen-binding protein of claim 12, wherein the second VHH is linked to the N-terminus of a VH domain in the Fab.

15. The antigen-binding protein of claim 12, wherein the second VHH is linked to the N-terminus of a VL domain in the Fab.

16. A protein complex, comprising:
(a) a first polypeptide comprising in the direction of N-terminus to C-terminus: a VH, a CH1 domain, optionally a first hinge region, a first CH2 domain, and a first CH3 domain;
(b) a second polypeptide comprising in the direction of N-terminus to C-terminus: a first VHH, optionally a second hinge region, a second CH2 domain, and a second CH3 domain;
(c) a third polypeptide comprising in the direction of N-terminus to C-terminus: a VL, and a CL domain,
wherein the VH and the VL associate with each other, forming an antigen binding site of a Fab that specifically binds to a T cell antigen, wherein the first VHH specifically binds to a first tumor-associated antigen.

17. The protein complex of claim 16, wherein the third polypeptide comprises in the direction of N-terminus to C-terminus: a VL, a CL domain, and a second VHH, wherein the second VHH specifically binds to a second tumor-associated antigen.

18. The protein complex of claim 16, wherein the third polypeptide comprises in the direction of N-terminus to C-terminus: a second VHH, a VL and a CL domain, wherein the second VHH specifically binds to a second tumor-associated antigen.

19. The protein complex of claim 16, wherein the first polypeptide comprises in the direction of N-terminus to C-terminus: a second VHH, a VH, a CH1 domain, optionally a first hinge region, a first CH2 domain, and a first CH3 domain, wherein the second VHH specifically binds to a second tumor-associated antigen.

20. The protein complex of claim 16, wherein the second polypeptide comprises in the direction of N-terminus to C-terminus: a first VHH, a second VHH, optionally a second hinge region, a second CH2 domain, and a second CH3 domain, wherein the second VHH specifically binds to a second tumor-associated antigen.

21. The protein complex of claim 16, wherein the second polypeptide comprises in the direction of N-terminus to C-terminus: a first VHH, optionally a second hinge region, a second CH2 domain, a second CH3 domain, and a second VHH, wherein the second VHH specifically binds to a second tumor-associated antigen.

22. The protein complex of claim 16, wherein the first polypeptide comprises in the direction of N-terminus to C-terminus: a first VHH, a second VHH, optionally a second hinge region, a second CH2 domain, a second CH3 domain, and a second VHH, wherein the second VHH specifically binds to a second tumor-associated antigen.

23. The protein complex of any one of claims 16-22, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

24. The protein complex of any one of claims 16-23, wherein the T cell antigen is CD3, and the first tumor-associated antigen is selected from the group consisting of: CEA, CEA-CAM6, HER2 and EGFR.

25. The protein complex of any one of claims 16-24, wherein the T cell antigen is CD3, and the second tumor-associated antigen is selected from the group consisting of: CEA, CEA-CAM6, HER2 and EGFR

26. A protein complex, comprising:
(a) a first polypeptide comprising in the direction of N-terminus to C-terminus: a first VHH, optionally a first hinge region, a first CH2 domain, and a first CH3 domain;
(b) a second polypeptide comprising in the direction of N-terminus to C-terminus: a VH, a CH1 domain, optionally a second hinge region, a second CH2 domain, and a second CH3 domain;
(c) a third polypeptide comprising in the direction of N-terminus to C-terminus: a VL, and a CL domain,
wherein the VH and the VL associate with each other, forming an antigen binding site of a Fab that specifically binds to a T cell antigen, wherein the first VHH specifically binds to a first tumor-associated antigen.

27. The protein complex of claim 26, wherein the third polypeptide comprises in the direction of N-terminus to C-terminus: a VL, a CL domain, and a second VHH, wherein the second VHH specifically binds to a second tumor-associated antigen.

28. The protein complex of claim 26, wherein the third polypeptide comprises in the direction of N-terminus to C-terminus: a second VHH, a VL and a CL domain, wherein the second VHH specifically binds to a second tumor-associated antigen.

29. The protein complex of claim 26, wherein the second polypeptide comprises in the direction of N-terminus to C-terminus: a second VHH, a VH, a CH1 domain, optionally a first hinge region, a first CH2 domain, and a first CH3 domain, wherein the second VHH specifically binds to a second tumor-associated antigen.

30. The protein complex of claim 26, wherein the first polypeptide comprises in the direction of N-terminus to C-terminus: a first VHH, a second VHH, optionally a second hinge region, a second CH2 domain, and a second CH3 domain, wherein the second VHH specifically binds to a second tumor-associated antigen.

31. The protein complex of claim 26, wherein the second polypeptide comprises in the direction of N-terminus to C-terminus: a VH, a CH1 domain, optionally a first hinge region, a first CH2 domain, a first CH3 domain, and a second VHH, wherein the second VHH specifically binds to a second tumor-associated antigen.

32. The protein complex of claim 26, wherein the first polypeptide comprises in the direction of N-terminus to C-terminus: a first VHH, optionally a second hinge region, a second CH2 domain, a second CH3 domain, and a second VHH, wherein the second VHH specifically binds to a second tumor-associated antigen.

33. The protein complex of any one of claims 26-32, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

34. The protein complex of any one of claims 26-33, wherein the T cell antigen is CD3, and the first tumor-associated antigen is selected from the group consisting of: CEA, CEA-CAM6, HER2 and EGFR.

35. The protein complex of any one of claims 26-34, wherein the T cell antigen is CD3, and the second tumor-associated antigen is selected from the group consisting of: CEA, CEA-CAM6, HER2 and EGFR.

36. A protein complex, comprising:
(a) a first polypeptide comprising in the direction of N-terminus to C-terminus: a VH, a CH1 domain, optionally a first hinge region, a first CH2 domain, a first CH3 domain, and a first VHH;
(b) a second polypeptide comprising in the direction of N-terminus to C-terminus:
optionally a second hinge region, a second CH2 domain, a second CH3 domain, and a second VHH; and
(c) a third polypeptide comprising in the direction of N-terminus to C-terminus: a VL, and a CL domain,
wherein the VH and the VL associate with each other, forming an antigen binding site of a Fab that specifically binds to a T cell antigen, wherein the first VHH specifically binds to a first tumor-associated antigen and the second VHH specifically binds to a second tumor-associated antigen.

37. The protein complex of claim 36, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

38. The protein complex of any one of claims 36-37, wherein the T cell antigen is CD3, and the first tumor-associated antigen is selected from the group consisting of: CEA, CEA-CAM6, HER2 and EGFR.

39. The protein complex of any one of claims 36-38, wherein the T cell antigen is CD3, and the second tumor-associated antigen is selected from the group consisting of: CEA, CEA-CAM6, HER2 and EGFR.

40. A protein complex, comprising:
(a) a first polypeptide comprising in the direction of N-terminus to C-terminus: optionally a first hinge region, a first CH2 domain, and a first CH3 domain, and a first VHH;
(b) a second polypeptide comprising in the direction of N-terminus to C-terminus: a VH, a CH1 domain, optionally a second hinge region, a second CH2 domain, a second CH3 domain, and a second VHH; and
(c) a third polypeptide comprising in the direction of N-terminus to C-terminus: a VL, and a CL domain,
wherein the VH and the VL associate with each other, forming an antigen binding site of a Fab that specifically binds to a T cell antigen, wherein the first VHH specifically binds to a first tumor-associated antigen and the second VHH specifically binds to a second tumor-associated antigen.

41. The protein complex of claim 40, wherein the first polypeptide comprises a sequence that is at least 80%, 90%, 95%, or 100% identical to SEQ ID NO: 1; the second polypeptide comprises a sequence that is at least 80%, 90%, 95%, or 100% identical to SEQ ID NO: 2; and the third polypeptide comprises a sequence that is at least 80%, 90%, 95%, or 100% identical to SEQ ID NO: 3.

42. The protein complex of any one of claims 40-41, wherein the T cell antigen is CD3, and the first tumor-associated antigen is selected from the group consisting of: CEA, CEA-CAM6, HER2 and EGFR.

43. The protein complex of any one of claims 40-42, wherein the T cell antigen is CD3, and the second tumor-associated antigen is selected from the group consisting of: CEA, CEA-CAM6, HER2 and EGFR.

44. The protein complex of any one of claims 16-43, wherein the first CH3 domain comprises one or more knob mutations, and the second CH3 domain comprises one or more hole mutations.

45. The protein complex of any one of claims 16-44, wherein the VH comprises a sequence that is at least 80%, 90%, 95%, or 100% identical to SEQ ID NO: 5 and the VL comprises a sequence that is at least 80%, 90%, 95%, or 100% identical to SEQ ID NO: 6.

46. The protein complex of any one of claims 16-45, wherein the VHH comprises a sequence that is at least 80%, 90%, 95%, or 100% identical to any one of SEQ ID NOs: 7-10.

47. The protein complex of any one of claims 16-46, wherein the VHH is linked to the CH3 domain via a linker peptide.

48. A nucleic acid comprising a polynucleotide encoding the antigen-binding protein of any one of claims 1-15, or the protein complex of any one of claims 16-47.

49. The nucleic acid of claim 48, wherein the nucleic acid is a DNA (e.g., cDNA) or RNA (e.g., mRNA).

50. A vector comprising one or more of the nucleic acids of claim 48 or 49.

51. A cell comprising the vector of claim 50.

52. The cell of claim 51, wherein the cell is a HEK293F cell or CHO cell.

53. A cell comprising one or more of the nucleic acids of claim 48 or 49.

54. A method of producing an antigen-binding protein or protein complex, the method comprising
(a) culturing the cell of any one of claims 51-53 under conditions sufficient for the cell to produce the antigen-binding protein or protein complex; and
(b) collecting the antigen-binding protein or protein complex produced by the cell.

55. An antibody-drug conjugate comprising the antigen-binding protein of any one of claims 1-15, or the protein complex of any one of claims 16-47, covalently bound to a therapeutic agent.

56. The antibody drug conjugate of claim 55, wherein the therapeutic agent is a cytotoxic or cytostatic agent.

57. A method of treating a subject having cancer, the method comprising administering a therapeutically effective amount of a composition comprising the antigen-binding protein of any one of claims 1-15, the protein complex of any one of claims 16-47, or the antibody-drug conjugate of claim 55 or 56, to the subject.

58. The method of claim 57, wherein the subject has a cancer expressing CEA-CAM6.

59. The method of claim 57 or 58, wherein the cancer is lung cancer, colorectal cancer, head and neck cancer, stomach cancer, pancreatic cancer, urothelial cancer, breast cancer, cervical cancer, or endometrial cancer.

60. A method of decreasing the rate of tumor growth, the method comprising contacting a tumor cell with an effective amount of a composition comprising the antigen-binding protein of any one of claims 1-15, the protein complex of any one of claims 16-47, or the antibody-drug conjugate of claim 55 or 56.

61. A method of killing a tumor cell, the method comprising contacting a tumor cell with an effective amount of a composition comprising the antigen-binding protein of any one of claims 1-15, the protein complex of any one of claims 16-47, or the antibody-drug conjugate of claim 55 or 56.

62. A pharmaceutical composition comprising the antigen-binding protein of any one of claims 1-15, or the protein complex of any one of claims 16-47, and a pharmaceutically acceptable carrier.


SEQUENCE LISTING (13 sequences)

SEQ ID NO: 1 - protein, 227 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 2 - protein, 227 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 3 - protein, 107 aa, synthetic construct [sequence omitted]

SEQ ID NO: 4 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 5 - protein, 125 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 6 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIK

SEQ ID NO: 7 - protein, 120 aa, synthetic construct
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 8 - protein, 122 aa, synthetic construct
QVQLQESGGGSVQAGGSLKLSCAASGRGFSRYAMGWFRQAPGQDREFVATISWTNSTDYADSVKGRFAISRDNAKNTAYLQMNSLKPEDTAVYYCAADKWASSTRSIDYDYWGQGIQVTVSS

SEQ ID NO: 9 - protein, 123 aa, synthetic construct
QVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS

SEQ ID NO: 10 - protein, 123 aa, synthetic construct [sequence omitted]

SEQ ID NO: 11 - protein, 10 aa, synthetic construct
GGGGSGGGGS

SEQ ID NO: 12 - protein, 12 aa, synthetic construct
ESKYGPPCPPCP

SEQ ID NO: 13 - protein, 122 aa, synthetic construct
QVKLEESGGDLVQPGGSLRLSCVGSGSIFSYTAMGWYRQAPGKQRELVATIHSGGNINYADSVKGRFTISRDNTENTVYLQMNSLKPEDTAVYYCWSLLVGPETRYKDFNFWGRGTQVTVSS